(19) United States
(12) Patent Application Publication
(10) Pub. No.: US 2004/0029133 A1
(43) Pub. Date: Feb. 12, 2004

(54) Mitochondrial DNA polymorphisms

(75) Inventor: Corinna Herrnstadt, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: MitoKor, Inc., San Diego, CA (US)

(21) Appl. No.: 10/308,264

(22) Filed: Nov. 25, 2002

Related U.S. Application Data
(60) Provisional application No. 60/369,539, filed on Apr. 1, 2002.
(60) Provisional application No. 60/369,131, filed on Mar. 28, 2002.
(60) Provisional application No. 60/333,622, filed on Nov. 26, 2001.

(51) Int. Cl. C12Q 1/68; C12P 19/34

(52) U.S. Cl. 435/6; 435/91.2

(57) ABSTRACT

Compositions and methods based on determination of single nucleotide polymorphisms in mtDNA or homoplasmic mtDNA mutations are provided that are useful for detecting the presence or risk of diseases, determining the haplogroup of an individual, and establishing genetic relationships between individuals for genealogical and forensic purposes.

### CROSS-REFERENCE TO RELATED APPLICATIONS

This application claims the benefit of U.S. Provisional Patent Application No. 60/369,539 filed Apr. 1, 2002; No. 60/369,131 filed Mar. 28, 2002; and No. 60/333,622 filed Nov. 26, 2001, which are incorporated herein by reference in their entirety.

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 461.app.txt which is 11.3 MB and created on Nov. 25, 2002; CD-ROM No.2 is labeled COPY 2, contains the file 461.app.txt which is 11.3 MB and created on Nov. 25, 2002; CD-ROM No. 3 is labeled CRF, contains the file 461.app.txt which is 11.3 MB and created on Nov. 25, 2002.

### TECHNICAL FIELD

The present invention relates generally to mitochondrial DNA polymorphisms and, more specifically, to compositions and methods based upon the identification of mitochondrial DNA polymorphisms for use in disease diagnosis, prognosis and treatment; patient and population profiling; pharmacogenomics; phylogenetic and population genetic analysis; genealogy; forensics and paternity testing; and related areas.

### BACKGROUND OF THE INVENTION

Mitochondria are the subcellular organelles that manufacture bioenergetically essential adenosine triphosphate (ATP) by oxidative phosphorylation. Functional mitochondria contain gene products encoded by mitochondrial genes situated in mitochondrial DNA (mtDNA) and by extramitochondrial genes not situated in the circular mitochondrial genome. The 16.5 kb mtDNA encodes 22 tRNAs, two ribosomal RNAs (12s and 16s rRNA) and only 13 enzymes of the electron transport chain (ETC), the elaborate multi-subunit complex mitochondrial assembly where, for example, respiratory oxidative phosphorylation takes place. (See, e.g., Wallace et al., in Mitochondria & Free Radicals in Neurodegenerative Diseases, M. F. Beal, N. Howell and I. Bodis-Wollner, eds., 1997 Wiley-Liss, Inc., New York, pp. 283-307, and references cited therein; see also, e.g., Scheffler, I. E., Mitochondria, 1999 Wiley-Liss, Inc., New York.) More than 5000 copies of the mitochondrial genome may be present within a single cell, due to the presence of numerous mitochondria within a single cell, and of multiple copies of mtDNA within each mitochondrion. Furthermore, since mitochondrial DNA is strictly maternally inherited, all copies of mitochondrial DNA within an individual are generally monoclonal. Finally, certain regions of mtDNA are highly polymorphic. Mitochondrial DNA includes gene sequences encoding a number of ETC components, including seven subunits of NADH dehydrogenase, also known as ETC Complex I (ND1, ND2, ND3, ND4, ND4L, ND5 and ND6); one subunit of Complex III (ubiquinol: cytochrome c oxidoreductase, Cytb); three cytochrome c oxidase (Complex IV) subunits (COX1, COX2 and COX3); and two proton-translocating ATP synthase (Complex V) subunits (ATPase6 and ATPase8).

The first complete sequence of a human mitochondrial DNA (mtDNA), also referred to as the Cambridge reference sequence (CRS), was originally published in 1981 and was more recently revised (Anderson, S. et al., Nature 290:457-465, 1981; Andrews, R. M. et al., Nature Genetics 23:147, 1999). The mtDNA is strictly maternally inherited and has a mutation rate ten times that of nuclear DNA, with certain regions exhibiting notably high degrees of polymorphism. During human evolution and subsequent colonization of the continents by human subpopulations, the genealogic pattern of mtDNA inheritance indicates divergence of distinct maternal lineages, which are observed to harbor population-specific mtDNA polymorphisms. Thus, nine different European mtDNA haplogroups (e.g., discrete constellations of homoplasmic mtDNA polymorphisms that are highly conserved among members of a common maternal lineage as well as distinct Asian, Native American and African mtDNA haplogroups, have been described on the basis of the presence or absence in mtDNA of one or several restriction endonuclease recognition sites (described in Wallace et al., Gene 238:211-230, 1999; Torroni, A. et al., Genetics 144:1835-1850, 1996).

A number of degenerative diseases are thought to be caused by, or are associated with, alterations in mitochondrial function. These diseases include Alzheimer's Disease, diabetes mellitus, Parkinson's Disease, Huntington's disease, dystonia, Leber's hereditary optic neuropathy, schizophrenia, and myodegenerative disorders such as “mitochondrial encephalopathy, lactic acidosis, and stroke” (MELAS), and “myoclonic epilepsy ragged red fiber syndrome” (MERRF). Other diseases involving altered metabolism or respiration within cells may also be regarded as diseases associated with altered mitochondrial function. The extensive list of additional diseases associated with altered mitochondrial function continues to expand as aberrant mitochondrial or mitonuclear activities are implicated in particular disease processes.

There is evidence to suggest that the genetic basis of at least some diseases associated with altered mitochondrial function resides in mitochondrial DNA rather than in extramitochondrial DNA such as that found in the nucleus (e.g., nuclear chromosomal and extrachromosomal DNA). For example, noninsulin dependent diabetes mellitus (NIDDM) exhibits a predominantly maternal pattern of inheritance, and in at least some cases this disease appears to be associated with a mitochondrial DNA (mtDNA) abnormality not found in the CRS or the revised CRS. Thus, for instance, approximately 1.5% of all diabetic individuals harbor a mutation at mtDNA position 3243 in the mitochondrial gene encoding leucyl-tRNA (tRNA^{Leu}). This mutation is known as the MELAS (mitochondrial encephalopathy, lactic acidosis and stroke) mutation. (Gerbitz et al., Biochim. Biophys. Acta 1271:253-260, 1995.) Similar theories have been advanced for analogous relationships between mtDNA mutations and other diseases associated with altered mitochondrial function, including but not limited to Alzheimer's Disease (AD), Huntington's Disease (HD), Parkinson's Disease (PD), dystonia, Leber's hereditary optic neuropathy (LHON), schizophrenia, and myoclonic epilepsy ragged red fiber syndrome (MERRF) (See, e.g., Chinnery et al., 1999 J. Med. Genet. 36:425). In addition, a limited number of specific single nucleotide polymorphisms that correlate with Alzheimer's disease or with type 2 diabetes mellitus have been identified (see U.S. patent application Ser. No. 09/551,941; see also co-pending application serial No. 60/333,448). Certain somatic mtDNA sequence changes have also been detected in tissues from colorectal cancer (Polyack et al., (1998) Nature Genetics 20:291-293) and in lung, bladder, and head and neck tumors (Fliss et al., (1999) Science 287:2017-2019). The identification of additional mtDNA mutations associated with diseases may provide targets for the development of diagnostic and/or therapeutic agents.

As is well known in the art generally, and especially with regard to extramitochondrial DNA such as nuclear chromosomal and/or extrachromosomal DNA, direct (e.g., by nucleotide sequencers) or indirect (e.g., by RFLP, SSCP, etc.) determination of DNA sequence variations in individuals and in populations has been used for a wide range of purposes. For example, identification of variability at specific marker loci is useful in a wide range of genetic studies (e.g., genetic counseling, diagnosis of inherited disorders and/or of cancer, pharmacogenetics, etc.), in commercial breeding, in genotyping of samples (e.g., for transplantation, transfusion, cell or tissue grafting, etc.), forensic analysis, paternity testing and the like.

Currently available DNA-based identification technology makes use of a variety of DNA fingerprinting techniques (for a discussion of common procedures, see Murch, R. S. and Budowle, B., (1997) Are Developments in Forensic Applications ofDNA Technology Consistent with Privacy Protection? in Genetic Secrets: Protecting Privacy and Confidentiality in the Genetic Era (ed. Mark A. Rothstein), Yale University Press, New Haven, Conn.). DNA fingerprinting includes a variety of methods for assessing sequence differences in DNA isolated from various sources, e.g., by comparing the presence of marker DNA in samples of isolated DNA. Typically, DNA fingerprinting is used to analyze and compare DNA from different species of organisms, or from different individuals of the same species. Many technologies have been used in DNA fingerprinting, including, inter alia, restriction fragment length polymorphism (RFLP; e.g., Bostein et al. (1980) Am. J. Hum. Genet. 32:314-331), single strand conformation polymorphism (SSCP; Fischer et al. (1983) Proc. Natl. Acad. Sci. USA 80:1579-1583; Orita et al. (1989) Genomics 5:874-879), amplified fragment-length polymorphism (AFLP; Vos et al. (1995) Nucleic Acids Res. 23:4407-4414), microsatellite or single-sequence repeat analysis (SSR; Weber J L and May P E (1989) Am. J. Hum. Genet. 44:388-396), rapid-amplified polymorphic DNA analysis (RAPD; Williams et al. (1990) Nucleic Acids Res. 18:6531-6535), sequence tagged site analysis (STS; Olson et al. (1989) Science 245:1434-1435), genetic-bit analysis (GBA; Nikiforov et al. (1994) Nucleic Acids Res 22:4167-4175), allele-specific polymerase chain reaction (ASPCR; Gibbs et al. (1989) Nucleic Acids Res. 17:2437-2448; Newton et al. (1989) Nucleic Acids Res. 17:2503-2516), nick-translation PCR (e.g., TaqMan™; Lee et al. (1993) Nucleic Acids Res. 21:3761-3766), and allele-specific hybridization (ASH; Wallace et al. (1979) Nucleic Acids Res. 6:3543-3557; Sheldon et al. (1993) Clinical Chemistry 39(4):718-719) among others.

According to certain commonly used methods of DNA fingerprinting, variable number tandem repeat (VNTR) regions within genomic DNA are genetically characterized by restriction fragment length polymorphisms (RFLP) analysis. Alternative approaches, for example, those that are generally based upon use of the polymerase chain reaction (PCR), include dot-blot assays, electrophoretic analysis and direct nucleic acid sequencing. By such methods, a variety of genomic DNA sequence polymorphisms can be analyzed, including, for example, polymorphisms in HLA-DQA1 (or other loci of the highly polymorphic major histocompatibility complex (MHC)), low-density lipoprotein receptor (LDL-R), glycophorin A, hemoglobin G gammaglobin, D7S8 and other group-specific components.

When such techniques are directed to analysis of nuclear (e.g., chromosomal) DNA, however, certain limitations become apparent, including, for instance, (1) the availability of only a limited amount of sample material because there are only two copies of each gene per cell, and (2) the presence in a sample of two different nucleotide sequences at a particular genetic locus where the subject is heterozygous at that locus (e.g., by having inherited different allelic forms of the gene from each parent). Given the devastating consequences of human diseases such as, for example, Alzheimer's disease and type 2 diabetes mellitus, clearly there is a need to develop improved compositions and methods for identifying the presence of, or risk for having, such diseases in individuals. In addition, there is a clear need in the art to develop more sensitive and reliable compositions and methods for use in forensics and in other applications requiring the identification of individuals and/or their genetic relationships to others. The present invention addresses these needs by providing compositions and methods that employ mtDNA as a source of genetic markers for diagnostic, prognostic, pharmacogenetic, evolutionary, forensic and/or genealogic analyses, and offers other related advantages.

### SUMMARY OF THE INVENTION

The present invention is directed in part to compositions and methods that relate to identification of mitochondrial DNA polymorphisms. Accordingly, it is an aspect of the invention to provide a method for determining the mitochondrial haplogroup of a subject, comprising determining, in a biological sample comprising mitochondrial DNA from a subject, the presence or absence of at least one mitochondrial single nucleotide polymorphism that is associated with a mitochondrial haplogroup. In certain embodiments the mitochondrial DNA is amplified. In certain other embodiments, at least one mitochondrial single nucleotide polymorphism that is associated with a mitochondrial haplogroup is present in a mitochondrial DNA region that is a D-loop, a mitochondrial rRNA gene, a mitochondrial NADH dehydrogenase gene, a mitochondrial tRNA gene, a mitochondrial cytochrome c oxidase gene, a mitochondrial ATP synthase gene or a mitochondrial cytochrome b gene.

In certain other embodiments at least one mitochondrial single nucleotide polymorphism that is associated with a mitochondrial haplogroup is a haplogroup-specific polymorphism or a haplogroup-associated polymorphism, wherein a mitochondrial single nucleotide polymorphism that is haplogroup-specific is, for A, B, C, D, E, H, I, J, K, L1, L2, L3, T, U, V, W or X, located at a nucleotide that corresponds to a nucleotide position of SEQ ID NO:1 that is selected from:

| Nucleotide | | |
|---|---|---|
| Position | GENE | HAPLOGROUP |
| 64 | D-LOOP | A |
| 235 | D-LOOP | A |
| 663 | 12S rRNA | A |
| 1598 | 12S rRNA | A |
| 1736 | 16S rRNA | A |
| 3316 | ND1 | A |
| 4248 | ND1 | A |
| 4824 | ND2 | A |
| 4970 | ND2 | A |
| 6308 | COI | A |
| 7112 | COI | A |
| 7724 | COII | A |
| 8794 | ATPase6 | A |
| 11314 | ND4 | A |
| 12468 | ND5 | A |
| 12811 | ND5 | A |
| 13855 | ND5 | A |
| 14364 | ND6 | A |
| 16111 | D-LOOP | A |
| 16290 | D-LOOP | A |
| 16319 | D-LOOP | A |
| 827 | 12S rRNA | B |
| 3547 | ND1 | B |
| 4820 | ND2 | B |
| 4977 | ND2 | B |
| 6023 | COI | B |
| 6216 | COI | B |
| 6413 | COI | B |
| 6473 | COI | B |
| 6755 | COI | B |
| 7241 | COI | B |
| 9950 | COIII | B |
| 11177 | ND4 | B |
| 15535 | CYT B | B |
| 16217 | D-LOOP | B |
| 249 | D-LOOP | C |
| 289 | D-LOOP | C |
| 290 | D-LOOP | C |
| 3552 | ND1 | C |
| 4715 | ND2 | C |
| 7196 | COI | C |
| 7694 | COII | C |
| 8584 | ATPase6 | C |
| 9545 | COII | C |
| 10454 | tRNA-R | C |
| 12642 | ND5 | C |
| 12978 | ND5 | C |
| 14318 | ND6 | C |
| 15487 | CYT B | C |
| 15930 | tRNA-T | C |
| 2092 | 16S rRNA | D |
| 4883 | ND2 | D |
| 5178 | ND2 | D |
| 8414 | ATPase8 | D |
| 11593 | ND4 | D |
| 14668 | ND6 | D |
| 3027 | 16S rRNA | E |
| 3705 | ND1 | E |
| 4491 | ND2 | E |
| 7598 | COII | E |
| 239 | D-LOOP | H |
| 456 | D-LOOP | H |
| 477 | D-LOOP | H |
| 951 | 12S rRNA | H |
| 961 | 12S rRNA | H |
| 3277 | tRNA-L | H |
| 3333 | ND1 | H |
| 3591 | ND1 | H |
| 3796 | ND1 | H |
| 3915 | ND1 | H |
| 3992 | ND1 | H |
| 4024 | ND1 | H |
| 4310 | tRNA-I | H |
| 4336 | tRNA-Q | H |
| 4531 | ND2 | H |
| 4727 | ND2 | H |
| 4745 | ND2 | H |
| 4772 | ND2 | H |
| 4793 | ND2 | H |
| 5004 | ND2 | H |
| 6365 | COI | H |
| 6776 | COI | H |
| 6869 | COI | H |
| 7013 | COI | H |
| 8269 | COII | H |
| 8448 | ATPase8 | H |
| 8602 | ATPase6 | H |
| 8803 | ATPase6 | H |
| 8839 | ATPase6 | H |
| 8843 | ATPase6 | H |
| 8898 | ATPase6 | H |
| 9123 | ATPase6 | H |
| 9150 | ATPase6 | H |
| 9380 | COIII | H |
| 9804 | COIII | H |
| 10044 | tRNA-G | H |
| 11353 | ND4 | H |
| 11560 | ND4 | H |
| 12579 | ND5 | H |
| 13404 | ND5 | H |
| 13680 | ND5 | H |
| 13759 | ND5 | H |
| 14125 | ND5 | H |
| 14350 | ND6 | H |
| 14365 | ND6 | H |
| 14470 | ND6 | H |
| 14582 | ND6 | H |
| 14872 | CYT B | H |
| 15466 | CYT B | H |
| 15789 | CYT B | H |
| 15808 | CYT B | H |
| 15833 | CYT B | H |
| 16162 | D-LOOP | H |
| 16293 | D-LOOP | H |
| 199 | D-LOOP | I |
| 250 | D-LOOP | I |
| 3447 | ND1 | I |
| 3990 | ND1 | I |
| 4529 | ND2 | I |
| 6734 | COI | I |
| 8616 | ATPase6 | I |
| 9947 | COIII | I |
| 10034 | tRNA-G | I |
| 10238 | ND3 | I |
| 11065 | ND4 | I |
| 12501 | ND5 | I |
| 13780 | ND5 | I |
| 15758 | CYT B | I |
| 16391 | D-LOOP | I |
| 228 | D-LOOP | J |
| 295 | D-LOOP | J |
| 462 | D-LOOP | J |
| 2158 | 16S rRNA | J |
| 2387 | 16S rRNA | J |
| 3394 | ND1 | J |
| 5198 | ND2 | J |
| 5633 | tRNA-A | J |
| 6464 | COI | J |
| 6554 | COI | J |
| 6671 | COI | J |
| 7476 | tRNA-S | J |
| 7711 | COII | J |
| 10084 | ND3 | J |
| 10172 | ND3 | J |
| 10192 | ND3 | J |
| 10499 | ND4L | J |
| 10598 | ND4L | J |
| 10685 | ND4L | J |
| 11377 | ND4 | J |
| 12127 | ND4 | J |
| 12570 | ND5 | J |
| 12612 | ND5 | J |
| 13281 | ND5 | J |
| 13681 | ND5 | J |
| 13879 | ND5 | J |
| 13933 | ND5 | J |
| 14569 | ND6 | J |
| 15679 | CYT B | J |
| 15812 | CYT B | J |
| 16069 | D-LOOP | J |
| 16092 | D-LOOP | J |
| 16261 | D-LOOP | J |
| 114 | D-LOOP | K |
| 497 | D-LOOP | K |
| 593 | tRNA-F | K |
| 1189 | 12S rRNA | K |
| 2217 | 16S rRNA | K |
| 2483 | 16S rRNA | K |
| 3480 | ND1 | K |
| 4295 | tRNA-I | K |
| 4561 | ND2 | K |
| 5814 | tRNA-C | K |
| 6260 | COI | K |
| 9006 | ATPase6 | K |
| 9055 | ATPase6 | K |
| 9698 | COIII | K |
| 9716 | COIII | K |
| 9962 | COIII | K |
| 10289 | ND3 | K |
| 10550 | ND4L | K |
| 10978 | ND4 | K |
| 11299 | ND4 | K |
| 11470 | ND4 | K |
| 11485 | ND4 | K |
| 11840 | ND4 | K |
| 11869 | ND4 | K |
| 11923 | ND4 | K |
| 12954 | ND5 | K |
| 13135 | ND5 | K |
| 13740 | ND5 | K |
| 13967 | ND5 | K |
| 14002 | ND5 | K |
| 14037 | ND5 | K |
| 14040 | ND5 | K |
| 14167 | ND6 | K |
| 15884 | CYT B | K |
| 15946 | tRNA-T | K |
| 16224 | D-LOOP | K |
| 16234 | D-LOOP | K |
| 16463 | D-LOOP | K |
| 185 | D-LOOP | L1 |
| 186 | D-LOOP | L1 |
| 189 | D-LOOP | L1 |
| 236 | D-LOOP | L1 |
| 247 | D-LOOP | L1 |
| 297 | D-LOOP | L1 |
| 357 | D-LOOP | L1 |
| 710 | 12S rRNA | L1 |
| 825 | 12S rRNA | L1 |
| 1048 | 12S rRNA | L1 |
| 1738 | 16S rRNA | L1 |
| 2245 | 16S rRNA | L1 |
| 2395 | 16S rRNA | L1 |
| 2758 | 16S rRNA | L1 |
| 2768 | 16S rRNA | L1 |
| 2885 | 16S rRNA | L1 |
| 3308 | ND1 | L1 |
| 3516 | ND1 | L1 |
| 3666 | ND1 | L1 |
| 3693 | ND1 | L1 |
| 3777 | ND1 | L1 |
| 3796 | ND1 | L1 |
| 3843 | ND1 | L1 |
| 4312 | tRNA-I | L1 |
| 4586 | ND2 | L1 |
| 5036 | ND2 | L1 |
| 5393 | ND2 | L1 |
| 5442 | ND2 | L1 |
| 5603 | tRNA-A | L1 |
| 5655 | tRNA-A | L1 |
| 5913 | COI | L1 |
| 5951 | COI | L1 |
| 6071 | COI | L1 |
| 6150 | COI | L1 |
| 6185 | COI | L1 |
| 6253 | COI | L1 |
| 6548 | COI | L1 |
| 6827 | COI | L1 |
| 6989 | COI | L1 |
| 7055 | COI | L1 |
| 7076 | COI | L1 |
| 7146 | COI | L1 |
| 7337 | COI | L1 |
| 7389 | COI | L1 |
| 7867 | COII | L1 |
| 8248 | COII | L1 |
| 8428 | ATPase8 | L1 |
| 8655 | ATPase6 | L1 |
| 8784 | ATPase6 | L1 |
| 8877 | ATPase6 | L1 |
| 9042 | ATPase6 | L1 |
| 9072 | ATPase6 | L1 |
| 9347 | COIII | L1 |
| 9755 | COIII | L1 |
| 9818 | COIII | L1 |
| 10321 | ND3 | L1 |
| 10586 | ND4L | L1 |
| 10589 | ND4L | L1 |
| 10664 | ND4L | L1 |
| 10688 | ND4L | L1 |
| 10792 | ND4 | L1 |
| 10793 | ND4 | L1 |
| 10810 | ND4 | L1 |
| 11176 | ND4 | L1 |
| 11641 | ND4 | L1 |
| 11654 | ND4 | L1 |
| 11899 | ND4 | L1 |
| 12007 | ND4 | L1 |
| 12049 | ND4 | L1 |
| 12519 | ND5 | L1 |
| 12720 | ND5 | L1 |
| 12810 | ND5 | L1 |
| 13149 | ND5 | L1 |
| 13276 | ND5 | L1 |
| 13485 | ND5 | L1 |
| 13506 | ND5 | L1 |
| 13789 | ND5 | L1 |
| 13880 | ND5 | L1 |
| 13980 | ND5 | L1 |
| 14000 | ND5 | L1 |
| 14148 | ND5 | L1 |
| 14178 | ND6 | L1 |
| 14203 | ND6 | L1 |
| 14308 | ND6 | L1 |
| 14560 | ND6 | L1 |
| 14769 | CYT B | L1 |
| 14911 | CYT B | L1 |
| 15115 | CYT B | L1 |
| 15136 | CYT B | L1 |
| 16148 | D-LOOP | L1 |
| 16187 | D-LOOP | L1 |
| 16188 | D-LOOP | L1 |
| 16230 | D-LOOP | L1 |
| 16264 | D-LOOP | L1 |
| 16265 | D-LOOP | L1 |
| 16360 | D-LOOP | L1 |
| 16527 | D-LOOP | L1 |
| 143 | D-LOOP | L2 |
| 1442 | 12S rRNA | L2 |
| 1706 | 16S rRNA | L2 |
| 2332 | 16S rRNA | L2 |
| 2358 | 16S rRNA | L2 |
| 2416 | 16S rRNA | L2 |
| 2789 | 16S rRNA | L2 |
| 3495 | ND1 | L2 |
| 3918 | ND1 | L2 |
| 4158 | ND1 | L2 |
| 4185 | ND1 | L2 |
| 4370 | tRNA-Q | L2 |
| 4767 | ND2 | L2 |
| 5027 | ND2 | L2 |
| 5285 | ND2 | L2 |
| 5331 | ND2 | L2 |
| 5581 | tRNA-W | L2 |
| 5744 | NON-CODING | L2 |
| 6713 | COI | L2 |
| 7175 | COI | L2 |
| 7274 | COI | L2 |
| 7624 | COII | L2 |
| 7771 | COII | L2 |
| 8080 | COII | L2 |
| 8206 | COII | L2 |
| 8387 | ATPase8 | L2 |
| 8541 | ATPase8 | L2 |
| 8790 | ATPase6 | L2 |
| 8925 | ATPase6 | L2 |
| 9221 | COIII | L2 |
| 10115 | ND3 | L2 |
| 11944 | ND4 | L2 |
| 12236 | tRNA-S | L2 |
| 12630 | ND5 | L2 |
| 12693 | ND5 | L2 |
| 12948 | ND5 | L2 |
| 13803 | ND5 | L2 |
| 14059 | ND5 | L2 |
| 14544 | ND6 | L2 |
| 14566 | ND6 | L2 |
| 14599 | ND6 | L2 |
| 15110 | CYT B | L2 |
| 15217 | CYT B | L2 |
| 15229 | CYT B | L2 |
| 15236 | CYT B | L2 |
| 15244 | CYT B | L2 |
| 15391 | CYT B | L2 |
| 15629 | CYT B | L2 |
| 15945 | tRNA-T | L2 |
| 16114 | D-LOOP | L2 |
| 16213 | D-LOOP | L2 |
| 16309 | D-LOOP | L2 |
| 16390 | D-LOOP | L2 |
| 200 | D-LOOP | L3 |
| 2000 | 16S rRNA | L3 |
| 3438 | ND1 | L3 |
| 3450 | ND1 | L3 |
| 5773 | tRNA-C | L3 |
| 6524 | COI | L3 |
| 6587 | COI | L3 |
| 6680 | COI | L3 |
| 7424 | COI | L3 |
| 7618 | COII | L3 |
| 8616 | ATPase6 | L3 |
| 8618 | ATPase6 | L3 |
| 8650 | ATPase6 | L3 |
| 9554 | COIII | L3 |
| 10086 | ND3 | L3 |
| 10373 | ND3 | L3 |
| 10667 | ND4L | L3 |
| 10819 | ND4 | L3 |
| 11800 | ND4 | L3 |
| 13101 | ND5 | L3 |
| 13886 | ND5 | L3 |
| 13914 | ND5 | L3 |
| 14152 | ND6 | L3 |
| 14212 | ND6 | L3 |
| 14284 | ND6 | L3 |
| 15099 | CYT B | L3 |
| 15311 | CYT B | L3 |
| 15670 | CYT B | L3 |
| 15824 | CYT B | L3 |
| 15942 | tRNA-T | L3 |
| 15944 | tRNA-T | L3 |
| 16124 | D-LOOP | L3 |
| 16327 | D-LOOP | L3 |
| 930 | 12S rRNA | T |
| 2141 | 16S rRNA | T |
| 2850 | 16S rRNA | T |
| 4688 | ND2 | T |
| 4917 | ND2 | T |
| 5277 | ND2 | T |
| 6489 | COI | T |
| 7022 | COI | T |
| 8572 | ATPase6 | T |
| 8697 | ATPase6 | T |
| 9117 | ATPase6 | T |
| 9899 | COIII | T |
| 10463 | tRNA-R | T |
| 11242 | ND4 | T |
| 11812 | ND4 | T |
| 12633 | ND5 | T |
| 13368 | ND5 | T |
| 13758 | ND5 | T |
| 13965 | ND5 | T |
| 14233 | ND6 | T |
| 14687 | tRNA-E | T |
| 14905 | CYT B | T |
| 15028 | CYT B | T |
| 15274 | CYT B | T |
| 15607 | CYT B | T |
| 15928 | tRNA-T | T |
| 16163 | D-LOOP | T |
| 16182 | D-LOOP | T |
| 16186 | D-LOOP | T |
| 16294 | D-LOOP | T |
| 16296 | D-LOOP | T |
| 16324 | D-LOOP | T |
| 988 | 12S rRNA | U |
| 1700 | 16S rRNA | U |
| 1721 | 16S rRNA | U |
| 2294 | 16S rRNA | U |
| 3116 | 16S rRNA | U |
| 3197 | 16S rRNA | U |
| 3348 | ND1 | U |
| 3720 | ND1 | U |
| 3849 | ND1 | U |
| 4553 | ND2 | U |
| 4646 | ND2 | U |
| 4703 | ND2 | U |
| 4732 | ND2 | U |
| 4811 | ND2 | U |
| 5319 | ND2 | U |
| 5390 | ND2 | U |
| 5495 | ND2 | U |
| 5656 | NON-CODING | U |
| 5999 | COI | U |
| 6045 | COI | U |
| 6047 | COI | U |
| 6146 | COI | U |
| 6518 | COI | U |
| 6629 | COI | U |
| 6719 | COI | U |
| 7109 | COI | U |
| 7385 | COI | U |
| 7768 | COII | U |
| 7805 | COII | U |
| 8473 | ATPase8 | U |
| 9070 | ATPase6 | U |
| 9266 | COIII | U |
| 9477 | COIII | U |
| 9667 | COIII | U |
| 10506 | ND4L | U |
| 10876 | ND4 | U |
| 10907 | ND4 | U |
| 10927 | ND4 | U |
| 11197 | ND4 | U |
| 11332 | ND4 | U |
| 11732 | ND4 | U |
| 12346 | ND5 | U |
| 12557 | ND5 | U |
| 12618 | ND5 | U |
| 13020 | ND5 | U |
| 13617 | ND5 | U |
| 13637 | ND5 | U |
| 14139 | ND5 | U |
| 14179 | ND6 | U |
| 14182 | ND6 | U |
| 14620 | ND6 | U |
| 14793 | CYT B | U |
| 14866 | CYT B | U |
| 15191 | CYT B | U |
| 15218 | CYT B | U |
| 15454 | CYT B | U |
| 15693 | CYT B | U |
| 15907 | tRNA-T | U |
| 16051 | D-LOOP | U |
| 16256 | D-LOOP | U |
| 16270 | D-LOOP | U |
| 16399 | D-LOOP | U |
| 4580 | ND2 | V |
| 15904 | tRNA-T | V |
| 194 | D-LOOP | W |
| 1243 | 12S rRNA | W |
| 1406 | 12S rRNA | W |
| 3505 | ND1 | W |
| 6528 | COI | W |
| 8994 | ATPase6 | W |
| 10097 | ND3 | W |
| 11674 | ND4 | W |
| 11947 | ND4 | W |
| 12414 | ND5 | W |
| 13263 | ND5 | W |
| 15775 | CYT B | W |
| 15884 | CYT B | W |
| 16292 | D-LOOP | W |
| 225 | D-LOOP | X |
| 226 | D-LOOP | X |
| 6371 | COI | X |
| 8393 | ATPase8 | X |
| 8705 | ATPase6 | X |
| 14470 | ND6 | X |
| 15927 | tRNA-T | X |
| −73 | D-LOOP | not present |
| | | in [H] |
| −7028 | COI | not present |
| | | in [H] |
| −11698 | ND4 | not present |
| | | in [H] |
| and 14766, | CYT B | not present |
| | | in [H] |

and wherein a mitochondrial single nucleotide polymorphism that is haplogroup-associated is, for haplogroup A, B, C, D, E, H, I, J, K, L1, L2, L3, T, U, W or X, located at a nucleotide that corresponds to a nucleotide position of SEQ ID NO:1 that is selected from:

| Nucleotide | | |
|---|---|---|
| Position | Gene | Haplogroup |
| 16362 | D-LOOP | A, C, D, E, |
| | | H, L3 |
| 12705 | ND5 | A, C, D, E, |
| | | I, L1, L2, |
| | | L3, W, X |
| 1888 | 16S rRNA | A, C, T |
| 13708 | ND5 | A, J, X |
| 8027 | COII | A, L1 |
| 153 | D-LOOP | A, X |
| 207 | D-LOOP | B, I, L2, W |
| 13590 | ND5 | B, L2 |
| 9449 | COIII | B, L3 |
| 499 | D-LOOP | B, U |
| 16325 | D-LOOP | C, D |
| 10400 | ND3 | C, D, E |
| 14783 | CYT B | C, D, E |
| 10398 | ND3 | C, D, E, I, |
| | | J, K, L1, |
| | | L2, L3 |
| 15043 | CYT B | C, D, E, |
| | | I, T |
| 489 | D-LOOP | C, D, E, J |
| 8701 | ATPase6 | C, D, E, L1, |
| | | L2, L3 |
| 9540 | COIII | C, D, E, L1, |
| | | L2, L3 |
| 10873 | ND4 | C, D, E, L1, |
| | | L2, L3 |
| 15301 | CYT B | C, D, E, |
| | | L2, L3 |
| 11914 | ND4 | C, K, L1, L2 |
| 16298 | D-LOOP | C, V |
| 3010 | 16S rRNA | D, H, J, U |
| 16311 | D-LOOP | H, K, L1, L3 |
| 93 | D-LOOP | H, L1 |
| 16304 | D-LOOP | H, T |
| 16291 | D-LOOP | H, U |
| 16356 | D-LOOP | H, U |
| 16482 | D-LOOP | H, U |
| 15924 | tRNA-T | I, K, U |
| 10915 | ND4 | I, L1 |
| 204 | D-LOOP | I, L2, W |
| 8251 | COII | I, W |
| 1719 | 16S rRNA | I, X |
| 14798 | CYT B | J, K |
| 15257 | CYT B | J, K |
| 5460 | ND2 | J, L1, W |
| 185 | D-LOOP | J, L3 |
| 11002 | ND4 | J, L3 |
| 4216 | ND1 | J, T |
| 11251 | ND4 | J, T |
| 15452 | CYT B | J, T |
| 16126 | D-LOOP | J, T |
| 9548 | COIII | J, U |
| 13934 | ND5 | J, U |
| 5231 | ND2 | K, L1 |
| 709 | 12S rRNA | K, L1, T, W |
| 1811 | 16S rRNA | K, U |
| 11467 | ND4 | K, U |
| 12308 | tRNA-L | K, U |
| 12372 | ND5 | K, U |
| 182 | D-LOOP | L1, L2 |
| 198 | D-LOOP | L1, L2 |
| 769 | 12S rRNA | L1, L2 |
| 1018 | 12S rRNA | L1, L2 |
| 3594 | ND1 | L1, L2 |
| 4104 | ND1 | L1, L2 |
| 7256 | COI | L1, L2 |
| 7521 | tRNA-D | L1, L2 |
| 13650 | ND5 | L1, L2 |
| 16278 | D-LOOP | L1, L2, L3, X |
| 189 | D-LOOP | L1, L3 |
| 2352 | 16S rRNA | L1, L3 |
| 13105 | ND5 | L1, L3 |
| 5046 | ND2 | L1, W |
| 6152 | COI | L2, U |
| 15784 | CYT B | L2, U, W |
| 5147 | ND2 | L3, T |
| 6221 | COI | L3, X |
| 5426 | ND2 | T, U |
| 13734 | ND5 | T, U |
| and 13966. | ND5 | T, X |

In certain other embodiments the mitochondrial single nucleotide polymorphism that is associated with a mitochondrial haplogroup is present in members of only one haplogroup and is a haplogroup-specific polymorphism as just described that is present in members of only one haplogroup

According to another embodiment of the invention there is provided a method for determining the mitochondrial haplogroup subgroup of a subject, comprising determining, in a biological sample comprising mitochondrial DNA from a subject, the presence or absence of at least one mitochondrial single nucleotide polymorphism that is associated with a mitochondrial haplogroup subgroup. In certain other embodiments the mitochondrial haplogroup is haplogroup K, U, J, T, W, I, H, V, X, L1, L2 or L3. In certain other embodiments at least one mitochondrial single nucleotide polymorphism that is associated with a mitochondrial haplogroup subgroup is a mitochondrial single nucleotide polymorphism located at a nucleotide that corresponds to a nucleotide position of SEQ ID NO:1 that is selected from the group consisting of position 3010, 16162, 16189, 16304, 1811, 3197, 9477, 14793, 16256, 13617, 16270, 7768, 14182, 3480, 9055, 9698, 10550, 11299, 14167, 14798, 16224, 16311, 1189, 10398, 497, 11470, 11914, 15924, 3010, 10398, 12612, 13798, 16069, 295, 489, 228, 462, 16193, 709, 1888, 4917, 8697, 10463, 13368, 14905, 15607, 15928, 16189, 16294, 5426, 6489, 11812, 15043, 16298, 12633, 16163 and 16186.

In another embodiment the invention provides a method for determining a mitochondrial haplogroup subgroup of a subject, comprising determining, in a biological sample comprising mitochondrial DNA from a subject of known mitochondrial haplogroup selected from haplogroups K, U, X, I, J, T, L1, L2 and L3, the presence or absence of a set comprising a plurality of single nucleotide polymorphisms wherein each polymorphism is located at a nucleotide that corresponds to a nucleotide position of SEQ ID NO:1, the set selected from the group consisting of a first haplogroup K subgroup comprising a polymorphism at positions 1147, 12308, 12372, 1811, 3480, 9055, 9698, 10550, 11299, 14167, 14798, 16224, 16311 and 709; a second haplogroup K subgroup comprising a polymorphism at positions 1147, 12308, 12372, 1811, 3480, 9055, 9698, 10550, 11299, 14167, 14798, 16224, 16311, 1189 and 10398; a third haplogroup K subgroup comprising a polymorphism at positions 1147, 12308, 12372, 1811, 3480, 9055, 9698, 10550, 11299, 14167, 14798, 16224, 16311, 1189, 10398 and 497; a fourth haplogroup K subgroup comprising a polymorphism at positions 1147, 12308, 12372, 1811, 3480, 9055, 9698, 10550, 11299, 14167, 14798, 16224, 16311, 1189, 10398, 497 and 11914; a fifth haplogroup K subgroup comprising a polymorphism at positions 1147, 12308, 12372, 1811, 3480, 9055, 9698, 10550, 11299, 14167, 14798, 16224, 16311, 1189, 10398, 497, 11914, 11470 and 15924; a sixth haplogroup K subgroup comprising a polymorphism at positions 1147, 12308, 12372, 1811, 3480, 9055, 9698, 10550, 11299, 14167, 14798, 16224, 16311, 1189, 10398, 497; 11914, 11470, 15924, 12978 and 12954; a seventh haplogroup K subgroup comprising a polymorphism at positions 1147, 12308, 12372, 1811, 3480, 9055, 9698, 10550, 11299, 14167, 14798, 16224, 16311, 1189, 10398, 497; 11914, 11470, 15924, 12978, 12954 and 114; a first haplogroup U subgroup comprising a polymorphism at positions 11467, 12308, 12372 and 1811; a second haplogroup U subgroup comprising a polymorphism at positions 11467, 12308, 12372, 3197, 9477, 13617, 16270; a third haplogroup U subgroup comprising a polymorphism at positions 11467, 12308, 12372, 3197, 9477, 13617, 16270, 7768 and 14182; a fourth haplogroup U subgroup comprising a polymorphism at positions 11467, 12308, 12372, 3197, 9477, 13617, 16270, 14793 and 16256; a fifth haplogroup U subgroup comprising a polymorphism at positions 11467, 12308, 12372, 3197, 9477, 13617, 16270, 14793, 16256 and 15218; a first haplogroup X subgroup comprising a polymorphism at positions 12705, 16223, 1719, 6221, 6371, 13966, 14470, 16278 and 225; a second haplogroup X subgroup comprising a polymorphism at positions 12705, 16223, 1719, 6221, 6371, 13966, 14470, 16278, 225 and 226; a first haplogroup I subgroup comprising a polymorphism at positions 12705, 16223, 1719, 10238, 10398, 12501, 13780 and 15043; a second haplogroup I subgroup comprising a polymorphism at positions 12705, 16223, 1719, 10238, 10398, 12501, 13780, 15043, 250, 4529, 10034, 15924 and 16391; a first haplogroup J subgroup comprising a polymorphism at positions 4216, 11251, 15452, 3010, 10398, 12612, 13708, 16069 and 16126; a second haplogroup J subgroup comprising a polymorphism at positions 4216, 11251, 15452, 3010, 10398, 12612, 13708, 16069, 16126, 295 and 489; a third haplogroup J subgroup comprising a polymorphism at positions 4216, 11251, 15452, 3010, 10398, 12612, 13708, 16069, 16126, 295, 489 and 15257; a fifth haplogroup J subgroup comprising a polymorphism at positions 4216, 11251, 15452, 3010, 10398, 12612, 13708, 16069, 16126, 295, 489 and 462; a sixth haplogroup J subgroup comprising a polymorphism at positions 4216, 11251, 15452, 3010, 10398, 12612, 13708, 16069, 16126, 295, 489, 462 and 228; a first haplogroup T subgroup comprising a polymorphism at positions 709, 1888, 4917, 8697, 10463, 13368, 14905, 15607, 15928, 16126, 16294 and 12633; a second haplogroup T subgroup comprising a polymorphism at positions 709, 1888, 4917, 8697, 10463, 13368, 14905, 15607, 15928, 16126, 16294, 12633, 16163 and 16186; a third haplogroup T subgroup comprising a polymorphism at positions 709, 1888, 4917, 8697, 10463, 13368, 14905, 15607, 15928, 16126, 16294, 11812, 14233 and 16296; a fourth haplogroup T subgroup comprising a polymorphism at positions 709, 1888, 4917, 8697, 10463, 13368, 14905, 15607, 15928, 16126, 16294, 11812, 14233, 16296, 930, 5147 and 16304; a fifth haplogroup T subgroup comprising a polymorphism at positions 709, 1888, 4917, 8697, 10463, 13368, 14905, 15607, 15928, 16126, 16294, 11812, 14233, 16296, 5426, 6489 and 15043; a first haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187 and 16311; a second haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311 and 182; a third haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 8027 and 16294; a fourth haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 357, 709, 710, 1738, 2352, 2768, 3308, 3693, 5036, 5393, 5655, 6548, 6827, 6989, 7867, 8248, 12519, 13880, 14203, 15115, 16126 and 16264; a fifth haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 357, 709, 710, 1738, 2352, 2768, 3308, 3693, 5036, 5393, 5655, 6548, 6827, 6989, 7867, 8248, 12519, 13880, 14203, 15115, 16126, 16264 and 14769; a first haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278 and 16390; a second haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390 and 182; a third haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784 and 16294; a fourth haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784, 16294 and 16309; a fifth haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784, 16294, 16309, 3918, 5285, 15244, 15629; a first haplogroup L3 subgroup comprising a polymorphism at positions 8701, 9540, 10398, 10873, 12705, 16223, 15301; a second haplogroup L3 subgroup comprising a polymorphism at positions 8701, 9540, 10398, 10873, 12705, 16223, 15301, 13105; a third haplogroup L3 subgroup comprising a polymorphism at positions 8701, 9540, 10398, 10873, 12705, 16223, 15301, 13105, 3450, 5773, 6221, 9449, 10089, 10373, 13914, 15311, 15824, 15944, 16124, 16278 and 16362; a fourth haplogroup L3 subgroup comprising a polymorphism at positions 8701, 9540, 10398, 10873, 12705, 16223, 15301, 489, 10400, 14783 and 15043; a fifth haplogroup L3 subgroup comprising a polymorphism at positions 8701, 9540, 10398, 10873, 12705, 16223, 15301, 489, 10400, 14783, 15043, 2092, 3010, 4883, 5178, 6578, 14668 and 16325; a sixth haplogroup L3 subgroup comprising a polymorphism at positions 8701, 9540, 10398, 10873, 12705, 16223, 15301, 489, 10400, 14783, 15043 and 16362; a seventh haplogroup L3 subgroup comprising a polymorphism at positions 8701, 9540, 10398, 10873, 12705, 16223, 15301, 489, 10400, 14783, 15043, 4715, 7196, 8584 and 16298; an eighth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325 and 16327; a ninth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325, 16327, 289 and 290; and a tenth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325, 16327, 289, 290 and 15930.

In another embodiment the invention provides a method for determining the genetic relationship between two subjects, comprising determining, in each of a first biological sample comprising mitochondrial DNA from a first subject and a second biological sample comprising mitochondrial DNA from a second subject, the presence or absence of at least one mitochondrial single nucleotide polymorphism, wherein either (i) the presence of at least one mitochondrial single nucleotide polymorphism in both of said first and second biological samples, or (ii) the absence of at least one mitochondrial single nucleotide polymorphism from both of said first and second biological samples, indicates a genetic relationship between the subjects, and therefrom determining the genetic relationship between the subjects. In certain embodiments at least one mitochondrial single nucleotide polymorphism is associated with a mitochondrial haplogroup that is haplogroup A, B, C, D, E, H, I, J, K, L1, L2, L3, T, U, V, W or X. In certain further embodiments at least one mitochondrial single nucleotide polymorphism is a haplogroup-specific polymorphism as described above.

The invention also provides, in other embodiments, a method for determining the genetic relationship between (i) an unknown source or biological subject from which an unidentified sample is obtained, and (ii) a known source or biological subject from an identified sample is obtained, comprising determining the presence or absence of at least one mitochondrial single nucleotide polymorphism, in each of a first biological sample derived from an unknown subject or biological source and a second biological sample derived from a known subject or biological source, wherein said first and second biological samples each comprise mitochondrial DNA, wherein either (i) the presence of at least one mitochondrial single nucleotide polymorphism in both of said first and second biological samples, or (ii) the absence of at least one mitochondrial single nucleotide polymorphism from both of said first and second biological samples, indicates a genetic relationship between the subjects, and therefrom determining the genetic relationship between the biological samples.

Turning to another embodiment of the present invention, there is provided a method of determining the presence of or the risk for having a disease associated with a mitochondrial DNA single nucleotide polymorphism, comprising (a) identifying at least one haplogroup-associated mitochondrial DNA single nucleotide polymorphism in a biological sample comprising mitochondrial DNA from a subject suspected of having or being at risk for having a disease associated with a mitochondrial DNA single nucleotide polymorphism; and (b) identifying in said sample at least one disease associated mitochondrial DNA single nucleotide polymorphism that is not a haplogroup-associated mitochondrial DNA single nucleotide polymorphism, and therefrom determining the presence or risk of disease. In certain other embodiments the disease associated mitochondrial DNA single nucleotide polymorphism that is not a haplogroup-associated mitochondrial DNA single nucleotide polymorphism is an Alzheimer's disease-associated polymorphism, and in certain other embodiments the disease associated mitochondrial DNA single nucleotide polymorphism that is not a haplogroup-associated mitochondrial DNA single nucleotide polymorphism is a type 2 diabetes-associated polymorphism.

These and other aspects of the present invention will become apparent upon reference to the following detailed description and attached drawings. All references disclosed herein are hereby incorporated by reference in their entireties as if each was incorporated individually

### BRIEF DESCRIPTION OF THE DRAWINGS

FIG. 1 provides a phylogenetic tree of European mtDNA haplotypes.

FIG. 2 provides a phylogenetic tree of African mtDNA haplotypes.

FIGS. 3-6 show reduced median network of the specified mtDNA haplogroups. For reduced median network analysis, see, e.g., Bandelt et al., 1995 Genetics 141:743-753.

FIG. 3 shows a reduced median network of European mtDNA haplogroups.

FIG. 4 shows a reduced median network of European H and V mtDNA haplogroups.

FIG. 5 shows a reduced median network of African mtDNA haplogroups.

FIG. 6 shows a reduced median network of Asian mtDNA haplogroups.

### DETAILED DESCRIPTION OF THE INVENTION

The present invention provides improved compositions and methods for identifying individuals, subpopulations and populations by determination of mtDNA haplogroup, genealogic, forensic, and related genetic relationships. As described herein, surprising diversity in mtDNA sequences permits expanded definition of mitochondrial polymorphism and redefinition of mtDNA haplogroups and subgroups at a level of refinement not previously recognized. The invention thus exploits the high mutation rate of mitochondrial DNA (mtDNA) to identify individuals, subpopulations and/or populations on the basis of specific mutations associated with particular characteristics such as race, genealogy and/or the presence of, or risk for having, certain diseases. In addition, mtDNA may be used to identify specific individuals. The present invention is directed generally to compositions and methods for identifying mtDNA mutations and thereby diagnosing the risk for having, or presence of, a disease. The invention also permits determination of other characteristics such as genealogy, population, race or ethnic group. In addition, the methods of the present invention are directed to identifying genetic and familial relationships between subjects or biological sources of mitochondrial DNA samples for a variety of purposes including, for instance, maternity testing, forensic studies, genetic counseling and genealogical analysis, and the like.

Biological samples may be provided by obtaining a blood sample, biopsy specimen, tissue explant, organ culture or any other tissue or cell preparation from a subject or a biological source, and in most preferred embodiments of the invention the biological sample comprises mtDNA. The subject or biological source may be a human or another biological organism, including a genetically engineered organism, such as a non-human animal, a plant, a unicellular organism or a multicellular organism or mitochondria prepared therefrom. The subject or biological source may also be a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines (see, e.g., U.S. Pat. No. 5,888,498), differentiated or differentiatable cell lines, transformed cell lines and the like. A biological sample may, for example, be derived from a recombinant cell line or from a transgenic animal.

In certain embodiments of the invention, a subject or biological source may be infected with a microorganism such as a DNA virus, a retrovirus, a mycoplasma or a bacterium. In particular embodiments of the invention, for instance, those that relate to forensic sciences, a subject or biological source may provide material comprising mitochondrial DNA that is found at a crime scene or that may be otherwise associated with a person (including, for example, a criminal suspect), place or thing with which a suspect may have come into contact, for use as evidence. In certain related embodiments a biological sample may be derived from an unknown source or biological subject to provide an unidentified sample, which may then be characterized using the compositions and methods described herein. In certain other related embodiments such characterization may be used to determine a mitochondrial genetic relationship between the unknown source or biological subject and one or more of a particular species, a mitochondrial haplogroup, a mitochondrial haplogroup subgroup or a known source or biological subject having at least one mitochondrial single nucleotide polymorphism as provided herein, for instance, to identify the biological subject and/or to determine a genetic relationship between the subject and another individual, population or subpopulation (e.g., a haplogroup, subgroup or family).

In certain embodiments or the invention, the subject or biological source may be suspected of having or being at risk of having a disease associated with altered mitochondrial function, (e.g., Alzheimer's Disease, type 2 diabetes mellitus), and in certain embodiments of the invention, the subject or biological source may be known to be free of a risk for, or presence of, such a disease, or the risk or presence of a disease may not be known. In other embodiments of the invention, the subject or source may be suspected of being involved in an illegal activity. A subject or sample may be suspected of being genetically related to a specific individual or of belonging to a certain genealogical lineage, race or ethnic group. In other embodiments, the subject or source may not be suspected of being involved in an illegal activity or of being genetically related to a specific individual or of belonging to a certain genealogical lineage, race or ethnic group.

For example, and according to non-limiting theory, in certain embodiments it may be desirable to use as a subject or biological source a control individual selected according to criteria that will be apparent to a person having ordinary skill in the art based on one or more variables which may require normalization, typically an age- and/or sex-matched individual, a healthy individual or an individual appropriate as a control for a subject suspected of having or being at risk for a particular disease It may also be desirable to use as a subject or biological source a control individual for comparison purposes for maternity or forensic tests. Those having ordinary skill in the art are thus familiar with the design and selection of appropriate controls for different particular purposes. For instance, in certain embodiments it may be desirable to identify such a control individual who is believed to be free of a particular disease, and in certain other embodiments, a control individual may share a mitochondrial genetic relationship to a subject suspected of having a particular disease, such as the mother or sibling of the subject.

According to the present invention there is provided the unexpected discovery that determination of unprecedented polymorphism in mitochondrial DNA permits refinement of the assignment of individuals to particular mitochondrial haplogroups and haplogroup subgroups as provided herein. Additionally, the present invention exploits the surprising discoveries that in many cases, the haplogroup and/or haplogroup subgroup to which an individual belongs may not be determinative of a presence of a disease or of a risk for having a disease. Rather, as provided by the present disclosure, the improved ability to identify mitochondrial single nucleotide polymorphisms that are associated with particular haplogroups and/or haplogroup subgroups as described herein further permits identification of additional mitochondrial single nucleotide polymorphisms.

As described in greater detail herein, such additional polymorphisms, which are not definitive for a particular haplogroup and/or haplogroup subgroup, are useful correlates for other purposes, for example, in the identification of unique individuals (e.g., in forensics) and/or for determination of an individual's disease predisposition. Thus, in certain embodiments the present invention provides an improved system for distinguishing individuals belonging to the same mitochondrial haplogroup on the basis of particular mitochondrial DNA polymorphisms described herein. For instance, individuals of a common maternal lineage would fall into a common haplogroup according to standard mitochondrial genotyping methodologies without there being a basis for further differentiation, while the present invention provides one or more mitochondrial single nucleotide polymorphisms that are unique to each individual within a haplogroup (or haplogroup subgroup) thereby permitting such differentiation. These and other embodiments are also described in greater detail below.

In certain preferred embodiments it may be desirable to determine whether the subject, patient or biological source falls within clinical parameters indicative of a disease such as, but not limited to, Alzheimer's disease (AD) or type 2 diabetes mellitus (type 2 DM). Signs and symptoms of AD accepted by those skilled in the art may be used to so designate a subject or biological source, for example, clinical signs referred to in McKhann et al. (Neurology 34:939, 1984, National Institute of Neurology, Communicative Disorders and Stroke and Alzheimer's Disease and Related Disorders Association Criteria of Probable AD, NINCDS-ADRDA) and references cited therein, or other means known in the art for diagnosing AD. Similarly, signs and symptoms of type 2 diabetes mellitus accepted by those skilled in the art may be used to so designate a subject or biological source, for example, clinical signs referred to in Gavin et al. (Diabetes Care 22 (Suppl. 1):55-519, 1999, American Diabetes Association Expert Committee on the Diagnosis and Classification of Diabetes Mellitus) and references cited therein, or other means known in the art for diagnosis of type 2 diabetes mellitus. Also, by way of illustration and not limitation, accepted criteria such as particular clinical signs and symptoms (or ranges or combinations thereof) will be known to those familiar with the art for any of the other diseases associated with altered mitochondrial function as provided herein. (See, e.g., Chinnery et al., 1999 J. Med. Genet. 36:425)

For example, according to certain embodiments the present invention provides a method for determining the risk for having, or presence of, a malignant condition in a subject. A malignant condition in a subject, as used herein, refers to the presence of dysplastic, cancerous and/or transformed cells in the subject, including, for example neoplastic, tumor, non-contact inhibited or oncogenically transformed cells, or the like. By way of illustration and not limitation, in the context of the present invention a malignant condition may refer further to the presence in a subject of cancer cells such as colorectal cancer, lung cancer, bladder cancer, or head and neck tumors, as have been described in the context of somatic mtDNA sequence variations distinct from the mitochondrial single nucleotide polymorphisms described herein (cf, Fliss et al., 1999 Science 287:2017; Polyak et al., 1998 Nat. Genet. 20:291).

In other preferred embodiments, it may be useful to combine the present invention with other procedures for identifying an individual, including methods of analyzing either mtDNA or genomic DNA (e.g., extramitochondrial DNA, i.e., nuclear chromosomal or episomal DNA) such as, for example, RFLP analysis, allele specific oligonucleotide analysis or any other technique for DNA analysis known in the art, including those described above.

As noted above, a biological sample for use according to the most highly preferred embodiments of the present invention contains mtDNA as provided herein, and may comprise any source of mitochondrial DNA, including any tissue or cell preparation in which mitochondrially derived nucleic acids (e.g., mtDNA) are present. In addition, a source or biological sample comprising mitochondrial DNA may include a source of mtDNA wherein cells or tissues are not present. Biological samples may therefore contain live cells, or dead cells or no cells. Compositions and methods useful for obtaining and detecting mtDNA are provided, for example, in U.S. Pat. Nos. 5,565,323 and 5,840,493. Biological samples may thus be provided by obtaining a sample of blood, hair, scalp, skin or other epithelial cells, bone, saliva, mucous or other secretion, semen, or other forensic sample, biopsy specimen, tissue explant, organ culture or any other tissue, cell preparation or non-cell preparation from a subject or a biological source as provided herein. Thus, for example, in certain specific embodiments, biological samples of the invention may include mtDNA isolated at a crime scene or from another source of forensic evidence. In certain other embodiments, biological samples may include mtDNA isolated from archaeological sites or from human or animal remains.

Any mtDNA sequence or portion of a mutated mtDNA (e.g., mtDNA that contains at least one single nucleotide polymorphism as provided herein, including mtDNA that contains a plurality of such single nucleotide polymorphisms) sequence that corresponds to the human mtDNA sequence disclosed by Anderson et al. (SEQ ID NO:1, 1981 Nature 290:457; see also Marzuki et al., 1991 Human Genet. 88:139) and revised according to Andrews et al. (1999 Nature Genetics 23:147), or a portion thereof or several portions thereof, may be useful in these embodiments of the invention. Examples of human mtDNA point mutations derived from specific mtDNA sequence regions that are useful in certain embodiments of the invention are disclosed, according to the nucleotide positions at which wildtype and mutant mtDNA differ, in Tables 1 and 2. Those familiar with the art will recognize the established convention for naming regions of the circular mtDNA genome according to the D-loop and the several mtDNA gene loci, including the mitochondrial rRNA genes, the mitochondrial tRNA genes, the mitochondrial NADH dehydrogenase genes, the mitochondrial cytochrome c oxidase genes, the mitochondrial ATP synthase genes and the mitochondrial cytochrome b gene, and the corresponding nucleotide position numbers of SEQ ID NO:1 that are spanned by each of these regions (see, e.g., Scheffler, Mitochondria, 1999 John Wiley & Sons, pages 48-140, and references cited therein; see also “Mitomap” at http://www.gen.emory.edu/mitomap.html). Thus, for example, Table 1 shows mitochondrial single nucleotide polymorphisms that include polymorphisms which have been correlated with Alzheimer's disease, as disclosed in co-pending U.S. patent application Ser. No. 09/551,941 which is hereby incorporated by reference, and Table 2 shows mitochondrial single nucleotide polymorphisms that include polymorphisms which have been correlated with type 2 diabetes in certain mitochondrial haplogroups, as disclosed in the co-pending U.S. Patent application No. 60/333,448, hereby incorporated by reference. Full-length mtDNA sequences from 560 human subjects are disclosed herein in the Sequence Listing and are set forth at SEQ ID NOS:2-561.

TABLE 1
MTDNA SINGLE NUCLEOTIDE POLYMORPHISMS
ASSOCIATED WITH AD

| | | | Nucl. | | |
|---|---|---|---|---|---|
| | Nucleotide | Nucleotide | Substi- | # | Haplo- |
| Gene | Position | (CRS) | tution | Samples | group |
| D-LOOP | 72 | T | C | 1 | H |
| D-LOOP | 114 | C | T | 1 | K |
| D-LOOP | 146 | T | C | 2 | U, H |
| D-LOOP | 185 | G | A | 1 | J |
| D-LOOP | 189 | A | G | 1 | K, W |
| D-LOOP | 199 | T | C | 1 | I |
| D-LOOP | 204 | T | C | 1 | W |
| D-LOOP | 207 | G | A | 2 | W, I |
| D-LOOP | 228 | G | A | 1 | J |
| D-LOOP | 236 | T | C | 1 | H |
| D-LOOP | 239 | T | C | 2 | H |
| D-LOOP | 456 | C | T | 1 | H |
| D-LOOP | 462 | C | T | 2 | J |
| D-LOOP | 482 | T | C | 1 | J |
| D-LOOP | 489 | T | C | 2 | J |
| D-LOOP | 497 | C | T | 1 | K, K |
| D-LOOP | 500 | C | G | 5 | H, W, J |
| D-LOOP | 516 | C | T | 1 | U |
| D-LOOP | 522 | C | DEL | 1 | H |
| D-LOOP | 523 | A | DEL | 1 | H |
| D-LOOP | 547 | A | T | 1 | I |
| 12S RRNA | 593 | T | C | 1 | K |
| 12S RRNA | 669 | T | C | 1 | I |
| 12S RRNA | 960 | C | DEL | 1 | U |
| 12S RRNA | 1007 | G | A | 1 | J |
| 12S RRNA | 1243 | T | C | 1 | W |
| 12S RRNA | 1393 | G | A | 1 | H |
| 16S RRNA | 1719 | G | A | 1 | H, I |
| 16S RRNA | 1809 | T | C | 1 | U |
| 16S RRNA | 2352 | T | C | 1 | H |
| 16S RRNA | 2483 | T | C | 1 | K |
| 16S RRNA | 2702 | G | A | 1 | I |
| 16S RRNA | 2851 | A | G | 1 | H |
| 16S RRNA | 3197 | T | C | 1 | U |
| ND1 | 3333 | C | T | 1 | H |
| ND1 | 3336 | T | C | 1 | I |
| ND1 | 3348 | A | G | 1 | U |
| ND1 | 3394 | T | C | 1 | J |
| ND1 | 3398 | T | C | 1 | I |
| ND1 | 3423 | G | T | 1 | J |
| ND1 | 3505 | A | G | 1 | W |
| ND1 | 3559 | C | T | 1 | H |
| ND1 | 3915 | G | A | 2 | H |
| ND1 | 3992 | C | T | 1 | H |
| ND1 | 4024 | A | G | 1 | H |
| ND1 | 4095 | C | T | 1 | H |
| ND1 | 4216 | T | C | 3 | T, J |
| TRNA-Q | 4336 | T | C | 1 | H |
| ND2 | 4529 | A | T | 1 | I |
| ND2 | 4727 | A | G | 2 | H |
| ND2 | 4793 | A | G | 1 | H |
| ND2 | 4917 | A | G | 1 | T |
| ND2 | 4991 | G | A | 1 | H |
| ND2 | 5004 | T | C | 2 | H, W |
| ND2 | 5046 | G | A | 1 | W |
| ND2 | 5228 | C | G | 1 | H |
| ND2 | 5315 | A | G | 1 | I |
| ND2 | 5418 | T | G | 1 | J |
| ND2 | 5426 | T | C | 1 | T |
| ND2 | 5460 | G | A | 3 | H, W |
| ND2 | 5461 | C | G | 1 | J |
| TRNA-W | 5516 | A | G | 1 | H |
| TRNA-W | 5554 | C | A | 1 | U |
| TRNA-A | 5634 | A | G | 1 | H |
| TRNA-A/ | 5656 | A | G | 1 | U |
| TRNA-N | | | | | |
| TRNA-C | 5773 | G | A | 1 | J |
| CO1 | 6182 | G | A | 1 | U |
| CO1 | 6221 | T | C | 1 | H |
| CO1 | 6341 | C | T | 1 | U |
| CO1 | 6367 | T | C | 1 | K |
| CO1 | 6371 | C | T | 1 | H |
| CO1 | 6489 | C | A | 1 | T |
| CO1 | 7184 | A | G | 1 | J |
| CO1 | 7325 | A | G | 1 | H |
| CO2 | 7621 | T | C | 1 | K |
| CO2 | 7768 | A | G | 1 | U |
| CO2 | 7787 | C | T | 1 | H |
| CO2 | 7789 | G | A | 1 | J |
| CO2 | 7864 | C | T | 1 | W |
| CO2 | 7895 | G | A | 1 | U |
| CO2 | 7963 | A | G | 1 | J |
| CO2 | 8149 | A | G | 1 | H |
| CO2 | 8251 | G | A | 2 | W, I |
| CO2 | 8269 | G | A | 1 | H |
| CO2/ | 8276-8284 | | DEL | 1 | T |
| TRNA-K | | | | | |
| ATPAse 8 | 8470 | A | G | 1 | H |
| ATPASE 8 | 8485 | G | A | 1 | I |
| ATPASE 8 | 8508 | A | G | 1 | I |
| ATPASE 6 | 8602 | T | C | 1 | H |
| ATPASE6 | 8697 | G | A | 1 | T |
| ATPASE 6 | 8752 | A | G | 1 | H |
| ATPASE 6 | 8901 | A | G | 1 | I |
| ATPASE 6 | 8994 | G | A | 1 | W |
| ATPASE 6 | 9123 | G | A | 1 | H |
| CO3 | 9254 | A | G | 1 | H |
| CO3 | 9362 | A | G | 1 | H |
| CO3 | 9380 | G | A | 2 | H |
| CO3 | 9477 | G | A | 1 | U |
| CO3 | 9554 | G | A | 1 | H |
| CO3 | 9708 | T | C | 1 | H |
| CO3 | 9804 | G | A | 1 | H |
| CO3 | 9861 | T | C | 1 | H |
| TRNA-G | 10034 | T | C | 1 | I |
| TRNA-G | 10044 | A | G | 1 | H |
| ND3 | 10238 | T | C | 2 | I |
| TRNA-R | 10463 | T | C | 2 | T, J |
| ND4L | 10589 | G | A | 1 | H |
| ND4 | 10978 | A | G | 1 | K |
| ND4 | 11065 | A | G | 1 | I |
| ND4 | 11251 | A | G | 1 | J |
| ND4 | 11253 | T | C | 1 | H |
| ND4 | 11272 | A | G | 1 | U |
| ND4 | 11470 | A | G | 2 | K |
| ND4 | 11527 | C | T | 1 | J |
| ND4 | 11611 | G | A | 1 | H |
| ND4 | 11674 | C | T | 1 | W |
| ND4 | 11812 | A | G | 1 | T |
| ND4 | 11914 | G | A | 2 | K |
| ND4 | 11947 | A | G | 1 | W |
| ND5 | 12414 | T | C | 1 | W |
| ND5 | 12501 | G | A | 2 | I |
| ND5 | 12609 | T | C | 1 | U |
| ND5 | 12705 | C | T | 4 | H, W, I |
| ND5 | 12954 | T | C | 1 | K |
| ND5 | 13111 | T | C | 1 | H |
| ND5 | 13194 | G | A | 2 | H, U |
| ND5 | 13212 | C | T | 1 | H |
| ND5 | 13368 | G | A | 1 | T |
| ND5 | 13617 | T | C | 1 | U |
| ND5 | 13780 | A | G | 2 | I |
| ND5 | 13966 | A | G | 1 | H |
| ND5 | 14020 | T | C | 1 | T |
| ND5 | 14148 | A | G | 1 | W |
| ND6 | 14178 | T | C | 1 | I |
| ND6 | 14179 | A | G | 1 | U |
| ND6 | 14182 | T | C | 1 | U |
| ND6 | 14212 | T | C | 1 | H |
| ND6 | 14233 | A | G | 1 | T |
| ND6 | 14470 | T | C | 1 | H |
| ND6 | 14582 | A | G | 1 | H |
| CYT.B | 14905 | G | A | 1 | T |
| CYT.B | 15028 | C | A | 1 | T |
| CYT.B | 15043 | G | A | 3 | T, I |
| CYT.B | 15191 | T | C | 1 | U |
| CYT.B | 15299 | T | C | 1 | I |
| CYT.B | 15380 | A | G | 1 | U |
| CYT.B | 15553 | G | A | 1 | H |
| CYT.B | 15607 | A | G | 1 | T |
| CYT.B | 15758 | A | G | 1 | I |
| CYT.B | 15790 | C | T | 1 | U |
| CYT.B | 15808 | A | G | 1 | H |
| CYT.B | 15833 | C | T | 1 | H |
| CYT.B | 15884 | G | C | 1 | W |
| TRNA-T | 15924 | A | G | 3 | K, I |
| TRNA-T | 15928 | G | A | 1 | T |
| D-LOOP | 16069 | C | T | 2 | J |
| D-LOOP | 16086 | T | C | 1 | I |
| D-LOOP | 16093 | T | C | 1 | K |
| D-LOOP | 16126 | T | C | 3 | T, J |
| D-LOOP | 16129 | G | A | 2 | H, I |
| D-LOOP | 16145 | G | A | 1 | I |
| D-LOOP | 16147 | C | A | 1 | I |
| D-LOOP | 16172 | T | C | 1 | U |
| D-LOOP | 16174 | C | T | 1 | U |
| D-LOOP | 16182 | A | C | 1 | T |
| D-LOOP | 16183 | A | C | 4 | T, U, H |
| D-LOOP | 16189 | T | C | 5 | T, U, H |
| D-LOOP | 16192 | C | T | 1 | U |
| D-LOOP | 16193 | C | T | 1 | J |
| D-LOOP | 16223 | C | T | 4 | H, W, I |
| D-LOOP | 16224 | T | C | 3 | K |
| D-LOOP | 16234 | C | T | 1 | K |
| D-LOOP | 16235 | A | G | 1 | J |
| D-LOOP | 16239 | C | T | 1 | H |
| D-LOOP | 16248 | C | T | 1 | I |
| D-LOOP | 16256 | C | T | 1 | J |
| D-LOOP | 16261 | C | T | 1 | H |
| D-LOOP | 16270 | C | T | 1 | U |
| D-LOOP | 16278 | C | T | 2 | U, H |
| D-LOOP | 16290 | C | T | 1 | H |
| D-LOOP | 16292 | C | T | 1 | W |
| D-LOOP | 16293 | A | G | 1 | H |
| D-LOOP | 16294 | C | T | 1 | T |
| D-LOOP | 16298 | T | C | 2 | T, H |
| D-LOOP | 16300 | A | G | 1 | J |
| D-LOOP | 16304 | T | C | 1 | H |
| D-LOOP | 16309 | A | G | 1 | J |
| D-LOOP | 16311 | T | C | 5 | H, K, U |
| D-LOOP | 16320 | C | T | 1 | I |
| D-LOOP | 16355 | C | T | 1 | I |
| D-LOOP | 16362 | T | C | 2 | H |
| D-LOOP | 16391 | G | A | 1 | I |
| D-LOOP | 16482 | A | G | 2 | H |
| D-LOOP | 16524 | A | G | 1 | K |
| D-LOOP | 45 | C | INS | 1 | U |
| D-LOOP | 140 | C | T | 1 | H |
| D-LOOP | 188 | A | G | 2 | J |
| D-LOOP | 291 | A | INS | 1 | U |
| D-LOOP | 455 | T | DEL | 1 | U |
| D-LOOP | 500 | C | G | 2 | H |
| D-LOOP | 513 | G | DEL | 1 | H |
| D-LOOP | 514 | C | DEL | 1 | H |
| D-LOOP | 516 | C | T | 1 | U |
| D-LOOP | 527 | C | G | 2 | K, T |
| D-LOOP | 533 | A | G | 1 | I |
| D-LOOP | 568 | CCC | INS | 1 | I |
| D-LOOP | 710 | G | A | 1 | H |
| 12S RRNA | 749 | A | G | 1 | K |
| 12S RRNA | 960 | C | DEL | 1 | U |
| 12S RRNA | 1508 | C | T | 1 | H |
| 16S RRNA | 1809 | T | C | 1 | U |
| 16S RRNA | 2352 | T | C | 1 | H |
| 16S RRNA | 2833 | A | G | 1 | U |
| ND1 | 3559 | C | T | 1 | X |
| ND1 | 3745 | G | A | 1 | U |
| ND2 | 5237 | G | A | 1 | K |
| ND2 | 5348 | C | T | 1 | H |
| TRNA-W | 5516 | A | G | 1 | H |
| TRNA-W | 5554 | C | A | 1 | U |
| TRNA-C | 5773 | G | A | 1 | H |
| CO1 | 5979 | G | A | 1 | H |
| CO1 | 6182 | G | A | 1 | U |
| CO1 | 6272 | A | G | 1 | H |
| CO1 | 6320 | T | C | 1 | T |
| CO1 | 6341 | C | T | 1 | U |
| CO1 | 6480 | G | A | 2 | H, T |
| CO1 | 6498 | C | G | 1 | U |
| CO1 | 6722 | G | A | 1 | B |
| CO1 | 6845 | C | T | 1 | K |
| CO1 | 6911 | T | C | 1 | U |
| CO2 | 7787 | C | T | 1 | X |
| CO2 | 7830 | G | A | 1 | H |
| CO2 | 7853 | G | A | 1 | T |
| CO2 | 7927 | C | G | 1 | H |
| CO2 | 7978 | C | G | 1 | H |
| CO2 | 7985 | C | G | 2 | H, U |
| CO2 | 8149 | A | G | 1 | H |
| ATPASE 6 | 8865 | G | A | 1 | U |
| ATPASE 6 | 9098 | T | C | 1 | B |
| CO3 | 9129 | C | T | 1 | H |
| CO3 | 9708 | T | C | 1 | H |
| CO3 | 9861 | T | C | 1 | H |
| ND3 | 10154 | A | G | 1 | K |
| ND3 | 10394 | C | T | 1 | H |
| TRNA-R | 10448 | T | C | 1 | H |
| ND4L | 10724 | T | C | 1 | U |
| ND4 | 11272 | A | G | 1 | U |
| ND4 | 11590 | A | G | 1 | H |
| ND4 | 11824 | A | G | 1 | H |
| ND4 | 11893 | A | G | 1 | H |
| TRNA-S | 12217 | A | G | 1 | H |
| ND5 | 12471 | T | C | 1 | H |
| ND5 | 12609 | T | C | 1 | U |
| ND5 | 12738 | T | G | 1 | K |
| ND5 | 13194 | G | A | 1 | U |
| ND5 | 13212 | C | T | 1 | X |
| ND5 | 13351 | C | T | 1 | U |
| ND5 | 13746 | C | T | 1 | H |
| ND5 | 13889 | G | A | 1 | H |
| ND5 | 13943 | C | T | 1 | K |
| ND5 | 14133 | A | G | 1 | H |
| ND6 | 14323 | G | A | 1 | H |
| ND6 | 14512 | T | C | 1 | U |
| TRNA-E | 14684 | C | T | 1 | U |
| CYB | 14869 | G | A | 1 | H |
| CYB | 14978 | A | G | 1 | H |
| CYB | 15295 | C | T | 1 | T |
| CYB | 15380 | A | G | 1 | U |
| CYB | 15734 | G | A | 1 | U |
| CYB | 15790 | C | T | 1 | U |
| TRNA-T | 15947 | A | G | 1 | J |
| D-LOOP | 16136 | T | C | 1 | H |
| D-LOOP | 16168 | C | T | 1 | U |
| D-LOOP | 16174 | C | T | 1 | U |
| D-LOOP | 16209 | T | C | 1 | T |
| D-LOOP | 16219 | A | G | 1 | U |
| D-LOOP | 16239 | C | T | 1 | U |
| D-LOOP | 16260 | C | T | 1 | J |
| D-LOOP | 16263 | T | C | 1 | H |
| D-LOOP | 16295 | C | T | 1 | H |
| D-LOOP | 16343 | A | G | 1 | U |
| D-LOOP | 16524 | A | G | 1 | H |
| D-LOOP | 16526 | G | A | 1 | U |

TABLE 2
MITOCHONDRIAL SNPS AND HAPLOGROUPS IN NIDDM

| | | | NUCL. | | |
|---|---|---|---|---|---|
| | NUCLEOTIDE | NUCLEOTIDE | SUBSTI- | | HAPLO- |
| GENE | POSITION | (CRS) | TUTION | # SAMPLES | GROUP |
| D-LOOP | 62 | G | C | 1 | B |
| D-LOOP | 62 | G | C | 1 | B |
| D-LOOP | 94 | G | A | 1 | A |
| D-LOOP | 94 | G | A | 1 | A |
| D-LOOP | 95 | A | C | 1 | L1 |
| D-LOOP | 114 | C | G | 1 | B |
| D-LOOP | 151 | C | T | 1 | L1 |
| D-LOOP | 159 | T | C | 1 | H |
| D-LOOP | 188 | A | G | 1 | J |
| D-LOOP | 198 | C | T | 1 | L2 |
| D-LOOP | 215 | A | G | 1 | C |
| D-LOOP | 215 | A | G | 1 | C |
| D-LOOP | 257 | A | G | 1 | H |
| D-LOOP | 267 | T | C | 1 | D |
| D-LOOP | 271 | C | T | 1 | B |
| D-LOOP | 288 | A | G | 1 | A |
| D-LOOP | 317 | G | A | 1 | L1 |
| D-LOOP | 317 | G | A | 1 | L1 |
| D-LOOP | 320 | C | T | 1 | B |
| D-LOOP | 343 | C | T | 1 | B |
| D-LOOP | 362 | A | INS | 1 | A |
| D-LOOP | 418 | C | T | 1 | L2 |
| D-LOOP | 453 | T | DEL | 1 | B |
| D-LOOP | 454 | T | DEL | 1 | B |
| D-LOOP | 466 | T | C | 1 | U |
| D-LOOP | 480 | T | C | 1 | H |
| D-LOOP | 481 | C | T | 1 | B |
| D-LOOP | 481 | C | T | 1 | B |
| D-LOOP | 493 | A | G | 1 | C |
| D-LOOP | 568 | C | INS | 1 | D |
| D-LOOP | 568 | C | INS | 1 | H |
| D-LOOP | 568 | C | INS | 1 | T |
| D-LOOP | 568 | CC | INS | 1 | K |
| D-LOOP | 568 | C | INS | 1 | C |
| TRNA-F | 629 | T | C | 1 | A |
| 12S RRNA | 735 | A | G | 1 | H |
| 12S RRNA | 921 | T | C | 1 | L3 |
| 12S RRNA | 956 | C | INS | 1 | B |
| 12S RRNA | 956 | C | INS | 1 | A |
| 12S RRNA | 956 | C | INS | 1 | B |
| 12S RRNA | 960 | C | DEL | 1 | U |
| 12S RRNA | 961 | T | C | 1 | A |
| 12S RRNA | 961 | T | C | 1 | A |
| 12S RRNA | 966 | CC | INS | 1 | A |
| 12S RRNA | 966 | C | INS | 1 | A |
| 12S RRNA | 1002 | C | T | 1 | B |
| 12S RRNA | 1002 | C | T | 1 | B |
| 12S RRNA | 1007 | G | C | 1 | L2 |
| 12S RRNA | 1420 | T | C | 1 | L1 |
| 12S RRNA | 1462 | G | A | 1 | H |
| 16S RRNA | 1766 | T | C | 1 | L1 |
| 16S RRNA | 2145 | G | A | 1 | H |
| 16S RRNA | 2157 | A | INS | 1 | L1 |
| 16S RRNA | 2735 | G | A | 1 | C |
| 16S RRNA | 2755 | A | G | 1 | L1 |
| 16S RRNA | 2863 | T | C | 1 | L1 |
| 16S RRNA | 2903 | T | C | 1 | L3 |
| 16S RRNA | 3203 | A | G | 1 | L3 |
| ND1 | 3308 | T | A | 1 | A |
| ND1 | 3309 | C | INS | 1 | A |
| ND1 | 3311 | C | T | 1 | A |
| ND1 | 3338 | T | C | 1 | L2 |
| ND1 | 3338 | T | C | 1 | B |
| ND1 | 3513 | C | T | 1 | L1 |
| ND1 | 3766 | T | C | 1 | B |
| ND1 | 3766 | T | C | 1 | B |
| ND1 | 3777 | T | C | 1 | L2 |
| ND1 | 3927 | A | G | 1 | L1 |
| ND1 | 3936 | C | A | 1 | L2 |
| ND1 | 4012 | A | G | 1 | B |
| ND1 | 4129 | A | G | 1 | T |
| ND1 | 4167 | C | T | 1 | B |
| ND1 | 4167 | C | T | 1 | B |
| ND1 | 4232 | T | C | 1 | B |
| ND1 | 4242 | C | T | 1 | C |
| TRNA-Q | 4386 | T | C | 1 | H |
| TRNA-Q | 4392 | C | T | 1 | L3 |
| TRNA-M | 4435 | A | G | 1 | D |
| ND2 | 4506 | A | G | 1 | L1 |
| ND2 | 4655 | G | A | 1 | L2 |
| ND2 | 4733 | T | C | 1 | L3 |
| ND2 | 4908 | C | T | 1 | U |
| ND2 | 5156 | A | T | 1 | H |
| ND2 | 5165 | C | T | 1 | A |
| ND2 | 5183 | A | G | 1 | A |
| ND2 | 5237 | G | A | 1 | L1 |
| ND2 | 5372 | A | G | 1 | L3 |
| TRNA-W | 5463 | G | A | 1 | H |
| TRNA-C | 5824 | G | A | 1 | D |
| TRNA-Y/CO1 | 5899 | C | DEL | 1 | B |
| TRNA-Y/CO1 | 5900 | CC | INS | 1 | L1 |
| CO1 | 6026 | G | A | 1 | L2 |
| CO1 | 6164 | C | T | 1 | B |
| CO1 | 6164 | C | T | 1 | L2 |
| CO1 | 6164 | C | T | 1 | B |
| CO1 | 6308 | C | T | 1 | A |
| CO1 | 6308 | C | T | 1 | A |
| CO1 | 6311 | C | T | 1 | L2 |
| CO1 | 6340 | C | T | 1 | H |
| CO1 | 6378 | T | C | 1 | L1 |
| CO1 | 6392 | T | C | 1 | T |
| CO1 | 6620 | T | C | 1 | A |
| CO1 | 6663 | A | G | 1 | L2 |
| CO1 | 6710 | A | G | 1 | K |
| CO1 | 6722 | G | A | 1 | B |
| CO1 | 6932 | A | G | 1 | L3 |
| CO1 | 6951 | G | A | 1 | D |
| CO1 | 6951 | G | A | 1 | K |
| CO1 | 7158 | A | G | 1 | B |
| BO1 | 7158 | A | G | 1 | B |
| CO1 | 7202 | A | G | 1 | L1 |
| CO2 | 7621 | T | C | 1 | H |
| CO2 | 7692 | T | C | 1 | L1 |
| CO2 | 7697 | G | A | 1 | C |
| CO2 | 7702 | G | A | 1 | L2 |
| CO2 | 7853 | G | A | 1 | D |
| CO2 | 7978 | C | G | 1 | L3 |
| CO2 | 7978 | C | G | 1 | L2 |
| CO2 | 7985 | C | G | 1 | L3 |
| CO2 | 7985 | C | G | 1 | L2 |
| CO2 | 8032 | C | T | 1 | A |
| CO2 | 8087 | T | C | 1 | L1 |
| CO2 | 8098 | A | G | 1 | K |
| CO2 | 8119 | T | C | 1 | L2 |
| ATPASE 8 | 8419 | T | C | 1 | L1 |
| ATPASE 8 | 8460 | A | G | 1 | B |
| ATPASE 8 | 8460 | A | G | 1 | B |
| ATPASE 8 | 8478 | C | T | 1 | H |
| ATPASE 8 | 8530 | A | G | 1 | L3 |
| ATPASE 8 | 8557 | G | A | 1 | A |
| ATPASE 6 | 8574 | C | T | 1 | H |
| ATPASE 6 | 8604 | T | C | 1 | L2 |
| ATPASE 6 | 8679 | A | G | 1 | H |
| ATPASE 6 | 8680 | C | T | 1 | D |
| ATPASE 6 | 8746 | T | G | 1 | A |
| ATPASE 6 | 8856 | G | C | 1 | K |
| ATPASE 6 | 8987 | T | C | 1 | L2 |
| ATPASE 6 | 9017 | T | C | 1 | A |
| ATPASE 6 | 9052 | A | G | 1 | L1 |
| ATPASE 6 | 9097 | A | G | 1 | B |
| ATPASE 6 | 9098 | T | C | 1 | B |
| ATPASE 6 | 9111 | T | C | 1 | L3 |
| ATPASE 6 | 9145 | G | A | 1 | W |
| ATPASE 6 | 9192 | G | A | 1 | E |
| ATPASE 6 | 9198 | C | T | 1 | W |
| CO3 | 9254 | A | G | 1 | L3 |
| CO3 | 9336 | A | G | 1 | L1 |
| CO3 | 9557 | C | T | 1 | C |
| CO3 | 9647 | T | C | 1 | L1 |
| CO3 | 9813 | T | A | 1 | K |
| CO3 | 9836 | T | C | 1 | A |
| CO3 | 9903 | T | C | 1 | L2 |
| CO3 | 9932 | G | A | 1 | L3 |
| CO3 | 9932 | G | A | 1 | B |
| TRNA-G | 9950 | T | C | 1 | B |
| TRNA-G | 10031 | T | C | 1 | A |
| ND3 | 10335 | C | T | 1 | B |
| ND4L | 10654 | C | T | 1 | H |
| ND4 | 10834 | C | T | 1 | E |
| ND4 | 10853 | C | T | 1 | C |
| ND4 | 10920 | C | T | 1 | L2 |
| ND4 | 11239 | A | G | 1 | L3 |
| ND4 | 11854 | T | C | 1 | K |
| ND4 | 11884 | A | G | 1 | B |
| ND4 | 11935 | T | C | 1 | W |
| ND4 | 12063 | C | T | 1 | U |
| ND4 | 12064 | C | T | 1 | U |
| ND4 | 12092 | C | T | 1 | C |
| TRNA-H | 12172 | A | G | 1 | L1 |
| TRNA-H | 12172 | A | G | 1 | H |
| ND5 | 12453 | T | C | 1 | H |
| ND5 | 12454 | G | A | 1 | C |
| ND5 | 12477 | T | C | 1 | L1 |
| ND5 | 12582 | A | G | 1 | U |
| ND5 | 12723 | A | G | 1 | K |
| ND5 | 12768 | A | G | 1 | L1 |
| ND5 | 12870 | C | T | 1 | L3 |
| ND5 | 13254 | T | C | 1 | E |
| ND5 | 13263 | A | G | 1 | W |
| ND5 | 13269 | A | G | 1 | L3 |
| ND5 | 13473 | A | C | 1 | U |
| ND5 | 13494 | C | T | 1 | E |
| ND5 | 13542 | A | G | 1 | L3 |
| ND5 | 13617 | T | C | 1 | U |
| ND5 | 13629 | A | G | 1 | L2 |
| ND5 | 13707 | G | A | 1 | A |
| ND5 | 13711 | G | A | 1 | H |
| ND5 | 13749 | C | T | 1 | W |
| ND5 | 13781 | T | C | 1 | H |
| ND5 | 13924 | C | T | 1 | L2 |
| ND5 | 14053 | A | G | 1 | L1 |
| ND5 | 14088 | T | C | 1 | L1 |
| ND6 | 14173 | T | C | 1 | L2 |
| ND6 | 14251 | A | G | 1 | H |
| ND6 | 14280 | A | G | 1 | A |
| ND6 | 14388 | A | G | 1 | HAPLO- |
| | | | | | GROUP |
| ND6 | 14460 | C | G | 1 | A |
| ND6 | 14548 | A | G | 1 | H |
| TRNA-E | 14693 | A | G | 1 | D |
| CYT B | 14769 | A | G | 1 | L1 |
| CYT B | 14974 | C | G | 1 | B |
| CYT B | 15016 | C | T | 1 | L1 |
| CYT B | 15043 | G | A | 1 | C |
| CYT B | 15107 | C | T | 1 | A |
| CYT B | 15266 | A | G | 1 | D |
| CYT B | 15317 | G | A | 1 | E |
| CYT B | 15386 | C | T | 1 | A |
| CYT B | 15451 | C | T | 1 | L2 |
| CYT B | 15496 | A | G | 1 | L3 |
| CYT B | 15508 | C | T | 1 | K |
| CYT B | 15511 | T | C | 1 | U |
| CYT B | 15589 | C | A | 1 | H |
| CYT B | 15616 | C | T | 1 | B |
| CYT B | 15616 | C | T | 1 | B |
| CYT B | 15766 | A | G | 1 | HAPLO- |
| | | | | | GROUP |
| CYT B | 15784 | T | C | 1 | W |
| TRNA-T | 15946 | C | T | 1 | K |
| D-LOOP | 16086 | T | C | 1 | L2 |
| D-LOOP | 16086 | T | C | 1 | L2 |
| D-LOOP | 16093 | T | C | 1 | L3 |
| D-LOOP | 16109 | A | C | 1 | C |
| D-LOOP | 16114 | C | T | 1 | H |
| D-LOOP | 16136 | T | C | 1 | B |
| D-LOOP | 16145 | G | A | 1 | L1 |
| D-LOOP | 16147 | C | T | 1 | H |
| D-LOOP | 16153 | G | A | 1 | H |
| D-LOOP | 16163 | A | G | 1 | T |
| D-LOOP | 16166 | A | G | 1 | L3 |
| D-LOOP | 16185 | C | T | 1 | L3 |
| D-LOOP | 16185 | C | T | 1 | L3 |
| D-LOOP | 16188 | C | T | 1 | C |
| D-LOOP | 16213 | G | A | 1 | L1 |
| D-LOOP | 16235 | A | G | 1 | H |
| D-LOOP | 16242 | C | T | 1 | H |
| D-LOOP | 16245 | C | T | 1 | W |
| D-LOOP | 16249 | T | C | 1 | B |
| D-LOOP | 16259 | C | T | 1 | T |
| D-LOOP | 16274 | G | A | 1 | D |
| D-LOOP | 16274 | G | A | 1 | L1 |
| D-LOOP | 16274 | G | A | 1 | T |
| D-LOOP | 16274 | G | A | 1 | H |
| D-LOOP | 16286 | C | G | 1 | L1 |
| D-LOOP | 16295 | C | T | 1 | L2 |
| D-LOOP | 16311 | T | C | 1 | C |
| D-LOOP | 16312 | A | G | 1 | B |
| D-LOOP | 16336 | G | A | 1 | A |
| D-LOOP | 16344 | C | T | 1 | B |
| D-LOOP | 16354 | C | T | 1 | D |
| D-LOOP | 16354 | C | T | 1 | H |
| D-LOOP | 16355 | C | T | 1 | L2 |
| D-LOOP | 16357 | T | C | 1 | B |
| D-LOOP | 16357 | T | C | 1 | H |
| D-LOOP | 16468 | T | C | 1 | D |
| D-LOOP | 16468 | T | C | 1 | A |
| D-LOOP | 16483 | G | A | 1 | B |
| D-LOOP | 16512 | T | C | 1 | A |
| D-LOOP | 16554 | A | G | 1 | A |

Portions of the mtDNA sequence of SEQ ID NO:1, and portions of a sample mtDNA sequence derived from a biological source or subject as provided herein, are regarded as “corresponding” nucleic acid sequences, regions, fragments or the like, based on the convention for numbering mtDNA nucleic acid positions according to SEQ ID NO:1 (Anderson et al., Nature 290:457, 1981), wherein a sample mtDNA sequence is aligned with the mtDNA sequence of SEQ ID NO:1 such that at least 70%, preferably at least 80% and more preferably at least 90% of the nucleotides in a given sequence of at least 20 consecutive nucleotides of a sequence are identical. For example, a portion of the mtDNA sequence in a biological sample containing mtDNA from a subject suspected of having or being at risk for having AD, or, as another example, a portion of the mtDNA sequence in mtDNA containing at least one mitochondrial single nucleotide polymorphism that is associated with Alzheimer's disease as provided herein (e.g., mutated mtDNA), may be aligned with a corresponding portion of the mtDNA sequence of SEQ ID NO:1 using any of a number of alignment procedures and/or tools with which those having ordinary skill in the art will be familiar (e.g., CLUSTAL W, Thompson et al., 1994 Nucl. Ac. Res. 22:4673; CAP, www.no.embnet.org/clustalw.html: FASTA/FASTP, Pearson, 1990 Proc. Nat. Acad. Sci. USA 85:2444, available from D. Hudson, Univ. of Virginia, Charlottesville, Va.). In certain preferred embodiments, a sample mtDNA sequence is greater than 95% identical to a corresponding mtDNA sequence of SEQ ID NO:1. In certain other preferred embodiments, a sample mtDNA sequence is identical to a corresponding mtDNA sequence of SEQ ID NO:1. Those oligonucleotide probes having sequences that are identical in corresponding regions of the mtDNA sequence of SEQ ID NO:1 and sample mtDNA may be identified and selected following hybridization target DNA sequence analysis, to verify the absence of mutations.

According to the present invention and as known in the art, the term “haplotype” refers to a particular combination of genetic markers in a defined region of the mitochondrial genome. Such genetic markers include, for example, RFLPs and SNPs. RFLPs (restriction fragment polymorphisms) result from an alteration in a recognition site, often a palindrome, that is specifically cleaved in a site-specific manner by a DNAse known as a restriction enzyme. A SNP (single nucleotide polymorphism) is a change (e.g., a deletion, insertion or substitution) in any single nucleotide base in a region of a genome of interest. In particularly preferred embodiments provided by the instant disclosure, the genome of interest is the mitochondrial genome. Because SNPs vary from individual to individual, they are useful markers for studying the association of a genome. Moreover, because they occur more frequently than other markers such as RFLPs, analysis of SNPs should produce a “higher resolution” picture of disease, haplogroup and individual-associated genetic marker segregation (Weiss, (1998) Genome Res. 8:691-697; Gelbert and Gregg, (1997) Curr. Opin. Biotechnol. 8:669-674).

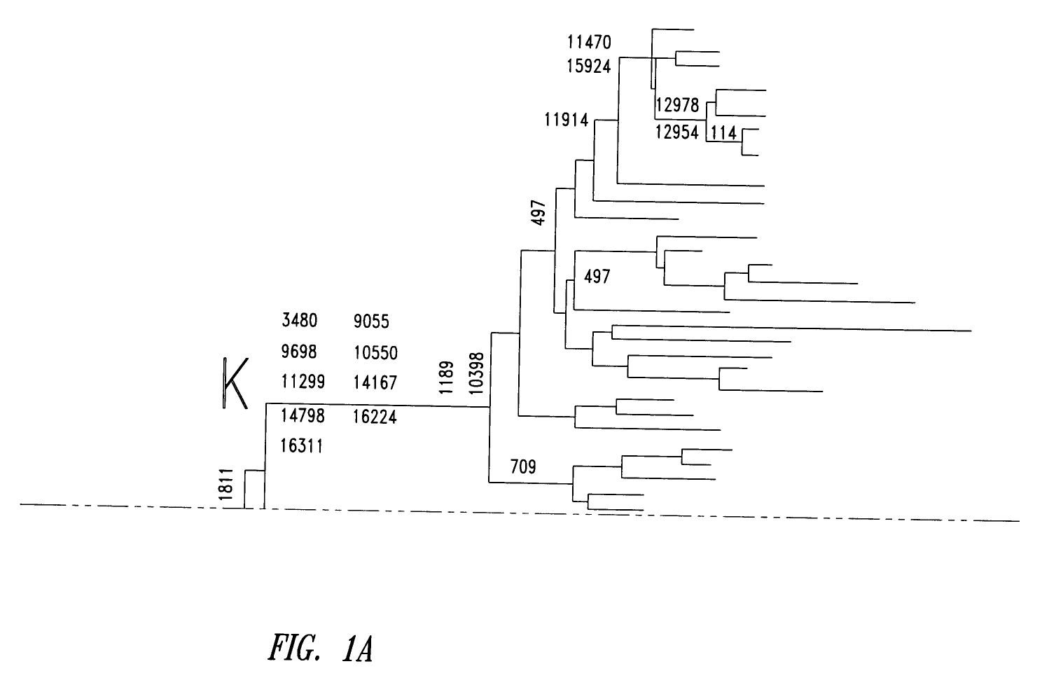

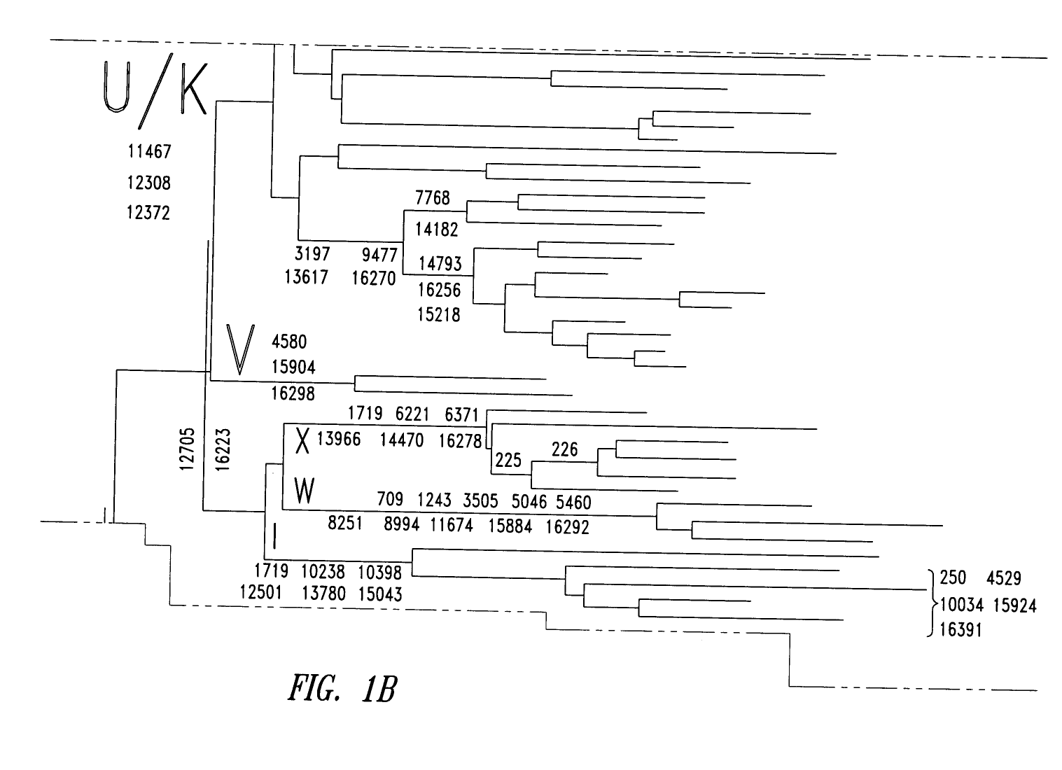

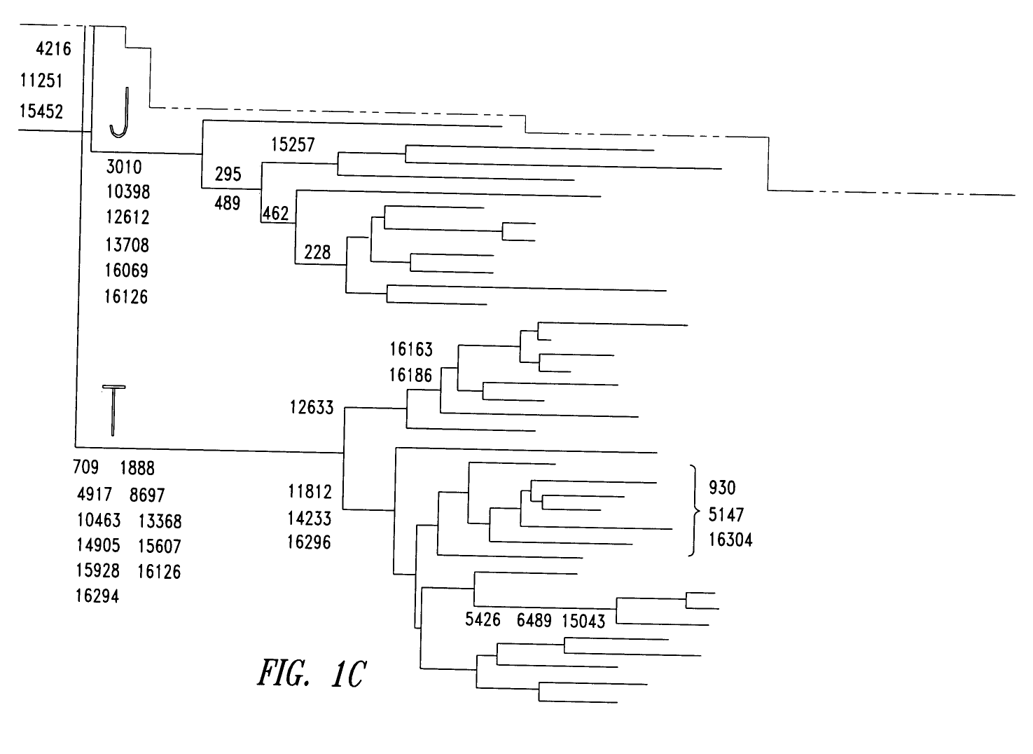

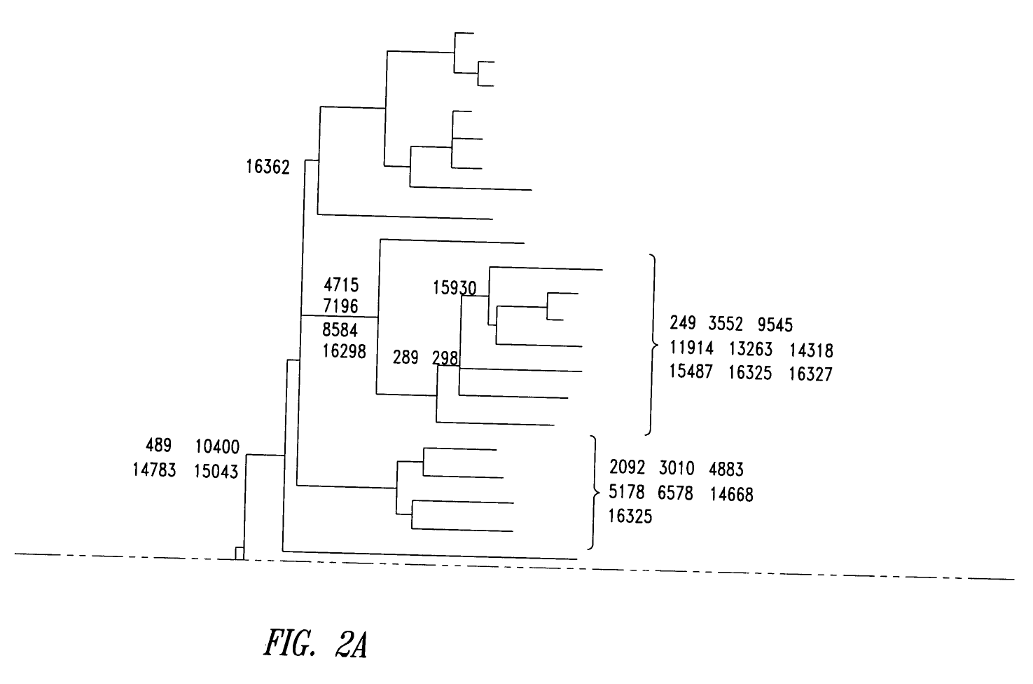

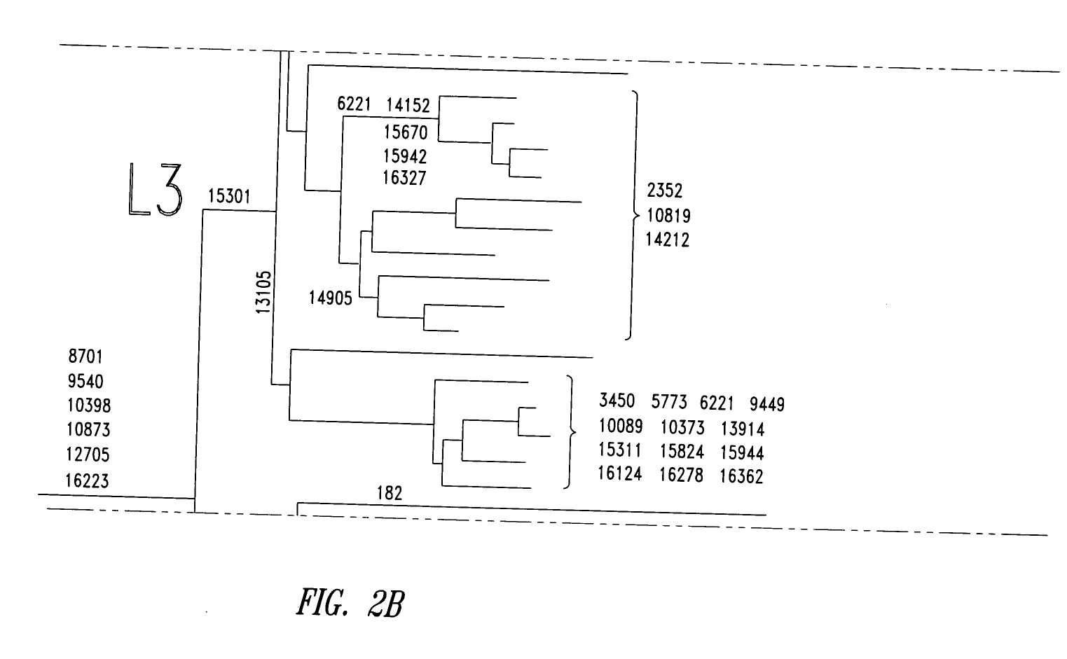

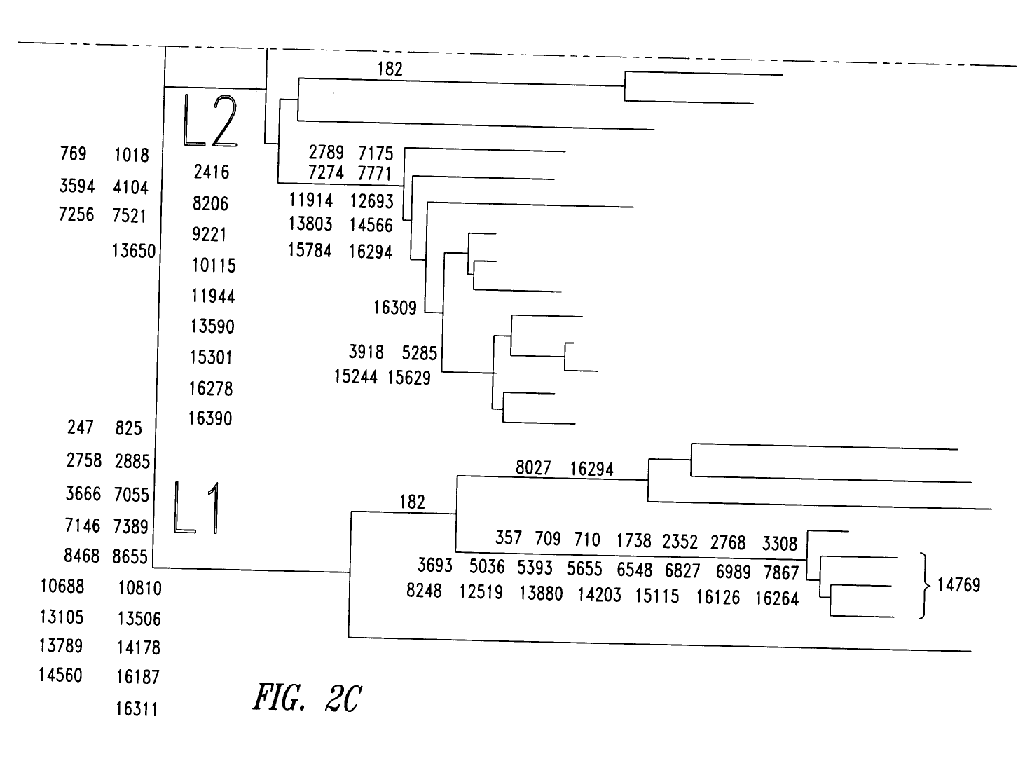

The term “haplogroup” refers to a group of haplotypes found in association with one another. Several mitochondrial DNA haplotypes and haplogroups are known in the art, including nine European mtDNA haplogroups as well as discrete Asian, Native American and African mtDNA haplogroups, each identified on the basis of the presence or absence of one or more specific restriction endonuclease recognition sites (see, e.g., Wallace et al., 1999 Gene 238:211; Torroni et al., 1996 Genetics 144:1835). In addition to haplogroups that may be regarded as clusters of haplotypes, designation of individuals as belonging to various nodes or branches within such a cluster, for example, subgroupings, subclusters, subcategories or the like, may be referred to as assignment to a “haplogroup subgroup”, as described, for example, by Macaulay et al. (1999 Am J. Hum. Genet. 64:232-249). As shown in FIGS. 1 and 2, for example, and as provided by the unexpected discovery of the present invention, particular mitochondrial haplogroups (e.g., U, K, J, T, etc.) may be divided and further subdivided into haplogroup subgroups on the basis of polymorphisms detected at nucleotide positions having the indicated numbers corresponding to nucleotide positions in SEQ ID NO:1.

Nucleic acid sequences within the scope of the invention include isolated DNA and RNA sequences that specifically hybridize under conditions of moderate or high stringency to mtDNA nucleotide sequences, including mtDNA sequences disclosed herein or fragments thereof, and their complements. As used herein, conditions of moderate stringency, as known to those having ordinary skill in the art, and as defined by Sambrook et al., Molecular Cloning: A Laboratory Manual, 2nd Ed. Vol. 1, pp. 1.101-104, Cold Spring Harbor Laboratory Press (1989), include, for example, the use as a prewashing solution for nitrocellulose filters on which proband nucleic acids have been immobilized of 5× SSC, 0.5% SDS, 1.0 mM EDTA (pH 8.0), hybridization conditions of 50% formamide, 6× SSC at 42° C. (or other similar hybridization solution), and washing conditions of about 50-60° C., 0.5× SSC, 0.1% SDS. Conditions of high stringency are defined as hybridization conditions as above, and with washing at 60-68° C., 0.2×SSC, 0.1% SDS. In other embodiments, hybridization to an mtDNA nucleotide sequence may be at normal stringency, which is approximately 25-30° C. below Tm of the native duplex (e.g., 5× SSPE, 0.5% SDS, 5× Denhardt's solution, 50% formamide, at 42° C. or equivalent conditions), at low stringency hybridizations, which utilize conditions approximately 40° C. below Tm, or at high stringency hybridizations, which utilize conditions approximately 110° C. below Tm. The skilled artisan will recognize that the temperature, salt concentration, and/or chaotrope composition of hybridization and wash solutions may be adjusted as necessary according to factors such as the length and nucleotide base composition of the probe. (See also, e.g., Ausubel et al., (1987) Current Protocols in Molecular Biology, Greene Publishing) Thus, desired variations in stringency of hybridization conditions may be achieved by altering the time, temperature and/or concentration of the solutions used for pre-hybridization, hybridization and wash steps. Accordingly, it will be appreciated that suitably stringent conditions can be readily selected without undue experimentation where a desired selectivity of the probe is identified, based on its ability to hybridize to one or more certain proband sequences while not hybridizing to certain other proband sequences.

An “isolated nucleic acid molecule” refers to a polynucleotide molecule in the form of a separate fragment or as a component of a larger nucleic acid construct, that has been separated from its source cell (including the chromosome it normally resides in) at least once, preferably in a substantially pure form. Isolated nucleic acids may be nucleic acids having particular disclosed nucleotide sequences or may be regions, portions or fragments thereof. Those having ordinary skill in the art are able to prepare isolated nucleic acids having the complete nucleotide sequence, or the sequence of any portion of a particular isolated nucleic acid molecule, when provided with the appropriate nucleic acid sequence information as disclosed herein. Nucleic acid molecules may be comprised of a wide variety of nucleotides, including DNA, RNA, nucleotide analogues such as phosphorothioates or peptide nucleic acids, or other analogues with which those skilled in the art will be familiar, or some combination of these.

The present invention, as described herein, provides mtDNA sequences and isolated mtDNA nucleic acid molecules. mtDNA may be isolated from cellular DNA according to well known methodologies, for example those described in U.S. Pat. No. 5,840,493, which is hereby incorporated by reference in its entirety. The Sequence Listing includes full-length mtDNA sequences from 560 different human subjects, as set forth at SEQ ID NOS:2-561.

Where it is advantageous to use oligonucleotide primers according to the present invention, such primers may be 10-60 nucleotides in length, preferably 15-35 nucleotides and still more preferably 18-30 nucleotides in length. Primers may be useful in the present invention for quantifying mtDNA mutations, including single nucleotide polymorphisms or homoplasmic mtDNA mutations provided herein, by any of a variety of techniques well known in the art for determining the amount of specific nucleic acid target sequences present in a sample based on specific hybridization of a primer to the target sequence. Optionally, in certain of these techniques, hybridization precedes nucleotide polymerase catalyzed extension of the primer using the strand containing the target sequence as a template, and/or ligation of oligonucleotides hybridized to adjacent target sequences, and embodiments of the invention using primer extension are particularly preferred.

For examples of references on such quantitative detection techniques, including those that may be used to detect nucleotide insertions, substitutions or deletions in a portion of an mtDNA sequence site near an oligonucleotide primer target hybridization site that corresponds to a portion of the wildtype mtDNA sequence as disclosed in Anderson et al. (1981 Nature 290:457, SEQ ID NO:1) or a mutated site such as may be created by any of the mtDNA point mutations disclosed herein, and further including those that involve primer extension, see U.S. Pat. No. 5,760,205 and the references cited therein, all of which are hereby incorporated by reference, and see also, for example, Botstein et al. (Am. J. Hum. Gen. 32:314, 1980), Gibbs et al. (Nucl. Ac. Res. 17:2437, 1989), Newton et al. (Nucl. Ac. Res. 17:2503, 1989), Grossman et al. (Nucl. Ac. Res. 22:4527, 1994), and Saiki et al. (Proc. Nat. Acad. Sci. 86:6230, 1989), all of which are hereby incorporated by reference. A particularly useful method for this purpose is the primer extension assay disclosed by Fahy et al. (Nucl. Acids Res. 25:3102, 1997) and by Ghosh et al. (Am. J. Hum. Genet. 58:325, 1996), both of which references are hereby incorporated in their entireties, as is Krook et al. (Hum. Molec. Genet. 1:391, 1995) which teaches modification of primer extension reactions to detect multiple nucleotide substitutions, insertions, deletions or other mutations. Other examples of useful techniques for quantifying the presence of specific nucleic acid target sequences in a sample include but need not be limited to labeled probe hybridization to the target nucleic acid sequences with or without first partially separating target nucleic acids from other nucleic acids present in the sample.

Examples of other useful techniques for determining the amount of specific nucleic acid target sequences present in a sample based on specific hybridization of a primer to the target sequence include specific amplification of target nucleic acid sequences and quantification of amplification products, including but not limited to polymerase chain reaction (PCR; Gibbs et al., (1989) Nucl. Ac. Res. 17:2437), transcriptional amplification systems, strand displacement amplification and self-sustained sequence replication (3SR; Ghosh et al, (1995) in Molecular Methods for Virus Detection, Academic Press, NY, pp. 287-314), the cited references for which are hereby incorporated in their entireties. Examples of other useful techniques include ligase chain reaction, single stranded conformational polymorphism analysis, Q-beta replicase assay, restriction fragment length polymorphism (RFLP; Botstein et al., (1980) Am. J. Hum. Gen. 32:314) analysis and cycled probe technology, as well as other suitable methods that will be known to those familiar with the art.

In a particularly preferred embodiment of the invention, primer extension is used to quantify mtDNA mutations present in a biological sample. (Ghosh et al., (1996) Am. J. Hum. Genet. 58:325) This embodiment may offer certain advantages by permitting both wildtype and mutant mtDNA to be simultaneously quantified using a single oligonucleotide primer capable of hybridizing to a complementary nucleic acid target sequence that is present in a defined region of wildtype mtDNA and in a corresponding region of a mutated mtDNA sequence. Without wishing to be bound by theory, the use of a single primer for quantification of wildtype and mutated mtDNA is believed to avoid uncertainties associated with potential disparities in the relative hybridization properties of multiple primers and may offer other advantages. Where such a target sequence is situated adjacent to a mutated mtDNA nucleotide sequence position that is a nucleotide substitution, insertion or deletion relative to the corresponding wildtype mtDNA sequence position, primer extension assays may be designed such that oligonucleotide extension products of primers hybridizing to mutated mtDNA are of different lengths than oligonucleotide extension products of primers hybridizing to wildtype mtDNA. Accordingly, the amount of mutant mtDNA in a sample and the amount of wildtype mtDNA in the sample may be determined by quantification of distinct extension products that are separable on the basis of sequence length or molecular mass.

Sequence length or molecular mass of primer extension assay products may be determined using any known method for characterizing the size of nucleic acid sequences with which those skilled in the art are familiar. In a preferred embodiment, primer extension products are characterized by gel electrophoresis. In another preferred embodiment, primer extension products are characterized by mass spectrometry (MS), which may further include matrix assisted laser desorption ionization/time of flight (MALDI-TOF) analysis or other MS techniques known to those having skill in the art. See, for example, U.S. Pat. No. 5,622,824, U.S. Pat. No. 5,605,798 and U.S. Pat. No. 5,547,835, all of which are hereby incorporated by reference in their entireties. In another preferred embodiment, primer extension products are characterized by liquid or gas chromatography, which may further include high performance liquid chromatography (HPLC), gas chromatography-mass spectrometry (GC-MS) or other well known chromatographic methodologies.

In another particularly preferred embodiment of the invention, DNA in a biological sample containing mtDNA is first amplified by methodologies well known in the art, such that the amplification products may be used as templates in a method for detecting single nucleotide polymorphisms or homoplasmic mtDNA mutations present in the sample. Accordingly, it may be desirable to employ oligonucleotide primers that are complementary to target sequences that are identical in, and common to, wildtype and mutant mtDNA, for example PCR amplification templates and primers prepared according to Fahy et al. (Nucl. Acids Res., 25:3102, 1997) and Davis et al. (Proc. Nat. Acad. Sci. USA 94:4526, 1997; see also Hirano et al., Proc. Nat. Acad. Sci. USA 94:14894, 1997, and Wallace et al., Proc. Nat. Acad. Sci. USA 94:14900,1997.)

In certain other preferred embodiments, mtDNA mutations may be efficiently detected, screened and/or quantified by high throughput hybridization methodologies directed to independently probing a plurality of distinct mtDNAs, or a plurality of distinct oligonucleotide primers as provided herein, that have been immobilized as nucleic acid arrays on a solid phase support. Typically, the solid support may be silica, quartz or glass, or any other material on which nucleic acid may be immobilized in a manner that permits appropriate hybridization, washing and detection steps as known in the art and as provided herein. In preferred embodiments, solid-phase nucleic acid arrays are precisely spatially addressed, as described, for example, U.S. Pat. No. 5,800,992 (see also, e.g., WO 95/21944; Schena et al., 1995 Science 270:467-470, 1995; Pease et al., 1994 Proc. Nat. Acad. Sci. USA 91:5022; Lipshutz et al., 1995 Biotechniques 19: 442-447).

Detection of hybridized (e.g., duplexed) nucleic acids on the nucleic acid array may be achieved according to any known procedure, for example, by spectrometry or potentiometry (e.g., MALDI-MS). Within certain preferred embodiments the array contains oligonucleotides that are less than 50 bp in length. For high throughput screening of nucleic acid arrays, the format is preferably amenable to automation. It is preferred, for example, that an automated apparatus for use according to high throughput screening embodiments of the present invention is under the control of a computer or other programmable controller. The controller can continuously monitor the results of each step of the nucleic acid deposition, washing, hybridization, detection and related processes, and can automatically alter the testing paradigm in response to those results.

The present invention also provides compositions and methods that are useful in pharmacogenomics, for the classification and/or stratification of a subject or a patient population. Such stratification may involve, for example, correlation of single nucleotide polymorphisms or homoplasmic mtDNA mutations as provided herein with, for instance, one or more particular traits in a subject, such as, for example, indicators of the responsiveness to, or efficacy of, a particular therapeutic treatment or characteristic genomic DNA alterations, mutations, deletions, insertions or polymorphisms.

As described herein, determination of specific single nucleotide polymorphisms or homoplasmic mtDNA mutations may be used to stratify a patient population. Accordingly, in another preferred embodiment of the invention, determination of such mutations in a biological sample from a subject diagnosed with a disease may provide a useful correlative indicator for that subject. A disease subject so classified on the basis of one or more specific mutations may then be monitored using clinical parameters referred to above and known on the art, such that correlation between particular mtDNA mutations and any particular clinical score used to evaluate a disease may be monitored. For example, stratification of an AD patient population according to at least one of the single nucleotide polymorphisms or homoplasmic mtDNA mutations provided herein may provide a useful marker with which to correlate the efficacy of any candidate therapeutic agent being used in AD subjects. In a further preferred embodiment of this aspect of the invention, determination of one or more specific mtDNA mutations in concert with determination of an AD subject's APOE genotype may also be useful. These and related advantages will be appreciated by those familiar with the art.

In particularly preferred embodiments, oligonucleotide primers will be employed that permit specific detection of the single nucleotide polymorphisms or homoplasmic mtDNA point mutations disclosed in Table 3, wherein specific substitution and deletion mutations in mitochondrial genes including, for example, those encoding 12S rRNA, 16S rRNA, several tRNAs, COX1, COX2, COX3, cytochrome b, ATPase 8, ATPase 6, ND1, ND2, ND4 and ND5 are disclosed, as are numerous mutations in the mtDNA D-loop region. Each mutation listed in Table 3 is designated with (i) the identity of the particular nucleotide position of the mutation according to the wildtype human mtDNA sequence (Anderson et al., 1981 Nature 290:457; see also Andrews et al., 1999 Nature Genetics 23:147 and references cited therein), (ii) the mitochondrial gene region according to the convention of Anderson et al. (1981) and (iii) if the mutation is not a transition (purine-to-purine or pyrimidine-to-pyrimidine), the identity of the mutated nucleotide at that position in the case of a transversion (purine-to-pyrimidine or pyrimidine-to-purine), identified as disclosed herein, or of a deletion or insertion mutation. Thus, for example, the purine nucleotide G (guanine) situated at position 3010 of the wildtype mtDNA 16S rRNA gene is mutated to the purine nucleotide A (adenosine) in mtDNA analyzed from a substantial number of haplogroup H samples (see Table 3).

TABLE 3
HAPLOGROUP-SPECIFIC AND HAPLOGROUP-ASSOCIATED
MITOCHONDRIAL SINGLE NUCLEOTIDE POLYMORPHISMS
IN MTDNA OF 560 UNRELATED INDIVIDUALS

| | | | |
|---|---|---|---|
| NP | GENE | TV/DEL/INS | HAPLOGROUP |

A. Haplogroup-specific polymorphism

| | | | |
|---|---|---|---|
| 64 | D-LOOP | | A |
| 235 | D-LOOP | | A |
| 663 | 12S rRNA | | A |
| 1598 | 12S rRNA | | A |
| 1736 | 16S rRNA | | A |
| 3316 | ND1 | | A |
| 4248 | ND1 | | A |
| 4824 | ND2 | | A |
| 4970 | ND2 | | A |
| 6308 | COI | | A |
| 7112 | COI | | A |
| 7724 | COII | A> T | A |
| 8794 | ATPase6 | | A |
| 11314 | ND4 | | A |
| 12468 | ND5 | | A |
| 12811 | ND5 | | A |
| 13855 | ND5 | | A |
| 14364 | ND6 | | A |
| 16111 | D-LOOP | | A |
| 16290 | D-LOOP | | A |
| 16319 | D-LOOP | | A |
| 827 | 12S rRNA | | B |
| 3547 | ND1 | | B |
| 4820 | ND2 | | B |
| 4977 | ND2 | | B |
| 6023 | COI | | B |
| 6216 | COI | | B |
| 6413 | COI | | B |
| 6473 | COI | | B |
| 6755 | COI | | B |
| 7241 | COI | | B |
| 9950 | COIII | | B |
| 11177 | ND4 | | B |
| 15535 | CYT B | | B |
| 16217 | D-LOOP | | B |
| 249 | D-LOOP | DEL | C |
| 289 | D-LOOP | DEL | C |
| 290 | D-LOOP | DEL | C |
| 3552 | ND1 | | C |
| 4715 | ND2 | | C |
| 7196 | COI | C> A | C |
| 7694 | COIII | | C |
| 8584 | ATPase6 | | C |
| 9545 | COIII | | C |
| 10454 | tRNA-R | | C |
| 12642 | ND5 | | C |
| 12978 | ND5 | | C |
| 14318 | ND6 | | C |
| 15487 | CYT B | A> T | C |
| 15930 | tRNA-T | | C |
| 2092 | 16S rRNA | | D |
| 4883 | ND2 | | D |
| 5178 | ND2 | C> A | D |
| 8414 | ATPase8 | | D |
| 11593 | ND4 | A> T | D |
| 14668 | ND6 | | D |
| 3027 | 16S rRNA | | E |
| 3705 | ND1 | | E |
| 4491 | ND2 | | E |
| 7598 | COII | | E |
| 239 | D-LOOP | | H |
| 456 | D-LOOP | | H |
| 477 | D-LOOP | | H |
| 951 | 12S rRNA | | H |
| 961 | 12S rRNA | | H |
| 3277 | tRNA-L | | H |
| 3333 | ND1 | | H |
| 3591 | ND1 | | H |
| 3796 | ND1 | | H |
| 3915 | ND1 | | H |
| 3992 | ND1 | | H |
| 4024 | ND1 | | H |
| 4310 | tRNA-I | | H |
| 4336 | tRNA-Q | | H |
| 4531 | ND2 | | H |
| 4727 | ND2 | | H |
| 4745 | ND2 | | H |
| 4772 | ND2 | | H |
| 4793 | ND2 | | H |
| 5004 | ND2 | | H |
| 6365 | COI | | H |
| 6776 | COI | | H |
| 6869 | COI | | H |
| 7013 | COI | | H |
| 8269 | COII | | H |
| 8448 | ATPase8 | | H |
| 8602 | ATPase6 | | H |
| 8803 | ATPase6 | | H |
| 8839 | ATPase6 | | H |
| 8843 | ATPase6 | | H |
| 8898 | ATPase6 | | H |
| 9123 | ATPase6 | | H |
| 9150 | ATPase6 | | H |
| 9380 | COIII | | H |
| 9804 | COIII | | H |
| 10044 | tRNA-G | | H |
| 11353 | ND4 | | H |
| 11560 | ND4 | | H |
| 12579 | ND5 | | H |
| 13404 | ND5 | | H |
| 13680 | ND5 | | H |
| 13759 | ND5 | | H |
| 14125 | ND5 | | H |
| 14350 | ND6 | | H |
| 14365 | ND6 | | H |
| 14470 | ND6 | T> A | H |
| 14582 | ND6 | | H |
| 14872 | CYT B | | H |
| 15466 | CYT B | | H |
| 15789 | CYT B | | H |
| 15808 | CYT B | | H |
| 15833 | CYT B | | H |
| 16162 | D-LOOP | | H |
| 16293 | D-LOOP | | H |
| 199 | D-LOOP | | I |
| 250 | D-LOOP | | I |
| 3447 | ND1 | | I |
| 3990 | ND1 | | I |
| 4529 | ND2 | A> T | I |
| 6734 | COI | | I |
| 8616 | ATPase6 | G> T | I |
| 9947 | COIII | | I |
| 10034 | tRNA-G | | I |
| 10238 | ND3 | | I |
| 11065 | ND4 | | I |
| 12501 | ND5 | | I |
| 13780 | ND5 | | I |
| 15758 | CYT B | | I |
| 16391 | D-LOOP | | I |
| 228 | D-LOOP | | J |
| 295 | D-LOOP | | J |
| 462 | D-LOOP | | J |
| 2158 | 16S rRNA | | J |
| 2387 | 16S rRNA | | J |
| 3394 | ND1 | | J |
| 5198 | ND2 | | J |
| 5633 | tRNA-A | | J |
| 6464 | COI | C> A | J |
| 6554 | COI | | J |
| 6671 | COI | | J |
| 7476 | tRNA-S | | J |
| 7711 | COII | | J |
| 10084 | ND3 | | J |
| 10172 | ND3 | | J |
| 10192 | ND3 | | J |
| 10499 | ND4L | | J |
| 10598 | ND4L | | J |
| 10685 | ND4L | | J |
| 11377 | ND4 | | J |
| 12127 | ND4 | | J |
| 12570 | ND5 | | J |
| 12612 | ND5 | | J |
| 13281 | ND5 | | J |
| 13681 | ND5 | | J |
| 13879 | ND5 | | J |
| 13933 | ND5 | | J |
| 14569 | ND6 | | J |
| 15679 | CYT B | | J |
| 15812 | CYT B | | J |
| 16069 | D-LOOP | | J |
| 16092 | D-LOOP | | J |
| 16261 | D-LOOP | | J |
| 114 | D-LOOP | | K |
| 497 | D-LOOP | | K |
| 593 | tRNA-F | | K |
| 1189 | 12S rRNA | | K |
| 2217 | 16S rRNA | | K |
| 2483 | 16S rRNA | | K |
| 3480 | ND1 | | K |
| 4295 | tRNA-I | | K |
| 4561 | ND2 | | K |
| 5814 | tRNA-C | | K |
| 6260 | COI | | K |
| 9006 | ATPase6 | | K |
| 9055 | ATPase6 | | K |
| 9698 | COIII | | K |
| 9716 | COIII | | K |
| 9962 | COIII | | K |
| 10289 | ND3 | | K |
| 10550 | ND4L | | K |
| 10978 | ND4 | | K |
| 11299 | ND4 | | K |
| 11470 | ND4 | | K |
| 11485 | ND4 | | K |
| 11840 | ND4 | | K |
| 11869 | ND4 | C> A | K |
| 11923 | ND4 | | K |
| 12954 | ND5 | | K |
| 13135 | ND5 | | K |
| 13740 | ND5 | | K |
| 13967 | ND5 | | K |
| 14002 | ND5 | | K |
| 14037 | ND5 | | K |
| 14040 | ND5 | | K |
| 14167 | ND6 | | K |
| 15884 | CYT B | | K |
| 15946 | tRNA-T | | K |
| 16224 | D-LOOP | | K |
| 16234 | D-LOOP | | K |
| 16463 | D-LOOP | | K |
| 185 | D-LOOP | G> T | L1 |
| 186 | D-LOOP | | L1 |
| 189 | D-LOOP | | L1 |
| 236 | D-LOOP | | L1 |
| 247 | D-LOOP | | L1 |
| 297 | D-LOOP | | L1 |
| 357 | D-LOOP | | L1 |
| 710 | 12S rRNA | | L1 |
| 825 | 12S rRNA | | L1 |
| 1048 | 12S rRNA | | L1 |
| 1738 | 16S rRNA | | L1 |
| 2245 | 16S rRNA | | L1 |
| 2395 | 16S rRNA | DEL | L1 |
| 2758 | 16S rRNA | | L1 |
| 2768 | 16S rRNA | | L1 |
| 2885 | 16S rRNA | | L1 |
| 3308 | ND1 | | L1 |
| 3516 | ND1 | C> A | L1 |
| 3666 | ND1 | | L1 |
| 3693 | ND1 | | L1 |
| 3777 | ND1 | | L1 |
| 3796 | ND1 | A> T | L1 |
| 3843 | ND1 | | L1 |
| 4312 | tRNA-I | | L1 |
| 4586 | ND2 | | L1 |
| 5036 | ND2 | | L1 |
| 5393 | ND2 | | L1 |
| 5442 | ND2 | | L1 |
| 5603 | tRNA-A | | L1 |
| 5655 | tRNA-A | | L1 |
| 5913 | COI | | L1 |
| 5951 | COI | | L1 |
| 6071 | COI | | L1 |
| 6150 | COI | | L1 |
| 6185 | COI | | L1 |
| 6253 | COI | | L1 |
| 6548 | COI | | L1 |
| 6827 | COI | | L1 |
| 6989 | COI | | L1 |
| 7055 | COI | | L1 |
| 7076 | COI | | L1 |
| 7146 | COI | | L1 |
| 7337 | COI | | L1 |
| 7389 | COI | | L1 |
| 7867 | COII | | L1 |
| 8248 | COII | | L1 |
| 8428 | ATPase8 | | L1 |
| 8655 | ATPase6 | | L1 |
| 8784 | ATPase6 | | L1 |
| 8877 | ATPase6 | | L1 |
| 9042 | ATPase6 | | L1 |
| 9072 | ATPase6 | | L1 |
| 9347 | COIII | | L1 |
| 9755 | COIII | | L1 |
| 9818 | COIII | | L1 |
| 10321 | ND3 | | L1 |
| 10586 | ND4L | | L1 |
| 10589 | ND4L | | L1 |
| 10664 | ND4L | | L1 |
| 10688 | ND4L | | L1 |
| 10792 | ND4 | | L1 |
| 10793 | ND4 | | L1 |
| 10810 | ND4 | | L1 |
| 11176 | ND4 | | L1 |
| 11641 | ND4 | | L1 |
| 11654 | ND4 | | L1 |
| 11899 | ND4 | | L1 |
| 12007 | ND4 | | L1 |
| 12049 | ND4 | | L1 |
| 12519 | ND5 | | L1 |
| 12720 | ND5 | | L1 |
| 12810 | ND5 | | L1 |
| 13149 | ND5 | | L1 |
| 13276 | ND5 | | L1 |
| 13485 | ND5 | | L1 |
| 13506 | ND5 | | L1 |
| 13789 | ND5 | | L1 |
| 13880 | ND5 | C> A | L1 |
| 13980 | ND5 | | L1 |
| 14000 | ND5 | T> A | L1 |
| 14148 | ND5 | | L1 |
| 14178 | ND6 | | L1 |
| 14203 | ND6 | | L1 |
| 14308 | ND6 | | L1 |
| 14560 | ND6 | | L1 |
| 14769 | CYT B | | L1 |
| 14911 | CYT B | | L1 |
| 15115 | CYT B | | L1 |
| 15136 | CYT B | | L1 |
| 16148 | D-LOOP | | L1 |
| 16187 | D-LOOP | | L1 |
| 16188 | D-LOOP | C> G | L1 |
| 16230 | D-LOOP | | L1 |
| 16264 | D-LOOP | | L1 |
| 16265 | D-LOOP | | L1 |
| 16360 | D-LOOP | | L1 |
| 16527 | D-LOOP | | L1 |
| 143 | D-LOOP | | L2 |
| 1442 | 12S rRNA | | L2 |
| 1706 | 16S rRNA | | L2 |
| 2332 | 16S rRNA | | L2 |
| 2358 | 16S rRNA | | L2 |
| 2416 | 16S rRNA | | L2 |
| 2789 | 16S rRNA | | L2 |
| 3495 | ND1 | C> A | L2 |
| 3918 | ND1 | | L2 |
| 4158 | ND1 | | L2 |
| 4185 | ND1 | | L2 |
| 4370 | tRNA-Q | | L2 |
| 4767 | ND2 | | L2 |
| 5027 | ND2 | | L2 |
| 5285 | ND2 | | L2 |
| 5331 | ND2 | C> A | L2 |
| 5581 | tRNA-W | | L2 |
| 5744 | NON-CODING | | L2 |
| 6713 | COI | | L2 |
| 7175 | COI | | L2 |
| 7274 | COI | | L2 |
| 7624 | COII | T> A | L2 |
| 7771 | COII | | L2 |
| 8080 | COII | | L2 |
| 8206 | COII | | L2 |
| 8387 | ATPase8 | | L2 |
| 8541 | ATPase8 | | L2 |
| 8790 | ATPase6 | | L2 |
| 8925 | ATPase6 | | L2 |
| 9221 | COIII | | L2 |
| 10115 | ND3 | | L2 |
| 11944 | ND4 | | L2 |
| 12236 | tRNA-S | | L2 |
| 12630 | ND5 | | L2 |
| 12693 | ND5 | | L2 |
| 12948 | ND5 | | L2 |
| 13803 | ND5 | | L2 |
| 14059 | ND5 | | L2 |
| 14544 | ND6 | | L2 |
| 14566 | ND6 | | L2 |
| 14599 | ND6 | | L2 |
| 15110 | CYT B | | L2 |
| 15217 | CYT B | | L2 |
| 15229 | CYT B | | L2 |
| 15236 | CYT B | | L2 |
| 15244 | CYT B | | L2 |
| 15391 | CYT B | | L2 |
| 15629 | CYT B | | L2 |
| 15945 | tRNA-T | T-INS | L2 |
| 16114 | D-LOOP | C> A | L2 |
| 16213 | D-LOOP | | L2 |
| 16309 | D-LOOP | | L2 |
| 16390 | D-LOOP | | L2 |
| 200 | D-LOOP | | L3 |
| 2000 | 16S rRNA | | L3 |
| 3438 | ND1 | | L3 |
| 3450 | ND1 | | L3 |
| 5773 | tRNA-C | | L3 |
| 6524 | COI | | L3 |
| 6587 | COI | | L3 |
| 6680 | COI | | L3 |
| 7424 | COI | | L3 |
| 7618 | COII | | L3 |
| 8616 | ATPase6 | | L3 |
| 8618 | ATPase6 | | L3 |
| 8650 | ATPase6 | | L3 |
| 9554 | COIII | | L3 |
| 10086 | ND3 | | L3 |
| 10373 | ND3 | | L3 |
| 10667 | ND4L | | L3 |
| 10819 | ND4 | | L3 |
| 11800 | ND4 | | L3 |
| 13101 | ND5 | A> C | L3 |
| 13886 | ND5 | | L3 |
| 13914 | ND5 | C> A | L3 |
| 14152 | ND6 | | L3 |
| 14212 | ND6 | | L3 |
| 14284 | ND6 | | L3 |
| 15099 | CYT B | | L3 |
| 15311 | CYT B | | L3 |
| 15670 | CYT B | | L3 |
| 15824 | CYT B | | L3 |
| 15942 | tRNA-T | | L3 |
| 15944 | tRNA-T | DEL | L3 |
| 16124 | D-LOOP | | L3 |
| 16327 | D-LOOP | | L3 |
| 930 | 12S rRNA | | T |
| 2141 | 16S rRNA | | T |
| 2850 | 16S rRNA | | T |
| 4688 | ND2 | | T |
| 4917 | ND2 | | T |
| 5277 | ND2 | | T |
| 6489 | COI | C> A | T |
| 7022 | COI | | T |
| 8572 | ATPase6 | | T |
| 8697 | ATPase6 | | T |
| 9117 | ATPase6 | | T |
| 9899 | COIII | | T |
| 10463 | tRNA-R | | T |
| 11242 | ND4 | | T |
| 11812 | ND4 | | T |
| 12633 | ND5 | C> A | T |
| 13368 | ND5 | | T |
| 13758 | ND5 | C> A | T |
| 13965 | ND5 | | T |
| 14233 | ND6 | | T |
| 14687 | tRNA-E | | T |
| 14905 | CYT B | | T |
| 15028 | CYT B | | T |
| 15274 | CYT B | | T |
| 15607 | CYT B | | T |
| 15928 | tRNA-T | | T |
| 16163 | D-LOOP | | T |
| 16182 | D-LOOP | A> C | T |
| 16186 | D-LOOP | | T |
| 16294 | D-LOOP | | T |
| 16296 | D-LOOP | | T |
| 16324 | D-LOOP | | T |
| 988 | 12S rRNA | | U |
| 1700 | 16S rRNA | | U |
| 1721 | 16S rRNA | | U |
| 2294 | 16S rRNA | | U |
| 3116 | 16S rRNA | | U |
| 3197 | 16S rRNA | | U |
| 3348 | ND1 | | U |
| 3720 | ND1 | | U |
| 3849 | ND1 | | U |
| 4553 | ND2 | | U |
| 4646 | ND2 | | U |
| 4703 | ND2 | | U |
| 4732 | ND2 | | U |
| 4811 | ND2 | | U |
| 5319 | ND2 | | U |
| 5390 | ND2 | | U |
| 5495 | ND2 | | U |
| 5656 | NON-CODING | | U |
| 5999 | COI | | U |
| 6045 | COI | | U |
| 6047 | COI | | U |
| 6146 | COI | | U |
| 6518 | COI | | U |
| 6629 | COI | | U |
| 6719 | COI | | U |
| 7109 | COI | | U |
| 7385 | COI | | U |
| 7768 | COII | | U |
| 7805 | COII | | U |
| 8473 | ATPase8 | | U |
| 9070 | ATPase6 | T> G | U |
| 9266 | COIII | | U |
| 9477 | COIII | | U |
| 9667 | COIII | | U |
| 10506 | ND4L | | U |
| 10876 | ND4 | | U |
| 10907 | ND4 | | U |
| 10927 | ND4 | | U |
| 11197 | ND4 | | U |
| 11332 | ND4 | | U |
| 11732 | ND4 | | U |
| 12346 | ND5 | | U |
| 12557 | ND5 | | U |
| 12618 | ND5 | | U |
| 13020 | ND5 | | U |
| 13617 | ND5 | | U |
| 13637 | ND5 | | U |
| 14139 | ND5 | | U |
| 14179 | ND6 | | U |
| 14182 | ND6 | | U |
| 14620 | ND6 | | U |
| 14793 | CYT B | | U |
| 14866 | CYT B | | U |
| 15191 | CYT B | | U |
| 15218 | CYT B | | U |
| 15454 | CYT B | | U |
| 15693 | CYT B | | U |
| 15907 | tRNA-T | | U |
| 16051 | D-LOOP | | U |
| 16256 | D-LOOP | | U |
| 16270 | D-LOOP | | U |
| 16399 | D-LOOP | | U |
| 4580 | ND2 | | V |
| 15904 | tRNA-T | | V |
| 194 | D-LOOP | | W |
| 1243 | 12S rRNA | | W |
| 1406 | 12S rRNA | | W |
| 3505 | ND1 | | W |
| 6528 | COI | | W |
| 8994 | ATPase6 | | W |
| 10097 | ND3 | | W |
| 11674 | ND4 | | W |
| 11947 | ND4 | | W |
| 12414 | ND5 | | W |
| 13263 | ND5 | | W |
| 15775 | CYT B | | W |
| 15884 | CYT B | G> C | W |
| 16292 | D-LOOP | | W |
| 225 | D-LOOP | | X |
| 226 | D-LOOP | | X |
| 6371 | COI | | X |
| 8393 | ATPase8 | | X |
| 8705 | ATPase6 | | X |
| 14470 | ND6 | | X |
| 15927 | tRNA-T | | X |
| −73 | D-LOOP | | not present |
| | | | in [H] |
| −7028 | COI | | not present |
| | | | in [H] |
| ##### | ND4 | | not present |
| | | | in [H] |
| 14766 | CYT B | | not present |
| | | | in [H] |

B. Haplogroup-associated polymorphisms

| | | | |
|---|---|---|---|
| 16362 | D-LOOP | | A, C, D, E, |
| | | | H, L3 |
| 12705 | ND5 | | A, C, D, E, |
| | | | I, L1, L2, |
| | | | L3, W, X |
| 1888 | 16S rRNA | | A, C, T |
| 13708 | ND5 | | A, J, X |
| 8027 | COII | | A, L1 |
| 153 | D-LOOP | | A, X |
| 207 | D-LOOP | | B, I, L2, W |
| 13590 | ND5 | | B, L2 |
| 9449 | COIII | | B, L3 |
| 499 | D-LOOP | | B, U |
| 16325 | D-LOOP | | C, D |
| 10400 | ND3 | | C, D, E |
| 14783 | CYT B | | C, D, E |
| 10398 | ND3 | | C, D, E, I, |
| | | | J, K, L1, |
| | | | L2, L3 |
| 15043 | CYT B | | C, D, E, |
| | | | I, T |
| 489 | D-LOOP | | C, D, E, J |
| 8701 | ATPase6 | | C, D, E, |
| | | | L1, L2, L3 |
| 9540 | COIII | | C, D, E, |
| | | | L1, L2, L3 |
| 10873 | ND4 | | C, D, E, |
| | | | L1, L2, L3 |
| 15301 | CYT B | | C, D, E, |
| | | | L2, L3 |
| 11914 | ND4 | | C, K, L1, L2 |
| 16298 | D-LOOP | | C, V |
| 3010 | 16S rRNA | | D, H, J, U |
| 16311 | D-LOOP | | H, K, L1, L3 |
| 93 | D-LOOP | | H, L1 |
| 16304 | D-LOOP | | H, T |
| 16291 | D-LOOP | | H, U |
| 16356 | D-LOOP | | H, U |
| 16482 | D-LOOP | | H, U |
| 15924 | tRNA-T | | I, K, U |
| 10915 | ND4 | | I, L1 |
| 204 | D-LOOP | | I, L2, W |
| 8251 | COII | | I, W |
| 1719 | 16S rRNA | | I, X |
| 14798 | CYT B | | J, K |
| 15257 | CYT B | | J, K |
| 5460 | ND2 | | J, L1, W |
| 185 | D-LOOP | | J, L3 |
| 11002 | ND4 | | J, L3 |
| 4216 | ND1 | | J, T |
| 11251 | ND4 | | J, T |
| 15452 | CYT B | C> A | J, T |
| 16126 | D-LOOP | | J, T |
| 9548 | COIII | | J, U |
| 13934 | ND5 | | J, U |
| 5231 | ND2 | | K, L1 |
| 709 | 12S rRNA | | K, L1, T, W |
| 1811 | 16S rRNA | | K, U |
| 11467 | ND4 | | K, U |
| 12308 | tRNA-L | | K, U |
| 12372 | ND5 | | K, U |
| 182 | D-LOOP | | L1, L2 |
| 198 | D-LOOP | | L1, L2 |
| 769 | 12S rRNA | | L1, L2 |
| 1018 | 12S rRNA | | L1, L2 |
| 3594 | ND1 | | L1, L2 |
| 4104 | ND1 | | L1, L2 |
| 7256 | COI | | L1, L2 |
| 7521 | tRNA-D | | L1, L2 |
| 13650 | ND5 | | L1, L2 |
| 16278 | D-LOOP | | L1, L2, |
| | | | L3, X |
| 189 | D-LOOP | A> C | L1, L3 |
| 2352 | 16S rRNA | | L1, L3 |
| 13105 | ND5 | | L1, L3 |
| 5046 | ND2 | | L1, W |
| 6152 | COI | | L2, U |
| 15784 | CYT B | | L2, U, W |
| 5147 | ND2 | | L3, T |
| 6221 | COI | | L3, X |
| 5426 | ND2 | | T, U |
| 13734 | ND5 | | T, U |
| 13966 | ND5 | | T, X |

TV/DEL/INS column: all nucleotide substitutions are transitions unless indicated otherwise NP: nucleotide position; TV: transversion; DEL: deletion; INS: insertion

The data of Table 3 are also depicted in Table 4, wherein the frequencies of occurrence of particular “haplogroup-specific” (e.g., characteristic of only a single haplogroup) or “haplogroup-associated” (e.g., detected in two or more identified haplogroups) mitochondrial single nucleotide polymorphism among the 560 unrelated individuals analyzed are presented; full length mtDNA sequences of these 560 individuals are set forth at SEQ ID NOS:2-561 in the Sequence Listing.

TABLE 4
HAPLOGROUP-SPECIFIC AND HAPLOGROUP-ASSOCIATED POLYMORPHISMS
IN MTDNA OF 560 UNRELATED INDIVIDUALS

| | | | HAPLOGROUP | | | | | | | | | | | | | | | | | | |
|---|---|---|---|---|---|---|---|---|---|---|---|---|---|---|---|---|---|---|---|---|---|
| | | | A | B | C | D | E | H | I | J | K | L1 | L2 | L3 | M | T | U | V | W | X | |
| | | TV/ | N | N | N | N | N | N | N | N | N | N | N | N | N | N | N | N | N | N | |
| | | DEL/ | = | = | = | = | = | = | = | = | = | = | = | = | = | = | = | = | = | = | |
| NP | GENE | INS | 25 | 18 | 13 | 9 | 3 | 226 | 14 | 33 | 47 | 13 | 23 | 20 | 1 | 46 | 42 | 8 | 8 | 11 | REF |
| 64 | D-LOOP | | 20 | 1 | | 1 | | 4 | | | | | | | | | | | | | |
| −73 | D-LOOP | | 25 | 18 | 13 | 9 | 3 | 19 | 12 | 23 | 44 | 10 | 23 | 20 | 1 | 38 | 35 | | 7 | 10 | |
| 93 | D-LOOP | | | | | | | 15 | | | | 3 | | | | | | | | | |
| 114 | D-LOOP | | | | | | | | | | 8 | | | | | | | | | | |
| 143 | D-LOOP | | | | | | | | | | | | 4 | | | | | | | | |
| 153 | D-LOOP | | 19 | | | | 1 | 1 | | | | | | | | | | | | 7 | |
| 182 | D-LOOP | | | | | | | | | | | 9 | 4 | | | | | | | | |
| 185 | D-LOOP | | | | | | 1 | | | 12 | | 2 | 1 | 5 | | | | | | | |
| 185 | D-LOOP | G > T | | | | | | | | 1 | | 6 | | | | | | | | | |
| 186 | D-LOOP | | | | | | | | | | | 4 | | | | | | | | | |
| 189 | D-LOOP | | | | | | | | | | | 4 | | | | | | | | | |
| 189 | D-LOOP | A > C | 1 | | | | 1 | | | | 1 | 4 | | 7 | | | | | 7 | | |
| 194 | D-LOOP | | | | | | | | | | | | | | | | | | 5 | 1 | |
| 198 | D-LOOP | | | | | | | | | | | 3 | 3 | | | | | | | | |
| 199 | D-LOOP | | | | | | | 2 | 11 | | 2 | | | | 1 | | | | | | |
| 200 | D-LOOP | | | | | | | 1 | | | | | | 7 | | | | | | 1 | |
| 204 | D-LOOP | | | | | | | 3 | 9 | | | 2 | 3 | | | | | | 7 | | |
| 207 | D-LOOP | | | 3 | | | | 1 | 7 | | | 1 | 2 | | | | | | 6 | | |
| 225 | D-LOOP | | | | | | | | | | | | | | | | | | | 7 | |
| 226 | D-LOOP | | | | | | | 1 | | | | | | | | | | | | 5 | |
| 228 | D-LOOP | | | 1 | | | | | | 13 | | | | | | | | | | | |
| 235 | D-LOOP | | 24 | | | | | | | 1 | | | | | | | | | | | |
| 236 | D-LOOP | | | | | | | 1 | | | | 3 | | | | | | | | | |
| 239 | D-LOOP | | | | | | | 10 | | | | | | | | | | | | | |
| 247 | D-LOOP | | | | | | | | | | | 12 | | | | | | | | | |
| 249 | D-LOOP | DEL | | | 12 | | | | | | | | | | | | | | | | |
| 250 | D-LOOP | | | | | | | | 10 | | | | | | | | | | | | |
| 289 | D-LOOP | DEL | | | 10 | | | | | | | | | | | | | | | | |
| 290 | D-LOOP | DEL | | | 10 | | | | | | | | | | | | | | | | |
| 295 | D-LOOP | | | | | | | 1 | | 23 | | | | | | | | | | | |
| 297 | D-LOOP | | | | | | | | | | | 4 | | | | | | | | | |
| 357 | D-LOOP | | | | | | | | | | | 6 | | | | | | | | | |
| 456 | D-LOOP | | | | | | | 16 | | | | | 1 | | | | | | | | |
| 462 | D-LOOP | | | | | | | | | 18 | | | | | | | | | | | |
| 477 | D-LOOP | | | | | | | 13 | | | 1 | | | | | | | | | | |
| 489 | D-LOOP | | | | 13 | 9 | 3 | | | 23 | | | | | 1 | | | | | | |
| 497 | D-LOOP | | | | | | | | | | 25 | | | | | | | | | | |
| 499 | D-LOOP | | | 17 | | | | | | | | | | | | 1 | 3 | | | | |
| 593 | tRNA-F | | | | | 1 | | | | | | | | | | | | | | | |
| 663 | 12S | | 25 | | | | | | | | | | | | | | | | | | 3-6, |
| | rRNA | | | | | | | | | | | | | | | | | | | | 14-17 |
| 709 | 12S | | | 2 | | | | 3 | | | 9 | 7 | | 1 | | 46 | 1 | | 8 | | 1 |
| | rRNA | | | | | | | | | | | | | | | | | | | | |
| 710 | 12S | | | | | | | | | | | 7 | | | | | | | | | |
| | rRNA | | | | | | | | | | | | | | | | | | | | |
| 750 | 12S | | 25 | 18 | 13 | 9 | 3 | 218 | 14 | 33 | 47 | 13 | 23 | 17 | 1 | 46 | 42 | 8 | 8 | 11 | |
| | rRNA | | | | | | | | | | | | | | | | | | | | |
| 769 | 12S | | | | | | | | | | | 13 | 23 | | | | | | | | |
| | rRNA | | | | | | | | | | | | | | | | | | | | |
| 825 | 12S | | | | | | | | | | | 13 | | | | | | | | | |
| | rRNA | | | | | | | | | | | | | | | | | | | | |
| 827 | 12S | | | 17 | | | | | | | | | | | | | | | | | |
| | rRNA | | | | | | | | | | | | | | | | | | | | |
| 930 | 12S | | 1 | | | | | | | | | | 1 | | | 16 | | | | | 1 |
| | rRNA | | | | | | | | | | | | | | | | | | | | |
| 951 | 12S | | | | | | | 3 | | | | | | | | | | | | | 1 |
| | rRNA | | | | | | | | | | | | | | | | | | | | |
| 961 | 12S | | | | | | | 4 | | | | | | | | | | | | | |
| | rRNA | | | | | | | | | | | | | | | | | | | | |
| 988 | 12S | | | | | | | | | | | | | | | | 3 | | | | |
| | rRNA | | | | | | | | | | | | | | | | | | | | |
| 1018 | 12S | | | | | | | | | | | 13 | 23 | | | | | | | | |
| | rRNA | | | | | | | | | | | | | | | | | | | | |
| 1048 | 12S | | | | | | | | | | | 2:L1a | | | | | | | | | |
| | rRNA | | | | | | | | | | | | | | | | | | | | |
| 1189 | 12S | | | | | | | | | | 34 | | | | | | | | | | 1 |
| | rRNA | | | | | | | | | | | | | | | | | | | | |
| 1243 | 12S | | | | | | | | | | | | | | | | | | 8 | | 1 |
| | rRNA | | | | | | | | | | | | | | | | | | | | |
| 1406 | 12S | | | | | | | | | | | | | | | | | | 5 | | |
| | rRNA | | | | | | | | | | | | | | | | | | | | |
| 1438 | 12S | | 25 | 18 | 13 | 9 | 3 | 208 | 12 | 33 | 47 | 6 | 23 | 20 | 1 | 46 | 42 | 8 | 8 | 11 | |
| | rRNA | | | | | | | | | | | | | | | | | | | | |
| 1442 | 12S | | | | | | | | | | | | 3 | | | | | | | | |
| | rRNA | | | | | | | | | | | | | | | | | | | | |
| 1598 | 12S | | 2 | | | | | | 1 | | | | | | | 1 | | | | | |
| | rRNA | | | | | | | | | | | | | | | | | | | | |
| 1700 | 16S | | | | | | | | | | | | | | | | 4 | | | | |
| | rRNA | | | | | | | | | | | | | | | | | | | | |
| 1706 | 16S | | | | | | | | | | | | 3 | | | | | | | | |
| | rRNA | | | | | | | | | | | | | | | | | | | | |
| 1719 | 16S | | | | | | | 2 | 14 | 1 | | | | | | 1 | | | | 11 | 1, 3, |
| | rRNA | | | | | | | | | | | | | | | | | | | | 5, 6, |
| | | | | | | | | | | | | | | | | | | | | | 9, 12, |
| | | | | | | | | | | | | | | | | | | | | | 14 |
| 1721 | 16S | | | | | | | | | | | | | | | | 4 | | | | 1 |
| | rRNA | | | | | | | | | | | | | | | | | | | | |
| 1736 | 16S | | 25 | | | | | | | | | | | | | | | | | | |
| | rRNA | | | | | | | | | | | | | | | | | | | | |
| 1738 | 16S | | | | | | | | | | | 7 | | | | | | | | | |
| | rRNA | | | | | | | | | | | | | | | | | | | | |
| 1811 | 16S | | | | | | | 1 | | | 46 | | | | | | 15 | | | | 1 |
| | rRNA | | | | | | | | | | | | | | | | | | | | |
| 1888 | 16S | | 2 | | 6 | | | | | 1 | | | | | | 46 | | | | | 1 |
| | rRNA | | | | | | | | | | | | | | | | | | | | |
| 2000 | 16S | | | | | | | | | | | | | 2 | | | | | | | 10 |
| | rRNA | | | | | | | | | | | | | | | | | | | | |
| 2092 | 16S | | | | | 9 | | | | | | | | | | | | | | | 14 |
| | rRNA | | | | | | | | | | | | | | | | | | | | |
| 2141 | 16S | | | | | | | | | | | | | | | 3 | | | | | |
| | rRNA | | | | | | | | | | | | | | | | | | | | |
| 2158 | 16S | | | | | | | | | 2 | | | | | | | 1 | | | | 1 |
| | rRNA | | | | | | | | | | | | | | | | | | | | |
| 2217 | 16S | | | | | | | 1 | | | 5 | | | | | | | | | | |
| | rRNA | | | | | | | | | | | | | | | | | | | | |
| 2245 | 16S | | | | | | | | | | | 2:L1a | | | | | | | | | |
| | rRNA | | | | | | | | | | | | | | | | | | | | |
| 2294 | 16S | | | | | | | | | | | | | | | | 3 | | | | |
| | rRNA | | | | | | | | | | | | | | | | | | | | |
| 2332 | 16S | | | | | | | | | | | | 4 | 2 | | | | | | | |
| | rRNA | | | | | | | | | | | | | | | | | | | | |
| 2352 | 16S | | | | | | | | | | | 7 | | 11 | | | | | | | 2, 3, |
| | rRNA | | | | | | | | | | | | | | | | | | | | 6, 8, |
| | | | | | | | | | | | | | | | | | | | | | 10 |
| 2358 | 16S | | | | | | | | | | | | 3 | | | | | | | | |
| | rRNA | | | | | | | | | | | | | | | | | | | | |
| 2387 | 16S | | | | | | | | | 2 | | | | | | | | | | | |
| | rRNA | | | | | | | | | | | | | | | | | | | | |
| 2395 | 16S | DEL | | | | | | | | | | 4 | | | | | | | | | |
| | rRNA | | | | | | | | | | | | | | | | | | | | |
| 2416 | 16S | | | | | | | | | | | | 23 | | | | | | | | |
| | rRNA | | | | | | | | | | | | | | | | | | | | |
| 2483 | 16S | | | | | | | | | | 2 | | | | | | | | | | 1 |
| | rRNA | | | | | | | | | | | | | | | | | | | | |
| 2706 | 16S | | 25 | 18 | 13 | 9 | 3 | 13 | 14 | 31 | 47 | 13 | 23 | 20 | 1 | 46 | 42 | 8 | 8 | 11 | |
| | rRNA | | | | | | | | | | | | | | | | | | | | |
| 2758 | 16S | | | | | | | | | | | 13 | | | | | | | | | 2, 10 |
| | rRNA | | | | | | | | | | | | | | | | | | | | |
| 2768 | 16S | | | | | | | | | | | 7 | | | | | | | | | |
| | rRNA | | | | | | | | | | | | | | | | | | | | |
| 2789 | 16S | | | | | | | | | | | | 18 | | | | | | | | |
| | rRNA | | | | | | | | | | | | | | | | | | | | |
| 2850 | 16S | | | | | | | | | | | | | | | 3 | | | | | |
| | rRNA | | | | | | | | | | | | | | | | | | | | |
| 2885 | 16S | | | | | | | | | | | 13 | | | | | | | | | |
| | rRNA | | | | | | | | | | | | | | | | | | | | |
| 3010 | 16S | | | | | 9 | | 73 | | 27 | | | 1 | 1 | | | 3 | | | | 1 |
| | rRNA | | | | | | | | | | | | | | | | | | | | |
| 3027 | 16S | | | | | | 1 | 1 | | | | | | | | | | | | | |
| | rRNA | | | | | | | | | | | | | | | | | | | | |
| 3116 | 16S | | | | | | | | | | | | | | | | 2 | | | | 1 |
| | rRNA | | | | | | | | | | | | | | | | | | | | |
| 3197 | 16S | | | | | 1 | | | | | | | | | | | 24 | | | | 1, 4 |
| | rRNA | | | | | | | | | | | | | | | | | | | | |
| 3277 | tRNA-L | | | | | | | 2 | | | | | | | | | | | | | |
| 3308 | ND1 | | | | | | | | | | | 7 | 1 | | | | | | | | |
| 3316 | ND1 | | 2 | | | | | 1 | | | | | 1 | | | 1 | | | 1 | | |
| 3333 | ND1 | | | | | | | 2 | | | | | | | | | | | | | |
| 3348 | ND1 | | | | | | | | | | | | | | | | 2 | | | | |
| 3394 | ND1 | | | | | | 1 | 1 | | 2 | | | | | | | | | | | 1 |
| 3438 | ND1 | | | | | | | 1 | | | | | 1 | 3 | | | | | | | |
| 3447 | ND1 | | | | | | | | 4 | | | | | | | | | | | | 1 |
| 3450 | ND1 | | | | | | | | | | | | | 6 | | | | | | | |
| 3480 | ND1 | | | | | | | | | | 47 | | | | | | | | | | 1 |
| 3495 | ND1 | C > A | | | | | | | | | | | 3 | | | | | | | | |
| 3505 | ND1 | | | | | | | | | | | | | | | | | | 8 | | 1 |
| 3516 | ND1 | C > A | | | | | | | | | | 2:L1a | | | | | | | | | |
| 3547 | ND1 | | | 14 | | | | 2 | | | | | | | | | | | | | |
| 3552 | ND1 | | | | 12 | | | | | | 1 | | | | | | | | | | |
| 3591 | ND1 | | 1 | | | | | 2 | | | | | | | | | | | | | |
| 3594 | ND1 | | | | | | | | | | | 13 | 23 | | | | | | | | 2, 3, |
| | | | | | | | | | | | | | | | | | | | | | 5, 6, |
| | | | | | | | | | | | | | | | | | | | | | 8, 10 |
| 3666 | ND1 | | | | | 1 | | 2 | | | | 11 | | | | | | | | | |
| 3693 | ND1 | | | | | | | | | | | 7 | 1 | | | | | | | | |
| 3705 | ND1 | | | | | | 2 | | | | | | 1 | | | | 1 | | | 1 | |
| 3720 | ND1 | | | | | | | | | | | | | | | | 7 | | | | 1 |
| 3777 | ND1 | | | | | | | | | | | 2 | | | | | | | | | |
| 3796 | ND1 | | | 1 | | | | 3 | | | | | | | | | | | | | |
| 3796 | ND1 | A > T | | | | | | 1 | | | | 2 | | | | | | | | | |
| 3843 | ND1 | | | | | | | | | | | 2 | | | | | | | | | |
| 3849 | ND1 | | | | | | | | | | | | | | | | 2 | | | | |
| 3915 | ND1 | | | 1 | | | | 9 | | | | | | 1 | | | | | | | |
| 3918 | ND1 | | | 1 | | | | | | | | | 7 | | | 1 | 1 | | | | |
| 3990 | ND1 | | | | | | | | 3 | | | | | | | | | | | | 1 |
| 3992 | ND1 | | | | | | | 9 | | | | | | | | | | | | | |
| 4024 | ND1 | | | | | | | 10 | | | | | | | | | | | | | |
| 4104 | ND1 | | | | | | | | | | | 13 | 23 | | | 1 | | | | | |
| 4158 | ND1 | | | | | | | | | | | | 3 | | | | | | | | 2 |
| 4185 | ND1 | | | | | | | 1 | | | | | 2 | | | | | | | | |
| 4216 | ND1 | | | | | | | 2 | | 33 | | | 1 | | | 46 | | | | | 1, 4 |
| 4248 | ND1 | | 25 | | | | | | | | | | | | | | | | | | |
| 4295 | tRNA-I | | | | | | | | | | 3 | | | | | | | | | | |
| 4310 | tRNA-I | | | | | | | 2 | | | | | | | | | | | | | |
| 4312 | tRNA-I | | | | | | | | | | | 2:L1a | | | | | | | | | 4, 10 |
| 4336 | tRNA-Q | | | | | | | 10 | | | | | | | | | | | | | 1, 12, |
| | | | | | | | | | | | | | | | | | | | | | 14 |
| 4370 | tRNA-Q | | | | | | | | | | | | 3 | | | | | | | | |
| 4491 | ND2 | | | | | | 2 | | | | | | | | | | | | | | |
| 4529 | ND2 | A > T | | | | | | | 12 | | | | | | | | | | | | 1, 3, |
| | | | | | | | | | | | | | | | | | | | | | 4, 9, |
| | | | | | | | | | | | | | | | | | | | | | 12, 14 |
| 4531 | ND2 | | | | | | | 2 | | | | | | | | | | | | | |
| 4553 | ND2 | | | | | | | | | | | | | | | | 4 | | | | |
| 4561 | ND2 | | | | | | | 1 | | | 8 | | | | | | 1 | | | | |
| 4580 | ND2 | | | | | | | | | | | | | | | | | 8 | | | 1, |
| | | | | | | | | | | | | | | | | | | | | | 3-5, |
| | | | | | | | | | | | | | | | | | | | | | 9 |
| 4586 | ND2 | | | | | | | | | | | 2:L1a | | | | | | | | | |
| 4646 | ND2 | | | | | | | 1 | | | | | | | | | 5:U4 | | | | 1, 4 |
| 4688 | ND2 | | | | | | | | | | | | | 1 | | 2 | | | | | |
| 4703 | ND2 | | | | | | | | | | | | | | | | 3 | | | | |
| 4715 | ND2 | | | | 13 | | | | | | | | | | | | | | | | |
| 4727 | ND2 | | | | | | | 5 | | | | | | | | | | | | | |
| 4732 | ND2 | | | | | | | | | | | | | | | | 2 | | | | 1 |
| 4745 | ND2 | | | | | | | 2 | | | | | | | | | | | | | 1 |
| 4767 | ND2 | | | | | | | 1 | | | | | 3 | | | | | | | | |
| 4769 | ND2 | | 25 | 18 | 13 | 9 | 3 | 209 | 14 | 33 | 47 | 13 | 23 | 20 | 1 | 46 | 42 | 8 | 8 | 11 | |
| 4772 | ND2 | | | | | | | 2 | | | | | | | | | | | | | |
| 4793 | ND2 | | | | | | | 5 | | | | | | | | 1 | | | | | |
| 4811 | ND2 | | | | | | | | | | | | | | | | 2 | | | | |
| 4820 | ND2 | | | 17 | | | | | | | | | | | | | 1 | | | | |
| 4824 | ND2 | | 25 | | | | | | | | | 1 | | | | | | | | | |
| 4883 | ND2 | | | | | 9 | | | | | | | | | | | | | | | |
| 4917 | ND2 | | | | | | | | 1 | | | | | | | 46 | | | | | 1, 4 |
| 4970 | ND2 | | 2 | | | | | | | | | | | | | | | | | | |
| 4977 | ND2 | | | 14 | | | | | | | | | | | | | | | | | |
| 5004 | ND2 | | | | | | | 11 | | | | | | | | | | | 1 | | |
| 5027 | ND2 | | | | | | | | | | | | 3 | | | | | | | | |
| 5036 | ND2 | | | | | | | | | | | 7 | | | | | | | | | |
| 5046 | ND2 | | | | | | | | | | | 7 | | 1 | | | | | 8 | | 1 |
| 5147 | ND2 | | | | | | | 1 | | | | 1 | | 3 | | 16 | | | | | 1 |
| 5178 | ND2 | C > A | | | | 9 | | | | | | | | | | | | | | | 2-6, |
| | | | | | | | | | | | | | | | | | | | | | 11, |
| | | | | | | | | | | | | | | | | | | | | | 13-17 |
| 5198 | ND2 | | | | | | | | | 3 | | | | | | | | | | | |
| 5231 | ND2 | | | | | | | | | | 5 | 2:L1a | | | | | | | | | |
| 5277 | ND2 | | | | | | | | | | | | | | | 5 | | | | | 1 |
| 5285 | ND2 | | | | | | | | | | | | 7 | | | | | | | | |
| 5319 | ND2 | | | | | | | 1 | | | | | | | | | 2 | | | | |
| 5331 | ND2 | C > A | | | | | | | | | | | 3 | | | | | | | | |
| 5390 | ND2 | | | | | | | | | | | | | | | | 7 | | | | 1 |
| 5393 | ND2 | | | | | | | | | | | 7 | | | | | | | | | |
| 5426 | ND2 | | | | | | | | | | | | | | | 7 | 7 | | | | 1 |
| 5442 | ND2 | | | | | | | 1 | | | | 2:L1a | | | 1 | | | | | | |
| 5460 | ND2 | | | | | 1 | | 1 | | 2 | | 2:L1a | 1 | | | | | 1 | 8 | | 1 |
| 5495 | ND2 | | | | | | | | | | | | | | | | 2 | | | | 1 |
| 5581 | tRNA-W | | | | | | | | | | | | 3 | | | | 1 | | | | |
| 5603 | tRNA-A | | | | | | | | | | | 2:L1a | | | | | | | | | |
| 5633 | tRNA-A | | | | | | | | | 3 | | | | | | | | | | | 1 |
| 5655 | tRNA-A | | | | | | | | | | | 7 | | | | | | | | | |
| 5656 | | | | | | | | | 1 | | | | | | | | 4 | | | | 1 |
| 5744 | | | | | | | | | | | | | 2 | | | | | | | | |
| 5773 | tRNA-C | | | | | | | | | | | | | 6 | | | | | | | |
| 5814 | tRNA-C | | | | | | | | | | | | 3 | | | | | | | | |
| 5913 | COI | | 1 | | | | | | | | 4 | | | | | | | | | | 1 |
| 5951 | COI | | | | | | | | | | | 4 | | | | | | | | | |
| 5999 | COI | | | | | | | | | | | | | | | 1 | 5:U4 | | | | 1 |
| 6023 | COI | | | 3 | | | | 2 | | | | | | | | 1 | 1 | | | | |
| 6045 | COI | | | | | | | | | | | | | | | | 7 | | | | 1 |
| 6047 | COI | | | | | | | | | | | | | | | | 5:U4 | | | | 1 |
| 6071 | COI | | | | | | | | | | | 4 | | | | | | | | | |
| 6146 | COI | | | | | | | | | | | | | | | | 2 | | | | |
| 6150 | COI | | | | | | | | | | | 2 | | 1 | | | | | | | |
| 6152 | COI | | | | | | | | | | | | 2 | | | 1 | 7 | | | | 1 |
| 6185 | COI | | | | | | | | | | | 2:L1a | | | | | | | | | |
| 6216 | COI | | | 3 | | | | | | | | | | | | | | | | | |
| 6221 | COI | | | | | | | | | | | | 1 | 11 | | 1 | | | | 11 | 1 |
| 6253 | COI | | | | | | | 1 | | | | 2 | | | | | | | | | |
| 6260 | COI | | 1 | | | 2 | | 1 | | | 6 | | | | | | 1 | | | | |
| 6308 | COI | | 4 | | | | | | | | | | | | | | | | | | |
| 6365 | COI | | | | | | | 7 | | | | | | | | | | | | | |
| 6371 | COI | | | | | | | | | | | | | | | | | | | 11 | 1 |
| 6413 | COI | | | 3 | | | | | | 1 | | | | | | | | | | | |
| 6464 | COI | C > A | | | | | | | | 2 | | | | | | | | | | | |
| 6473 | COI | | | 14 | | | | | | | | | | | | | | | | | |
| 6489 | COI | C > A | | | | | | | | | | | | | | 5 | | | | | |
| 6518 | COI | | | | | | | | | | | | | | | | 2 | | | | |
| 6524 | COI | | | | | | | | | | | | | 2 | | | | | | | |
| 6528 | COI | | | | | | | | | | | | | | | | | | 2 | | |
| 6548 | COI | | | | | | | | | | | 7 | | | | | | | | | |
| 6554 | COI | | | | | | | | | 2 | | | | | | | | | | | |
| 6587 | COI | | | | | | | | | | | | | 5 | | | | | | | |
| 6629 | COI | | | | | | | | | | | | | | | | 2 | | | | |
| 6671 | COI | | | | | | 1 | 1 | | 2 | | | | | | | | | | | |
| 6680 | COI | | | | | | 1 | | | | | | | 2 | | | | | | | |
| 6713 | COI | | | | | | | | | | | | 2 | | | | | | | | |
| 6719 | COI | | | | | | | 1 | | | | | | | | | 2 | | | | |
| 6734 | COI | | | | | | | | 4 | | | | | | | | | | | | 1 |
| 6755 | COI | | | 2 | | | | 1 | | | | | | | | | | | | | |
| 6776 | COI | | | | | | | 23 | | | | | | | | | | | | | 1 |
| 6827 | COI | | | | | | | 1 | | | 1 | 7 | | | | | | | | | |
| 6869 | COI | | | | | | | 4 | | | | | | | | | | | | | |
| 6989 | COI | | | | | | | | | | | 7 | | | | | | | | | |
| 7013 | COI | | | | | | | 3 | | | | | | | | | | | | | |
| 7022 | COI | | | | | | | | | | | | | | | 3 | | | | | |
| −7028 | COI | | 25 | 18 | 13 | 9 | 3 | 12 | 14 | 33 | 47 | 13 | 23 | 20 | 1 | 46 | 42 | 8 | 8 | 11 | 3, 9 |
| 7055 | COI | | | 1 | | | | | | | | 11 | | | | | | | | | 10 |
| 7076 | COI | | | | | | | | | | | 2 | | | | | | | | | |
| 7109 | COI | | | | | | | | | | | | | | | | 3 | | | | |
| 7112 | COI | | 3 | | | | | | | | | | | | | | | | | | |
| 7146 | COI | | | | | | | | | | | 13 | | | | | | | | | |
| 7175 | COI | | | | | | | | | | | | 18 | | | | | | | | |
| 7196 | COI | C > A | | | 13 | | | | | | | | | | | | | | | | |
| 7241 | COI | | | 2 | | | | | | | | | | | | | | | | | |
| 7256 | COI | | | | | | | | | | | 12 | 23 | | | | | | | | |
| 7274 | COI | | | | | | | | | | | | 18 | | | | | | | | |
| 7337 | COI | | | | | | | | | | | 2 | | | | | | | | | |
| 7385 | COI | | | | | | | | | | | | | | | | 2 | | | | 1 |
| 7389 | COI | | | | | | | | | | | 11 | | | | | | | | | |
| 7424 | COI | | | | | | | 1 | | | | | | 3 | | | | | | | |
| 7476 | tRNA-S | | | | | | | | | 6 | | | | | | | | | | | 1 |
| 7521 | tRNA-D | | | | | | | | | | | 13 | 23 | | | | | | | | |
| 7598 | COII | | | | | | 1 | | | | | | | | | | | | | | 13 |
| 7618 | COII | | | | | | | | | | | | | 2 | | | | | | | |
| 7624 | COII | T > A | | | | | | | | | | | 3 | | | | | | | | |
| 7694 | COII | | | | 2 | | | | | | | | | | | | | | | | |
| 7711 | COII | | | | | | | | | 2 | | | | | | | | | | | |
| 7724 | COII | A > T | 2 | | | | | | | | | | | | | | | | | | |
| 7768 | COII | | | | | | | | | | | | | | | | 8 | | | | 1 |
| 7771 | COII | | | | | | | | | | | | 18 | | | | | | | | |
| 7805 | COII | | | | | | | | | | | | | | | | 2 | | | | |
| 7867 | COII | | | | | | | | | | | 7 | | | | | | | | | |
| 8027 | COII | | 25 | | | | | | | | | 4 | | | | | | | | | |
| 8080 | COII | | | | | | | | | | | | 2 | | | | | | | | |
| 8206 | COII | | | | | | | | | | | | 23 | | | | | | | | |
| 8248 | COII | | | | | | | | | | | 7 | | | | | | | | | |
| 8251 | COII | | | | | | | | 13 | | | | | | | 1 | | | 8 | | 1, 3, |
| | | | | | | | | | | | | | | | | | | | | | 5, 9, |
| | | | | | | | | | | | | | | | | | | | | | 12, 14 |
| 8269 | COII | | | | | | | 8 | | 1 | | | | | | | | | | | |
| 8387 | ATPase | | | | | | | | | | | | 3 | | | | | | | | |
| | 8 | | | | | | | | | | | | | | | | | | | | |
| 8393 | ATPase | | | | | | | | | | | | | | | | | | | 5 | |
| | 8 | | | | | | | | | | | | | | | | | | | | |
| 8414 | ATPase | | | | | 9 | | | | | | | | | | | | | | | 14 |
| | 8 | | | | | | | | | | | | | | | | | | | | |
| 8428 | ATPase | | | | | | | | | | | 2:L1a | | | | | | | | | |
| | 8 | | | | | | | | | | | | | | | | | | | | |
| 8448 | ATPase | | | | | | | 3 | | | | | | | | | | | | | |
| | 8 | | | | | | | | | | | | | | | | | | | | |
| 8473 | ATPase | | | | | | | 4 | | | | | | | | | 2 | | | | |
| | 8 | | | | | | | | | | | | | | | | | | | | |
| 8541 | ATPase | | | | | | | | | | | | 2 | | | | | | | | |
| | 8 | | | | | | | | | | | | | | | | | | | | |
| 8572 | ATPase | | | | | | | 1 | | | | | | | | 2 | | | | | |
| | 6 | | | | | | | | | | | | | | | | | | | | |
| 8584 | ATPase | | | | 13 | | | | | | | | | | | | | | | | |
| | 6 | | | | | | | | | | | | | | | | | | | | |
| 8602 | ATPase | | | | | | | 3 | | | | | | | | | | | | | |
| | 6 | | | | | | | | | | | | | | | | | | | | |
| 8616 | ATPase | G > T | | | | | | | 4 | | | | | | | | | | | | 1 |
| | 6 | | | | | | | | | | | | | | | | | | | | |
| 8616 | ATPase | | | | | | | | | | | | | 2 | | | | | | 1 | 1, 2, |
| | 6 | | | | | | | | | | | | | | | | | | | | 10 |
| 8618 | ATPase | | | | | | | 2 | | | | | | 3 | | | | | | | 1, 3, |
| | 6 | | | | | | | | | | | | | | | | | | | | 6, 8, |
| | | | | | | | | | | | | | | | | | | | | | 10 |
| 8650 | ATPase | | | | | | | | | | | | | 4 | | | | | | | |
| | 6 | | | | | | | | | | | | | | | | | | | | |
| 8655 | ATPase | | | | | | | | | | | 13 | | | | | | | | | |
| | 6 | | | | | | | | | | | | | | | | | | | | |
| 8697 | ATPase | | | | | | | | 1 | | 1 | | | | | 46 | | | | | 1 |
| | 6 | | | | | | | | | | | | | | | | | | | | |
| 8701 | ATPase | | | | 13 | 9 | 3 | | | | | 13 | 23 | 20 | 1 | 1 | | | | | |
| | 6 | | | | | | | | | | | | | | | | | | | | |
| 8705 | ATPase | | | | | | | | | | | | | | | | | | | 3 | 1 |
| | 6 | | | | | | | | | | | | | | | | | | | | |
| 8784 | ATPase | | | | | | | | | | | 2 | | | | | | | | | |
| | 6 | | | | | | | | | | | | | | | | | | | | |
| 8790 | ATPase | | | | | | | | | | | | 3 | | | | | | | | |
| | 6 | | | | | | | | | | | | | | | | | | | | |
| 8794 | ATPase | | 25 | | | | | | | | | | | | | | | | | | |
| | 6 | | | | | | | | | | | | | | | | | | | | |
| 8803 | ATPase | | | | | | | 2 | | | | | | | | | | | | | |
| | 6 | | | | | | | | | | | | | | | | | | | | |
| 8839 | ATPase | | | | | | | 2 | | | | | | | | | | | | | |
| | 6 | | | | | | | | | | | | | | | | | | | | |
| 8843 | ATPase | | | | | | | 3 | | | | | | | | | | | | | 1 |
| | 6 | | | | | | | | | | | | | | | | | | | | |
| 8860 | ATPase | | 25 | 18 | 13 | 9 | 3 | 220 | 14 | 33 | 47 | 13 | 23 | 20 | 1 | 46 | 42 | 8 | 8 | 11 | |
| | 6 | | | | | | | | | | | | | | | | | | | | |
| 8877 | ATPase | | | | | | | | | | | 2 | | | | | | | | | |
| | 6 | | | | | | | | | | | | | | | | | | | | |
| 8898 | ATPase | | | | | | | 2 | | | | | | | | | | | | | |
| | 6 | | | | | | | | | | | | | | | | | | | | |
| 8925 | ATPase | | | | | | | | | | | | 2 | | | | | | | | |
| | 6 | | | | | | | | | | | | | | | | | | | | |
| 8994 | ATPase | | | | | | | | | | | | | | | | | | 8 | | 1, 3, |
| | 6 | | | | | | | | | | | | | | | | | | | | 4, 9 |
| 9006 | ATPase | | | | | | | | | | 2 | | | | | | | | | | |
| | 6 | | | | | | | | | | | | | | | | | | | | |
| 9042 | ATPase | | | | | | | | | | | 2:L1a | | | | | | | | | |
| | 6 | | | | | | | | | | | | | | | | | | | | |
| 9055 | ATPase | | | | | | | 1 | | | 47 | | | | | | | | | | 1, |
| | 6 | | | | | | | | | | | | | | | | | | | | 3-5, |
| | | | | | | | | | | | | | | | | | | | | | 9, 14 |
| 9070 | ATPase | T > G | | | | | | | | | | | | | | | 2 | | | | |
| | 6 | | | | | | | | | | | | | | | | | | | | |
| 9072 | ATPase | | | | | | | | | | | 4 | | | | | | | | | 2, 3, |
| | 6 | | | | | | | | | | | | | | | | | | | | 8, 10 |
| 9117 | ATPase | | | | | | | 1 | | | | | | | | 3 | | | | | |
| | 6 | | | | | | | | | | | | | | | | | | | | |
| 9123 | ATPase | | | | | | | 11 | | | | | | | | | | | | | |
| | 6 | | | | | | | | | | | | | | | | | | | | |
| 9150 | ATPase | | | | | | | 3 | | | | | | | | | | | | | |
| | 6 | | | | | | | | | | | | | | | | | | | | |
| 9221 | COIII | | | | | | | | | | | | 23 | | | | | | | | |
| 9266 | COIII | | | | | | | 1 | | | | | | | | 1 | 3 | | | | |
| 9347 | COIII | | | | | | | | | | | 2:L1a | | | | | | | | | |
| 9380 | COIII | | | | | | | 6 | | | | | | | | | | | | | |
| 9449 | COIII | | 1 | 2 | | | | | | | | | | 6 | | | | | | | |
| 9477 | COIII | | | | | | | | | | | | | | | | 23 | | | | 1 |
| 9540 | COIII | | | | 13 | 9 | 3 | | | | | 13 | 23 | 20 | 1 | | | | | | |
| 9545 | COIII | | 1 | | 12 | | 1 | | | | | 1 | | | | | | | | | |
| 9548 | COIII | | | | | | | | | 2 | | | | | | | 2 | | | | |
| 9554 | COIII | | | | | | | 1 | | | | 1 | 1 | 2 | | | | | | | |
| 9667 | COIII | | | | | | | | | | 1 | | | | | | 8 | | | | 1 |
| 9698 | COIII | | | | | | | | | | 47 | | | | | | | | | | 1 |
| 9716 | COIII | | | | | | | | | | 13 | | | | | | | | | | |
| 9755 | COIII | | | | | | | 1 | | | | 2:L1a | | | | | | | | | |
| 9804 | COIII | | | | | | | 5 | | | | | | | | | | | | | |
| 9818 | COIII | | | | | | | | | | | 2:L1a | | | | | | | | | |
| 9899 | COIII | | | | | | | | | | | | | | | 6 | | | | | |
| 9947 | COIII | | | | | | | | 3 | | | | | | | 1 | | | | | 1 |
| 9950 | COIII | | | 12 | | | | | | 1 | | | | | | | | | | | |
| 9962 | COIII | | | | | | | | | | 3 | | | | | | | | | | |
| 10034 | tRNA-G | | | | | | | | 12 | | | | | | | | | | | | 1, 4, |
| | | | | | | | | | | | | | | | | | | | | | 5, 9, |
| | | | | | | | | | | | | | | | | | | | | | 12, 14 |
| 10044 | tRNA-G | | | | | | | 5 | | | | | | | | | | | | | 7 |
| 10084 | ND3 | | | | | | | | | 4 | 1 | 1 | | | | | | | | | 1 |
| 10086 | ND3 | | | | | | | | | | | | | 6 | | | | | | | 1-3, |
| | | | | | | | | | | | | | | | | | | | | | 6, 8, |
| | | | | | | | | | | | | | | | | | | | | | 10 |
| 10097 | ND3 | | | | | | | | | | | | | | | | | | 2 | | |
| 10115 | ND3 | | | | | | | | | | | | 23 | | | | | | | | 1 |
| 10172 | ND3 | | | | | | | | | 3 | | | | | | | | | | | |
| 10192 | ND3 | | | | | | | | | 2 | | | | | | | | | | | |
| 10238 | ND3 | | | | | | | | 14 | | | | | | | | | | | | 1, 4 |
| 10289 | ND3 | | | | | | | | | | 3 | | | | | | | | | | |
| 10321 | ND3 | | | | | | | | | | | 4 | | | | | | | | | 10 |
| 10373 | ND3 | | | | | | | 1 | | | | | | 6 | | | | | 1 | | |
| 10398 | ND3 | | | | 13 | 9 | 3 | | 13 | 29 | 33 | 13 | 23 | 20 | 1 | | | | | | 1, 2, |
| | | | | | | | | | | | | | | | | | | | | | 5, 6, |
| | | | | | | | | | | | | | | | | | | | | | 8, 9, |
| | | | | | | | | | | | | | | | | | | | | | 11-17 |
| 10400 | ND3 | | | | 13 | 9 | 3 | | | | | | | | 1 | | | | | | 4-6, |
| | | | | | | | | | | | | | | | | | | | | | 11, |
| | | | | | | | | | | | | | | | | | | | | | 13-17 |
| 10454 | tRNA-R | | | | 2 | | | | | | | | 1 | | | | | | | | |
| 10463 | tRNA-R | | | | | | | | | | | | | | | 46 | | | | | 1, 4 |
| 10499 | ND4L | | | | | | | | | 3 | | | | | | | | | | | 1 |
| 10506 | ND4L | | | | | | | | | | | | | | | | 3 | | | | |
| 10550 | ND4L | | | | | | | | | | 47 | | | | | | | | | | |
| 10586 | ND4L | | | | | | | 1 | | | | 4 | | | | | | | | | |
| 10589 | ND4L | | | | | | | 2 | | | | 2:L1a | | | | 1 | | | | | |
| 10598 | ND4L | | | 1 | | 1 | | | | 2 | | | | | | | | | | | |
| 10664 | ND4L | | | | | | | | | | | 2:L1a | | | | | | | | | |
| 10667 | ND4L | | | | | | | | | | | | | 2 | | | | | | | |
| 10685 | ND4L | | | 1 | | | | 1 | | 3 | | | | | | | 1 | | | | |
| 10688 | ND4L | | | | | | | | | | | 13 | | | | | | | | | |
| 10792 | ND4 | | | | | | | | | | | 2 | | | | | | | | | |
| 10793 | ND4 | | | | | | | | | | | 2 | | | | | | | | | |
| 10810 | ND4 | | | | | | | 2 | | | | 13 | | | | | | | | | 1, 2, |
| | | | | | | | | | | | | | | | | | | | | | 5, 6, |
| | | | | | | | | | | | | | | | | | | | | | 8, 10 |
| 10819 | ND4 | | | | | | | | 1 | | | | | 11 | | | | | | | |
| 10873 | ND4 | | | | 13 | 9 | 3 | | | | | 13 | 23 | 20 | 1 | | | | | | 6 |
| 10876 | ND4 | | | | | | | | | | | | | | | | 7 | | | | 1 |
| 10907 | ND4 | | | | | | | 1 | | | | | | | | | 2:U4 | | | | |
| 10915 | ND4 | | | | | | | 1 | 3 | | | 2:L1a | | | | | | | | | 1 |
| 10927 | ND4 | | | | | | | | | | | | | | | | 2 | | | | 1 |
| 10978 | ND4 | | | | | | | | | | 7 | | | | | | | | | | |
| 11002 | ND4 | | | | | | | | | 2 | | | | 3 | | | | | | | 6, 10 |
| 11065 | ND4 | | | | | | | | 2 | | | | | | | | | | | | 1 |
| 11176 | ND4 | | | | | | | | | | | 2:L1a | | 1 | | | 1 | | | | |
| 11177 | ND4 | | | 14 | | | | | | | | | | | | | | | | | |
| 11197 | ND4 | | | | | | | | | | | | | | | | 2 | | | | 1 |
| 11242 | ND4 | | | | | | | | | | | | | | | 2 | | | | | |
| 11251 | ND4 | | | | | | | | | 33 | | | | | | 46 | | | | | 1 |
| 11299 | ND4 | | | | | | | | | | 47 | | | | | | | | | | 1 |
| 11314 | ND4 | | 2 | | | | | | | | | | | | | | | | | | |
| 11332 | ND4 | | | | | | | | | | | | | | | | 5:U4 | | | | 1, 4 |
| 11353 | ND4 | | | | | | | 2 | | | | | 1 | | | | | | | | |
| 11377 | ND4 | | | | | | | | | 3 | 1 | | | | | | | | | | 1 |
| 11467 | ND4 | | | | | | | | | | 46 | | | | | | 42 | | | | 1 |
| 11470 | ND4 | | | | | | | | | | 11 | | | | | | | | | | 1 |
| 11485 | ND4 | | | | | | | | | | 7 | | | | | | | | | | |
| 11560 | ND4 | | | | | | | 2 | | | | | | | | | | | | | |
| 11593 | ND4 | A > T | | | | 2 | | | | | | | | | | | | | | | |
| 11641 | ND4 | | | | | | | | | | | 2:L1a | | | | | | | 1 | | 3 |
| 11654 | ND4 | | | | | | | | | | | 2 | | | | | | | | | |
| 11674 | ND4 | | | | | | | | | | | | | | | | | | 8 | | 1 |
| ##### | ND4 | | 25 | 18 | 13 | 9 | 3 | 6 | 14 | 33 | 47 | 13 | 23 | 20 | 1 | 46 | 42 | | 8 | 11 | |
| 11732 | ND4 | | | | | | | | | | | | | | | | 2 | | | | 1 |
| 11800 | ND4 | | | | | | | 2 | | | | | | 3 | | | | | | | |
| 11812 | ND4 | | | | | | | | | | | 1:a | | | | 35 | | | | | 1 |
| 11840 | ND4 | | | | | | | | | | 7 | | | | | | | | | | |
| 11869 | ND4 | C > A | | | | | | | | | 5 | | | | | | | | | | |
| 11899 | ND4 | | | | | | | | | | | 2 | | | | | | | | | |
| 11914 | ND4 | | | | 12 | | | 3 | | | 13 | 3 | 18 | 1 | | 2 | | | | | |
| 11923 | ND4 | | | | | | | | | | 3 | | | | | | | | | | |
| 11944 | ND4 | | | | | | | | | | | | 22 | | | | | | | | |
| 11947 | ND4 | | | | | | | | | | | | | | | | | | 8 | | 1 |
| 12007 | ND4 | | 22 | | 1 | | | | | 2 | | 2:L1a | | | | 1 | | | | | |
| 12049 | ND4 | | | | | | | | | | | 2 | | | | | | | | | |
| 12127 | ND4 | | | | | | | | | 2 | | | | | | | | | | | |
| 12236 | tRNA-S | | | | | | | | | | | | 3 | | | | | | | | |
| 12308 | tRNA-L | | | | | | | | | | 47 | | | | | | 42 | | | | 1, |
| | | | | | | | | | | | | | | | | | | | | | 3-5, |
| | | | | | | | | | | | | | | | | | | | | | 9 |
| 12346 | ND5 | | | | | | 1 | 1 | | 1 | | | | | | | 2 | | | | |
| 12372 | ND5 | | | | 1 | | | | | | 47 | | | | | | 42 | | | | 1, 4 |
| 12414 | ND5 | | | | 1 | | | | | | | | | 1 | | | | | 8 | | |
| 12468 | ND5 | | 4 | | | | | | | | | | | | | | | | | | |
| 12501 | ND5 | | | | | | | | 14 | | | | | | | | | | | | 1 |
| 12519 | ND5 | | | | | | | | | | | 7 | | | | | | | | | |
| 12557 | ND5 | | | | | | | | | | | | | | | | 2 | | | | |
| 12570 | ND5 | | | | | | | | | 2 | | | | | | | | | | | |
| 12579 | ND5 | | | | | | | 2 | | | | | | | | | | | | | |
| 12612 | ND5 | | | | | | | | | 33 | | | | | | | | | | | 1, 4 |
| 12618 | ND5 | | | | | | | | | | | | | | | | 2 | | | | 1 |
| 12630 | ND5 | | | | | | | 2 | | | | | 3 | | | | | | | | |
| 12633 | ND5 | C > A | | | | | | | | | | | | | | 11 | | | | | 1 |
| 12642 | ND5 | | | | 2 | | | 2 | | | | | | | | | | | | | |
| 12693 | ND5 | | | | | | | | | | | | 18 | | | | | | | | |
| 12705 | ND5 | | 25 | | 13 | 9 | 3 | | 14 | | | 13 | 23 | 19 | 1 | | | | 8 | 11 | 1, 4 |
| 12720 | ND5 | | | | | | | | | | | 2:L1a | | | | | | | | | |
| 12810 | ND5 | | | | | | | | | | | 4 | | | | | | | | | 3, 8, |
| | | | | | | | | | | | | | | | | | | | | | 10 |
| 12811 | ND5 | | 2 | | | | | | | | 1 | | | | | | | | | | |
| 12948 | ND5 | | | | | | | | | | | | 3 | | | | | | | | |
| 12954 | ND5 | | | | | | | | | | 7 | | | | | | | | | | |
| 12978 | ND5 | | | | 3 | | | | | | | | | | | | | | | | |
| 13020 | ND5 | | | | | | | 2 | | | | | | | | | 7 | | | 1 | 1 |
| 13101 | ND5 | A > C | | | | | | | | | | | | 2 | | | | | | | |
| 13105 | ND5 | | | | | | | 2 | | | | 13 | | 10 | | | | 1 | | | |
| 13135 | ND5 | | | | | | | | | | 5 | | | | | | | | | | |
| 13149 | ND5 | | | | | | | | | | | 2 | | | | | | | | | |
| 13263 | ND5 | | | | 12 | | | | | | | | | | | | | | 4 | | 3, 4, |
| | | | | | | | | | | | | | | | | | | | | | 5, 11, |
| | | | | | | | | | | | | | | | | | | | | | 13-17 |
| 13276 | ND5 | | | | | | | | | | | 2:L1a | | | | | | | | | |
| 13281 | ND5 | | | | | | | | | 3 | | | | | | | | | | | |
| 13368 | ND5 | | | | | | | | | | | | | | | 46 | | | | | 1, |
| | | | | | | | | | | | | | | | | | | | | | 3-5, |
| | | | | | | | | | | | | | | | | | | | | | 9 |
| 13404 | ND5 | | | | | | | 4 | | | | | | | | | | | | | |
| 13485 | ND5 | | | | | | | | | | | 4 | | | | | | | | | |
| 13506 | ND5 | | | | | | | | | | | 13 | | | | | | | | | |
| 13590 | ND5 | | | 17 | | | | | | | | | 23 | | | | | | | | |
| 13617 | ND5 | | | | | | | | | | | | | | | | 23 | | | | 1 |
| 13637 | ND5 | | | | | | | | | | | | | | | | 4 | | | | 1 |
| 13650 | ND5 | | | | | | | | | | | 13 | 23 | | | | | | | | |
| 13680 | ND5 | | | | | | | 2 | | | | | | | | | | | | | |
| 13681 | ND5 | | | | | | | | | 2 | | | | | | | | | | | |
| 13708 | ND5 | | 3 | 1 | | | | 4 | | 33 | | | 3 | | | | 1 | | | 5 | 1, |
| | | | | | | | | | | | | | | | | | | | | | 3-5, |
| | | | | | | | | | | | | | | | | | | | | | 9, 12, |
| | | | | | | | | | | | | | | | | | | | | | 14 |
| 13734 | ND5 | | | | | | | | | | | | | | | 2 | 7 | | | | 1 |
| 13740 | ND5 | | | | | | | | | | 7 | | | | | | | | | | |
| 13758 | ND5 | C > A | | | | | | | | | | | | | | 2 | | | | | |
| 13759 | ND5 | | | | | | | 3 | | | | | | | | | | | | | |
| 13780 | ND5 | | | | | | | | 13 | | | | | | | | | | | | 1 |
| 13789 | ND5 | | | | | | | | | | | 11 | | | | | | | | | |
| 13803 | ND5 | | | | | | | | | | | | 18 | | | | | | | | 2, 10 |
| 13855 | ND5 | | 2 | | | | | | | | | | | | | | | | | | |
| 13879 | ND5 | | | | | | | | | 2 | | | | | | | | | | | 1 |
| 13880 | ND5 | C > A | | | | | | | | | | 7 | | | | | | | | | |
| 13886 | ND5 | | | | | | | | | | 1 | | | 3 | | | | | | | |
| 13914 | ND5 | C > A | | | | | | | | | | | | 6 | | | | | | | |
| 13933 | ND5 | | | | | | | | | 3 | | | | | | | | | | | |
| 13934 | ND5 | | | | | | | | | 5 | | | | | | | 3 | | | | |
| 13965 | ND5 | | | | | | | | | | | | | | | 8 | | | | | |
| 13966 | ND5 | | | | | | | | | | | | | | | 3 | | | | 11 | 1 |
| 13967 | ND5 | | | | | | | | | | 3 | | | | | | | | | | |
| 13980 | ND5 | | | | | | 1 | | | | | 2 | | | | | | | | | |
| 14000 | ND5 | T > A | | | | | | | | | | 4 | | | | | | | | | |
| 14002 | ND5 | | | | | | | | | | 2 | | | | | | | | | | |
| 14037 | ND5 | | | | | | | | | | 5 | | | | | | | | | | |
| 14040 | ND5 | | | | | | | | | | 2 | | | | | | | | | | |
| 14059 | ND5 | | | | | | | | | | | | 3 | | | | | | | | |
| 14125 | ND5 | | | | | | | 2 | | | | | | | | | | | | | |
| 14139 | ND5 | | | | | | | | | | | | | | | | 3 | | | | |
| 14148 | ND5 | | | | | | | | | | | 2 | | | | | | | | | |
| 14152 | ND6 | | | | | | | | | | | | | 3 | | | | | | | |
| 14167 | ND6 | | | | | | | | | | 47 | | | | | | | | | | 1 |
| 14178 | ND6 | | | | | | | | | | | 11 | | | | | | | | | |
| 14179 | ND6 | | | | | | | | | | | | | | | | 2 | | | | |
| 14182 | ND6 | | | | | | 1 | | 1 | | | | | | | | 8 | | | | 1 |
| 14203 | ND6 | | | | | | | | | | | 6 | | | | | | | | | |
| 14212 | ND6 | | | | | | | 2 | | | | | | 11 | | | | | | | |
| 14233 | ND6 | | | | | | | | 1 | | | | | | | 35 | | | | | 1 |
| 14284 | ND6 | | | | | | | | | | | | | 3 | | | | | | | |
| 14308 | ND6 | | | | | | 1 | | | | 1 | 2:L1a | | | | | | | | | |
| 14318 | ND6 | | | | 12 | | | | | | | | | | | | | | | | |
| 14350 | ND6 | | | | | | | 2 | | | | | | | | | | | | | |
| 14364 | ND6 | | 4 | | | | | 1 | | | | | | | | | 1 | | | | |
| 14365 | ND6 | | | | | | | 9 | | | | | | | | | | | | | |
| 14470 | ND6 | | 1 | | 1 | | | 1 | | | | | | | | | 1 | | | 11 | 1, 4 |
| 14470 | ND6 | T > A | | | | | | 3 | | | | | | | | | | | | | |
| 14544 | ND6 | | | | | | | | | | | | 2 | | | | | | | | 1 |
| 14560 | ND6 | | | | | | | | 1 | | | 11 | | | | | | | | | |
| 14566 | ND6 | | | | | | | | | | | | 17 | | | | | | | | |
| 14569 | ND6 | | | | | | | 3 | | 2 | 1 | | | | | | | | | | |
| 14582 | ND6 | | | | | | | 10 | | | | | | | | | | | | | |
| 14599 | ND6 | | | | | | | | | | | | 2 | | | | | | | | |
| 14620 | ND6 | | | | | | | | | | | | | | | | 5:U4 | | | | 1 |
| 14668 | ND6 | | | | | 9 | | | | | | | | | | | | | | | |
| 14687 | tRNA-E | | | | | | | | | | | | | | | 8 | | | | | |
| 14766 | CYT B | | 25 | 17 | 13 | 9 | 3 | 2 | 11 | 33 | 46 | 13 | 23 | 20 | 1 | 46 | 42 | | 8 | 11 | 3 |
| 14769 | CYT B | | | | 1 | | | | | | | 6 | | | | | 1 | | | | |
| 14783 | CYT B | | | | 13 | 9 | 3 | | | | | | | | 1 | | | | | | |
| 14793 | CYT B | | | | | | | | | | | | | | | | 16 | | | | 1 |
| 14798 | CYT B | | | | | | | | | 24 | 47 | | | | | | | | | | 1 |
| 14866 | CYT B | | | | | | | | | | | | | | | | 2:U4 | | | | |
| 14872 | CYT B | | | | | | | 2 | | | | | | | | | | | | | |
| 14905 | CYT B | | | | | | | | | | | | | 3 | | 46 | | | | | 1, 4 |
| 14911 | CYT B | | | | | | | | | | | 4 | | | | | | | | | |
| 15028 | CYT B | | | | | | | | | | | | | | | 5 | | | | | 1 |
| 15043 | CYT B | | | | 13 | 9 | 3 | | 13 | | | | | | 1 | 5 | | | | | 1, 4 |
| 15099 | CYT B | | | | | | | | | | | 1 | | 2 | | | | | | | |
| 15110 | CYT B | | | | | | | 1 | | | | | 3 | 1 | | | | | | | |
| 15115 | CYT B | | | | | | | 1 | | | | 7 | | | | | | | | | |
| 15136 | CYT B | | | | | | | | | | | 2:L1a | | | | | | | | | |
| 15191 | CYT B | | | | | | | | | | | | | | | | 2 | | | | |
| 15217 | CYT B | | | | | | | | | | | | 3 | | | | | | | | |
| 15218 | CYT B | | | | | | | 2 | | | | | | | | | 13 | | | | 1 |
| 15229 | CYT B | | | | | | | | | | | | 2 | | | | | | | | |
| 15236 | CYT B | | | | | | | | | | | | 2 | | 1 | | | | | | |
| 15244 | CYT B | | | 1 | | | | | | | | | 7 | | | | | | | | |
| 15257 | CYT B | | | | | | | | | 6 | 3 | | | | | | | | | | 1, 14 |
| 15274 | CYT B | | | | | | | | | | | | | | | 2 | | | | | |
| 15301 | CYT B | | | 1 | 13 | 9 | 3 | | | | 1 | 1 | 23 | 20 | 1 | | 1 | | | | |
| 15311 | CYT B | | | | | | | | | | | | | 6 | | | | | | | |
| 15326 | CYT B | | 25 | 18 | 13 | 9 | 3 | 220 | 14 | 33 | 47 | 13 | 21 | 20 | 1 | 46 | 42 | 8 | 8 | 11 | |
| 15391 | CYT B | | | | | | | | | | | | 2 | | | | | | | | |
| 15452 | CYT B | C > A | | | | | | | | 33 | | | | | | 46 | | | | | 1 |
| 15454 | CYT B | | | | | | | | | | | | | | | | 3 | | | | |
| 15466 | CYT B | | | | | | | 2 | | | | | | | | | | | | | |
| 15487 | CYT B | A > T | | | 12 | | | | | | | | | | | | | | | | |
| 15535 | CYT B | | | 17 | | | | | | | | | | | | | | | | | |
| 15607 | CYT B | | | | | | | | | | | | | | | 46 | | | | | 1, 4, |
| | | | | | | | | | | | | | | | | | | | | | 5, 9 |
| 15629 | CYT B | | | | | | | | | | | | 7 | | | | | | | | |
| 15670 | CYT B | | 1 | | 2 | | | | | | | | | 5 | | | | | | | |
| 15679 | CYT B | | | | | | | | | 2 | | | | | | | | | | | |
| 15693 | CYT B | | | | | | | | | | | | | | | | 5:U4 | | | | 1 |
| 15758 | CYT B | | | | | | | | 6 | | | 1 | | | | 1 | | | | | 1 |
| 15775 | CYT B | | | | | | | | | | | | | | | | | 1 | 2 | | |
| 15784 | CYT B | | | 1 | | | | 1 | | | 1 | 1 | 17 | | | | 3 | | 4 | | |
| 15789 | CYT B | | | | | | | 2 | | | | | | | | | | | | | |
| 15808 | CYT B | | | | | | | 2 | | | | | | | | | | | | | |
| 15812 | CYT B | | | | | | | | | 3 | | | | 1 | | | | | | | 1 |
| 15824 | CYT B | | | | | | | | | | | | | 6 | | | | | | | |
| 15833 | CYT B | | | | | | | 9 | | | | | | | | | | | | | 1 |
| 15884 | CYT B | | | 1 | | | | 3 | | | 4 | | | | | | | | | | |
| 15884 | CYT B | G > C | | | | | | | | | | | | | | | | | 8 | | 1 |
| 15904 | tRNA-T | | | | | | | | | | | | | | | | | 8 | | | 1, 4 |
| 15907 | tRNA-T | | | | | | | | | | | | | | | | 7 | | | | 1, 4 |
| 15924 | tRNA-T | | | | | | | 3 | 11 | 1 | 12 | | | | | | 2 | | 1 | | 1, 4 |
| 15927 | tRNA-T | | | | | | | | | | | | | | | | | | | 5 | 1 |
| 15928 | tRNA-T | | | | | | | | | | | | | | | 46 | | | | | 1, 4, |
| | | | | | | | | | | | | | | | | | | | | | 9 |
| 15930 | tRNA-T | | | | 6 | | | 2 | | | | | | | | | 1 | | | | |
| 15942 | tRNA-T | | | | | | | | | | | | | 4 | | | | | | | |
| 15944 | tRNA-T | DEL | | | | | | | | | | | | 6 | | | | | | | |
| 15945 | tRNA-T | T-INS | | | | | | | | | | | 2 | | | | | | | | |
| 15946 | tRNA-T | | | | | | | | | | 2 | | | | | | | | | | |
| 16051 | D-LOOP | | | | 1 | | | 5 | | | | | | 1 | | 1 | 6 | | | | |
| 16069 | D-LOOP | | | | | | | | | 23 | | | | | | | | | | | |
| 16092 | D-LOOP | | 3 | 2 | | | | | | 2 | 1 | | | | | | | | | | |
| 16111 | D-LOOP | | 23 | 3 | | | | | | | | | 1 | | | | | | | | |
| 16114 | D-LOOP | C > A | | | | | | | | | | | 3 | | | | 1 | | | | |
| 16124 | D-LOOP | | | | | | | | | | | | | 7 | | | | | | | |
| 16126 | D-LOOP | | | | | | | 2 | | 23 | | 6 | | | | 38 | | | | | |
| 16148 | D-LOOP | | | | | | | | | | | 2:L1a | | | | | | | | | |
| 16162 | D-LOOP | | | | | | | 14 | | | | | | | | 1 | | | | | |
| 16163 | D-LOOP | | | | | | | | | | | | | | | 7 | | | | | |
| 16172 | D-LOOP | | | | | | | 2 | 2 | 1 | | 3 | | 2 | | | 2 | | | | |
| 16182 | D-LOOP | A > C | | 8 | | | | 1 | | | | | | 1 | | 5 | | | | | |
| 16186 | D-LOOP | | | | | | | | | | | | | | | 8 | | | | | |
| 16187 | D-LOOP | | | | | | | | | | | 11 | | | | | | | | | |
| 16188 | D-LOOP | C > G | | | | | | 1 | | | | 2:L1a | | | | | | | | | |
| 16213 | D-LOOP | | | | | | | | | | | | 3 | | | | | | | | |
| 16217 | D-LOOP | | | 14 | | | | | | | | | | | | | | | | | |
| 16224 | D-LOOP | | | | | 1 | | | | | 41 | | | | | | | | | | |
| 16230 | D-LOOP | | | | | | | | | | | 2:L1a | | | | | | | | | |
| 16234 | D-LOOP | | 1 | | 2 | | 1 | 2 | | 1 | 6 | | | 2 | | | | | 1 | | |
| 16256 | D-LOOP | | | | | | | | | | | | | | | | 17 | 1 | | | |
| 16261 | D-LOOP | | | 1 | | | 1 | 2 | | 4 | | | | | | | | | | | |
| 16264 | D-LOOP | | | | | | | 2 | | | | 6 | | 1 | | | | | | | |
| 16265 | D-LOOP | | | | | | | | | | | 2 | | | | | | | | | |
| 16270 | D-LOOP | | 1 | | | | | | | | 2 | 4 | | | | | 18 | | | | |
| 16278 | D-LOOP | | 1 | 1 | | | | 4 | | 1 | | 11 | 23 | 6 | | | 2 | | | 10 | |
| 16290 | D-LOOP | | 23 | | | | | | | | 1 | | | | | | | | | | |
| 16291 | D-LOOP | | 1 | | | | | 8 | | | | 1 | | 1 | | | 4 | | | | |
| 16292 | D-LOOP | | | | | | | | | | | 1 | 1 | | | 1 | | | 7 | | |
| 16293 | D-LOOP | | | | | | | 8 | | | | 5 | | | | | | | | | |
| 16294 | D-LOOP | | | | | | | 2 | | | | 4 | 17 | 1 | | 38 | 2 | | | | |
| 16296 | D-LOOP | | | | | | | 1 | | | | | | | | 27 | | | | | |
| 16298 | D-LOOP | | | 1 | 12 | | | 3 | | | | | | | | 5 | | 3 | | | |
| 16304 | D-LOOP | | | | | | | 16 | 1 | | 1 | | | | | 9 | | | | | |
| 16309 | D-LOOP | | | | | | | 1 | | | 1 | | 13 | | | | | | | | |
| 16311 | D-LOOP | | | | 1 | | 1 | 14 | 2 | 1 | 43 | 12 | 1 | 7 | | 1 | 3 | 1 | 1 | | |
| 16319 | D-LOOP | | 25 | 1 | 1 | | | | | 2 | 3 | | | | | | | | | | |
| 16320 | D-LOOP | | | | | | | 2 | 1 | | 2 | 2:L1a | | 3 | | | | | | | |
| 16324 | D-LOOP | | | | | | | 1 | | | | | | | | 5 | | | | | |
| 16325 | D-LOOP | | 1 | 1 | 10 | 9 | | 1 | | | | | | | | | | | | | |
| 16327 | D-LOOP | | | | 12 | | | | | | | | | 5 | | | | | | | |
| 16356 | D-LOOP | | | | | | | 9 | | | | | | | | | 4 | | | | |
| 16360 | D-LOOP | | | | | | | | | | | 4 | | | | | | | | | |
| 16362 | D-LOOP | | 24 | | 2 | 9 | 2 | 14 | | 1 | 1 | | 3 | 6 | 1 | | 3 | | 1 | | |
| 16390 | D-LOOP | | 1 | | 1 | 1 | 1 | | 2 | 1 | | | 22 | | | | 3 | 1 | | | |
| 16391 | D-LOOP | | 1 | | | | | | 10 | | | | | | | | | | | | |
| 16399 | D-LOOP | | | | | | | | | | | 1 | 2 | 1 | | 2 | 10 | | | | |
| 16463 | D-LOOP | | | | | | | | | | 2 | | | | | | | | | | |
| 16482 | D-LOOP | | | | | | | 9 | | | | | | | | | | | | | |
| 16527 | D-LOOP | | | | 1 | | | | | | | 2 | | | | 1 | | | | | |

TV/DEL/INS column: all nucleotide substitutions are transitions unless indicated otherwise
NP: nucleotide position;
TV: transversion;
DEL: deletion;
INS: insertion
References
1: Finnilä et al. 2001 Am J Hum Genet 68:1475-1474
2: Chen et al. 2000 Am. J. Hum. Genet. 66:1362-1383
3: Alves-Silva et al. 2000 Am. J. Hum. Genet. 67:444-461
4: Macaulay et al. 1999 Am J Hum Genet 64:232-249
5: Wallace et al. 1999 Gene 238:211-230
6: Quintana-Murci et al. 1999Nat Genet 23:437-41
7: Lehtonen et al. 1999s in mtDNA
8: Rando et al. 1998Am J Hum Genet 62:531-550
9: Torroni et al. 1996. Genetics 144:1835-1850
10: Chen et al. 1995 Am J Hum Genet 57:133-149
11: Torroni et al. 1994 Am J Phys Anthropol 93:189-99
12: Torroni et al. 1994 Am J Hum Genet 55:760-776
13: Torroni et al. 1994 Am. J. Phys. Anthr. 93:189-199
14: Torroni et al. 1994 J Bioener Biomembr 26:261-71
15: Torroni et al. 1993 Am J Hum Genet 53:563-590
16: Torroni et al. 1992 Genetics 130:153-162
17: Wallace et al. 1992 Hum Biol 64:403-416

A mitochondrial single nucleotide polymorphism or homoplasmic mtDNA point mutation, which includes a deviation in the identity of the nucleotide base situated at a specific position in a mtDNA sequence relative to the “wildtype” human mtDNA sequence (CRS) disclosed by Anderson et al. (1981), may fall into at least one of the following categories: An “error” refers to sequencing mistakes in the human mtDNA sequence reported by Anderson et al. (1981), as corrected by Andrews et al. (1999 Nature Genetics 23:147). A “polymorphism” refers to a known polymorphism in a human mtDNA sequence that is not associated with a particular human disease, but that has been detected and described as a result of naturally occurring variability in the identity of the nucleotide base situated at a given position in a human mtDNA sequence (see, e.g., “Mitomap”, Emory University School of Medicine, available at http://www.gen.emory. edu/mitomap.html). A “rare polymorphism” refers to a mtDNA nucleotide that differs from the base situated at the corresponding position in the Cambridge Reference Sequence (CRS) of Anderson et al. (1981) but which, upon subsequent accumulation of human mtDNA sequence data from a plurality of subjects (and in contrast to the reliance of Anderson et al. upon the mtDNA sequence of a single donor to generate the CRS), suggests the presence of a low frequency allele in the CRS donor, relative to the larger sample population (see Andrews et al., 1999 Nature Genetics 23:147 and references cited therein). Particularly useful mutations that segregate with AD according to the present invention include homoplasmic mtDNA point mutations (e.g., single nucleotide polymorphisms) that are not errors, polymorphisms or rare polymorphisms as just described, and additionally, the homoplasmic mtDNA point mutations (e.g., single nucleotide polymorphisms). A number of polymorphisms are identified in Tables 3 and 4, and in FIGS. 1-6; full length mtDNA sequences of 560 unrelated human subjects are set forth at SEQ ID NOS:2-561 in the Sequence Listing.

In certain embodiments of the invention, the presence or absence of a specific genetic mutation or variation, such as, for example, a single nucleotide polymorphism or a deletion, that correlates with a specific haplogroup, disease or individual may be sufficient to determine the haplogroup, presence or risk of disease or identity of the individual from whom the biological sample being tested was obtained. In these situations, the association or correlation of a particular genetic mutation or variation with a haplogroup, disease or individual may be determined by means generally acceptable to those with skill in the relevant or a related art. For example, an association or correlation may be established by the presence of a statistically significant increase or decrease in the presence or absence of a single nucleotide polymorphism or other genetic alteration or marker in samples from subjects with a disease or haplogroup. An association or correlation with a specific indivudal may also be determined by a statistically significant presence or absence of a specific genetic mutation or alteration within mtDNA derived from the individual compared to the general population or a sample of other individuals.

In other embodiments of the invention, the determination of the presence or risk of a disease, the haplogroup or identity of an individual, or the genetic relationship between individuals may be determined by analyzing one or more genetic markers, including one or more mtDNA single nucleotide polymorphisms or deletions. In these situations, the correlation or association of one specific mtDNA mutation or alteration with a certain phenotype or individual need not be statistically significant in isolation. Rather, the overall analysis of multiple markers may be used to establish the association or correlation. The presence or absence of one or more markers together may be statistically associated or correlated with a disease, haplogroup or individual.

As presented below, the accompanying examples reveal that among individuals of the general population in the United States there are large differences in the frequencies of single nucleotide polymophisms (typically nucleotide substitutions) that are carried in the mtDNA genome (see also Table 3). While distinct sets of SNPs are associated with particular mtDNA haplogroups, individuals in the examples described below were found to carry up to 8 additional non-synonymous and 22 additional synonymous nucleotide substitutions in protein-coding genes, up to 8 nucleotide changes each in the ribosomal RNA and tRNA genes, and up to 13 changes in the D-loop region. Most of these nucleotide substitutions are likely to be of systemic nature as suggested by their detection following analyses of paired blood and skeletal muscle samples. Additionally, while the samples collected in the attached examples were non-neoplastic tissues, they exhibited some of the same nucleotide changes that were reported as acquired mutations in cancer tissue (Fliss, M. S. et al. (1999) Science 287:2017-2019). Thus, as also noted above and as shown in Table 3, the present invention provides non-haplogroup associated mitochondrial single nucleotide polymorphisms that may be used to determine the unique identity of an individual and/or to determine the presence of or risk for having a disease,(e.g., Alzheimer's disease, diabetes), in addition to providing improved profiles of mitochondrial single nucleotide polymorphisms for identifying a mitochondrial haplogroup and/or a mitochondrial haplogroup subgroup.

The present invention also contemplates compositions and methods for the detection of potentially pathogenic mtDNA mutations involved in human disease, either in the background of, or independent of, polymorphisms associated with mtDNA haplogroups. As noted above, the invention also provides materials and methods for haplogroup identification and genealogical and forensic analyses.

The following examples are offered by way of illustration, and not by way of limitation.

### EXAMPLES

### Example 1

### DNA Isolation From Blood, Muscle and Brain Samples

Blood samples, muscle biopsies, and frozen brain samples were collected from 560 maternally unrelated individuals (as determined from family-history information) of European, African and Asian descent after institutional review board (IRB) approval and informed consent. The sampled population consisted of 38% females and 62% males ranging in age from 33-93 years and 24-103 years with mean ages of 72 and 61, respectively. Total cellular DNA was prepared from white blood cells and frozen brain tissue by homogenization and cell lysis in TE buffer (10 mM Tris-HCl, pH 7.5, 1 mM EDTA) containing proteinase K (400μ/ml) and 1% SDS at 37° C. for 12 hr, followed by phenol:chlorofom:isoamyl alcohol (50:48:2) and chloroform:isoamyl alcohol (24:1) extractions. Alternatively, mitochondria were isolated from frozen brain tissue and mtDNA was extracted. DNA was precipitated with ethanol and resuspended in TE buffer. DNA concentrations were determined by UV absorption at 260 nm.

### Example 2

PCR Amplification, Sequencing and Sequence Analysis

MtDNA was amplified in 68 fragments of approximately 500 bp each in length with 50% overlap between neighboring fragments. PCR primers were 16-26 nucleotides in length and designed to be complementary to the mitochondrial light and heavy strands.

PCR amplification was performed as described below using the oligonucleotide primer set presented in Table 5 was used to generate 68 PCR product fragments spanning the complete mtDNA molecule, each fragment having approximately 50% sequence overlap with each neighboring product fragment. This strategy permitted direct mtDNA sequencing in both forward and reverse directions with four-fold redundancy in the identification of each nucleotide base, resulting in error-free sequencing. Thus, for each patient sample approximately 68,000 nucleotides were sequenced and analysis of homoplasmic mutations was verified.

TABLE 5

OLIGONUCLEOTIDE PRIMERS SPECIFIC FOR INDICATED REGIONS OF MITOCHONDRIAL GENOME

| | | | | | | | |
|---|---|---|---|---|---|---|---|
| | | | | PRIM. | | SEQ | FRAG. |
| FRAGMENT | PRIMER | GENE | NUCLEOTIDE | LENGTH | PRIMER SEQUENCE 5′ −> 3′ | ID NO. | LENGTH |

| | | | | | | | |
|---|---|---|---|---|---|---|---|
| 201 | 201F | D-Loop | 16225L | 24 | CAACTATCACACATCAACTGCA | | 512 |
| | | | | | AC | | |
| | 201R | | 168H | 21 | TCGCCTGTAATATTGAACGTA | | |
| 202 | 202F | D-Loop | 16477L | 23 | GCTAAAGTGAACTGTATCCGA | | 379 |
| | | | | | CA | | |
| | 202R | | 287H | 21 | GACTGTTAAAAGTGCATACCG | | |
| 203 | 203F | D-Loop | 155L | 21 | TATTTATCGCACCTACGTTCA | | 407 |
| | 203R | | 562H | 18 | AACTGTGGGGGGTGTCTT | | |
| 204 | 204F | D-Loop/tRNA | 275L | 23 | GACATGATAACAAAAAATTTC | | 510 |
| | | Phe/12S | | | CA | | |
| | 204R | | 785H | 18 | GTGTGGCTAGGCTAAGCG | | |
| 205 | 205F | D-Loop/tRNA | 498L | 16 | CGCCCATCCTACCCAG | | 541 |
| | | Phe/12S | | | | | |
| | 205R | | 1039H | 25 | TCTTAGCTATTGTGTGTTCAGA | | |
| | | | | | TAT | | |
| 206 | 206F | 12S | 773L | 19 | TGCAGCTCAAAACGCTTAG | | 525 |
| | 206R | | 1298H | 18 | CGTGGGTACTTGCGCTTA | | |
| 207 | 207F | 12S | 1031L | 24 | GCTTTAACATATCTGAACACAC | | 505 |
| | | | | | AA | | |
| | 207R | | 1536H | 24 | CTTGTCTCCTCTATATAAATGC | | |
| | | | | | GT | | |
| 208 | 208F | 12S/tRNA | 1285L | 20 | GAAGGCTACAAAGTAAGCGC | | 500 |
| | | Val/16S | | | | | |
| | 208R | | 1785H | 17 | TCATCTTTCCCTTGCGG | | |
| 209 | 209F | 12S/tRNA | 1535L | 24 | TACGCATTTATATAGAGGAGA | | 461 |
| | | Val/16S | | | CAA | | |
| | 209R | | 1996H | 18 | ATCACCAGGCTCGGTAGG | | |
| 210 | 210F | 16S | 1780L | 19 | TAGTACCGCAAGGGAAAGA | | 417 |
| | 210R | | 2197H | 19 | GTTGAGCTTGAACGCTTTC | | |
| 211 | 211F | 16S | 1986L | 17 | AGGCGACAAACCTACCG | | 411 |
| | 211R | | 2397H | 23 | GTGAGGGTAATAATGACTTGTT | | |
| | | | | | G | | |
| 212 | 212F | 16S | 2165L | 21 | CCATAGTAGGCCTAAAAGCAG | | 420 |
| | 212R | | 2585H | 22 | AGTGATTATGCTACCTTTGCAC | | |
| 213 | 213F | 16S | 2380L | 24 | CAATATCTACAARCAACCAAC | | 413 |
| | | | | | AAG | | |
| | 213R | | 2793H | 20 | ACCGAAATTTTTAATGCAGG | | |
| 214 | 214F | 16S | 2580L | 19 | TAACCGTGCAAAGGTAGCA | | 405 |
| | 214R | | 2985H | 18 | CCTGATCCAACATCGAGG | | |
| 215 | 215F | 16S | 2779L | 21 | CCTAAACTACCAAACCTGCAT | | 442 |
| | 215R | | 3221H | 20 | GCCATCTTAACAAACCTGT | | |
| 216 | 216F | 165/tRNA | 2974L | 19 | AGGGTTTACGACCTCGATG | | 517 |
| | | Leu/ND1 | | | | | |
| | 216R | | 3491H | 17 | GGTAGATGTGGCGGGTT | | |
| 217 | 217F | 16S/tRNA | 3228L | 19 | TTGTTAAGATGGCAGAGCC | | 506 |
| | | Leu/ND1 | | | | | |
| | 217R | | 3734H | 20 | AATGATGGCTAGGGTGACTT | | |
| 218 | 218F | ND1 | 3482L | 16 | AGCCCCTAAAACCCGC | | 498 |
| | 218R | | 3980H | 22 | ATAATGTTTGTGTATTCGGCTA | | |
| 219 | 219F | ND1 | 3718L | 23 | CAAACAATCTCATATGAAGTC | | 520 |
| | | | | | AC | | |
| | 219R | | 4238H | 17 | AGGGGGAATGCTGGAGA | | |
| 220 | 220F | ND1/tRNAs | 3967L | 19 | GCCCTATTCTTCATAGCCG | | 514 |
| | | Ile/Gln/Met/N | | | | | |
| | | D2 | | | | | |
| | 220R | | 4481H | 15 | ATGACGGGTTGGGCC | | |
| 221 | 221F | ND1/tRNAs | 4224L | 21 | CATACCCATTACAATCTCCAG | | 511 |
| | | Ile/Gln/Met/N | | | | | |
| | | D2 | | | | | |
| | 221R | | 4735H | 26 | TATTAATGATGAGTATTGATTG | | |
| | | | | | GTAG | | |
| 222 | 222F | ND2 | 4481L | 15 | GGCCCAACCCGTCAT | | 508 |
| | 222R | | 4989H | 20 | GCTAAGATTTTGCGTAGCTG | | |
| 223 | 223F | ND2 | 4691L | 23 | CCTCTTCAACAATATACTCTCC | | 548 |
| | | | | | G | | |
| | 223R | | 5239H | 16 | GGCAAAAAGCCGGTTA | | |
| 224 | 224F | ND2 | 4979L | 18 | AAACCAGACCCAGCTACG | | 506 |
| | 224R | | 5485H | 25 | AAGATTATTAGTATAAAAGGG | | |
| | | | | | GAGA | | |
| 225 | 225F | ND2/tRNAs | 5234L | 15 | CCCGCTAACCGGCTT | | 480 |
| | | Trp/Ala/Asn | | | | | |
| | 225R | | 5714H | 22 | GAGAAGTAGATTGAAGCCAGT | | |
| | | | | | T | | |
| 226 | 226F | ND2/tRNAs | 5455L | 17 | TCATCGCCCTTACCACG | | 537 |
| | | Trp/Ala/Asn/Cys/Tyr+ | | | | | |
| | | O-L | | | | | |
| | 226R | | 5992H | 19 | AGGAGGCTTAGAGCTGTGC | | |
| 227 | 227F | tRNAs | 5700L | 20 | TAAGCACCCTAATCAACTGG | | 542 |
| | | Ala/Asn/Cys/Tyr+ | | | | | |
| | | O-L/CO1 | | | | | |
| | 227R | | 6242H | 20 | CCTCCACTATAGCAGATGCG | | |
| 228 | 228F | COI | 5995L | 21 | CAGCTCTAAGCCTCCTTATTC | | 498 |
| | 228R | | 6493H | 21 | CAGCTAGGACTGGGAGAGATA | | |
| 229 | 229F | COI | 6230L | 19 | CCTACTCCTGCTCGCATCT | | 527 |
| | 229R | | 6757H | 21 | TATGGTGTGCTCACACGATAA | | |
| 230 | 230F | COI | 6476L | 24 | AGCAGTCCTACTTCTCCTATCT | | 510 |
| | | | | | CT | | |
| | 230R | | 6986H | 25 | GTCGTGTAGTACGATGTCTAGT | | |
| | | | | | GAT | | |
| 231 | 231F | COI | 6713L | 23 | CATAGGTATGGTCTGAGCTATG | | 517 |
| | | | | | A | | |
| | 231R | | 7230H | 18 | GGTGTATGCATCGGGGTA | | |
| 232 | 232F | CO1/tRNA | 6979L | 24 | CAAACTCATCACTAGACATCGT | | 501 |
| | | Ser | | | AC | | |
| | 232R | | 7480H | 17 | ATGGGGTTGGCTTGAAA | | |
| 233 | 233F | CO1/tRNAs | 7225L | 16 | CGGACTACCCCGATGC | | 519 |
| | | Ser/Asp/COII | | | | | |
| | 233R | | 7744H | 20 | CCTGAGCGTCTGAGATGTTA | | |
| 234 | 234F | tRNAs | 7469L | 18 | CCCAAAGCTGGTTTCAAG | | 510 |
| | | Ser/Asp/COII | | | | | |
| | 234R | | 7979H | 18 | GTCAAGGAGTCGCAGGTC | | |
| 235 | 235F | COII | 7690L | 20 | CTTCCTAGTCCTGTATGCCC | | 557 |
| | 235R | | 8247H | 18 | GGGTAAATACGGGCCCTA | | |
| 236 | 236F | COII/tRNA | 7975L | 16 | AGGCGACCTGCGACTC | | 504 |
| | | Lys/ATPase 8 | | | | | |
| | 236R | | 8479H | 21 | TTTATTTTTATGGGCTTTGGT | | |
| 237 | 237F | COII/tRNA | 8225L | 22 | ATTCCCCTAAAAATCTTTGAAA | | 516 |
| | | Lys/ATPase | | | | | |
| | | 8/ATPase 6 | | | | | |
| | 237R | 8741H | | | 25 | GCAATAAAAATGATTAAGGAT | |
| | | | | | ACTA | | |
| 238 | 238F | ATPase | 8499L | 24 | AAAATTATAACAAACCCTGAG | | 497 |
| | | 8/ATPase 6 | | | AAC | | |
| | 238R | | 8996H | 16 | GCGGTTAGGCGTACGG | | |
| 239 | 239F | ATPase | 8722L | 26 | CGAACCTGATCTCTTATACTAG | | 515 |
| | | 8/ATPase | | | TATC | | |
| | | 6/COIII | | | | | |
| | 239R | | 9237H | 18 | TCATGGGCTGGGTTTTAC | | |
| 240 | 240F | ATPase | 8978L | 18 | TTCAACCAATAGCCCTGG | | 506 |
| | | 6/COIII | | | | | |
| | 240R | | 9484H | 19 | CAGAAAAATCCTGCGAAGA | | |
| 241 | 241F | COIII | 9229L | 22 | ATCATATAGTAAAACCCAGCC | | 505 |
| | | | | | C | | |
| | 241R | | 9734H | 21 | TCGAAGTACTCTGAGGCTTGT | | |
| 242 | 242F | COIII/tRNA | 9470L | 21 | CTCAGAAGTTTTTTTCTTCGC | | 537 |
| | | Glu | | | | | |
| | 242R | | 10007H | 25 | AGTTAATTGGAAGTTAACGGT | | |
| | | | | | ACTA | | |
| 243 | 243F | COIII/tRNA | 9732L | 22 | CTACAAGCCTCAGAGTACTTCG | | 520 |
| | | Glu/ND3 | | | | | |
| | 243R | | 10252H | 21 | GGAGGGCAATTTCTAGATCAA | | |
| 244 | 244F | COIII/tRNA | 9976L | 24 | ATTGATGAGGGTCTTACTCTTT | | 523 |
| | | Glu/ND3/tRN | | | TA | | |
| | | A Arg | | | | | |
| | 244R | | 10499H | 23 | CTAGAAGTGAGATGGTAAATG | | |
| | | | | | CT | | |
| 245 | 245F | ND3/tRNA | 10209L | 26 | TTCTCCATAAAATTCTTCTTAG | | 522 |
| | | Arg/ND4L | | | TAGC | | |
| | 245R | | 10731H | 26 | AGTAGGTTTAGGTTATGTACGT | | |
| | | | | | AGTC | | |
| 246 | 246F | tRNA | 10450L | 24 | ATGATAATCATATTTACCAAAT | | 536 |
| | | Arg/ND4L/ND | | | GC | | |
| | | 4 | | | | | |
| | 246R | | 10986H | 18 | GTTGGCTTGCCATGATTG | | |
| 247 | 247F | ND4L/ND4 | 10719L | 23 | ACATATGGCCTAGACTACGTAC | | 511 |
| | | | | | A | | |
| | 247R | | 11230H | 18 | GCGATGAGTAGGGGAAGG | | |
| 248 | 248F | ND4 | 10979L | 17 | CCCCTCACAATCATGGC | | 502 |
| | 248R | | 11481H | 22 | AGTGTGAGGCGTATTATACCAT | | |
| 249 | 249F | ND4 | 11213L | 20 | TACACCCTAGTAGGCTCCCT | | 524 |
| | 249R | | 11737H | 21 | TTTGAGTTTGCTAGGCAGAAT | | |
| 250 | 250F | ND4 | 11474L | 20 | GGCGGCTATGGTATAATACG | | 510 |
| | 250R | | 11984H | 24 | CCATTGTGTTGTGGTAAATATG | | |
| | | | | | TA | | |
| 251 | 251F | ND4/tRNAs | 11721L | 24 | TTACATCCTCATTACTATTCTG | | 511 |
| | | His/Ser | | | CC | | |
| | 251R | | 12232H | 18 | TTAGACATGGGGGCATGA | | |
| 252 | 252F | ND4/tRNAs | 11968L | 23 | AGCCCTATACTCCCTCTACATA | | 538 |
| | | His/Ser/Leu/ND5 | | | T | | |
| | 252R | | 12506H | 25 | TTCGAGATAATAACTTCTTGGT | | |
| | | | | | CTA | | |
| 253 | 253F | tRNAs | 12213L | 22 | GCTCACAAGAACTGCTAACTC | | 516 |
| | | Ser/Leu/ND5 | | | A | | |
| | 253R | | 12729H | 22 | GAATAGGTTGTTAGCGGTAACT | | |
| 254 | 254F | ND5 | 12458L | 24 | CATCCACCTTTATTATCAGTCT | | 532 |
| | | | | | CT | | |
| | 254R | | 12990H | 17 | ATTTGCCTGCTGCTGCT | | |
| 255 | 255F | ND5 | 12714L | 25 | TACCATACTAATCTTAGTTACC | | 520 |
| | | | | | GCT | | |
| | 255R | | 13234H | 21 | AGTGGAGAAGGCTACGATTTT | | |
| 256 | 256F | ND5 | 12979L | 17 | GGCCTCCTCCTAGCAGC | | 501 |
| | 256R | | 13480H | 21 | ACCTGTGAGGAAAGGTATTCC | | |
| 257 | 257F | ND5 | 13225L | 21 | GACATCAAAAAAATCGTAGCC | | 504 |
| | 257R | | 13729H | 25 | AAATGTTGTTAGTAATGAGAA | | |
| | | | | | ATCC | | |
| 258 | 258F | ND5 | 13472L | 21 | CATTAGCAGGAATACCTTTCC | | 506 |
| | 258R | | 13978H | 19 | CTAGGAGGAGTAGGGGCAG | | |
| 259 | 259F | ND5/ND6 | 13720L | 21 | CTATTCGCAGGATTTCTCATT | | 517 |
| | 259R | | 14237H | 16 | GTGCGGGGGCTTTGTA | | |
| 260 | 260F | ND5/ND6 | 13939L | 19 | CGCACAATCCCCTATCTAG | | 545 |
| | 260R | | 14484H | 22 | TTAATTTATTTAGGGGGAATGA | | |
| 261 | 261F | ND6/tRNA | 14209L | 22 | AACTACTACTAATCAACGCCCA | | 522 |
| | | Glu | | | | | |
| | 261R | | 14731H | 22 | GGTCATTGGTGTTCTTGTAGTT | | |
| 262 | 262F | ND6/tRNA | 14471L | 20 | CCAAAGACAACCATCATTCC | | 508 |
| | | Glu/Cyt b | | | | | |
| | 262R | | 14979H | 18 | CGTGAAGGTAGCGGATGA | | |
| 263 | 263F | tRNA Glu/Cyt | 14715L | 23 | ACCATCGTTGTATTTCAACTAC | | 514 |
| | | b | | | A | | |
| | 263R | | 15229H | 21 | TAGCCTCCTCAGATTCATTGA | | |
| 264 | 264F | Cyt b | 14961L | 22 | ACGTAAATTATGGCTGAATCAT | | 518 |
| | 264R | | 15479H | 20 | CCTAGGAGGTCTGGTGAGAA | | |
| 265 | 265F | Cyt b | 15223L | 22 | CCTAGTTCAATGAATCTGAGGA | | 509 |
| | 265R | | 15732H | 17 | AATGAGGAGGTCTGCGG | | |
| 266 | 266F | Cyt b/tRNA | 15449L | 24 | TTCCTTCTCTCCTTAATGACATT | | 530 |
| | | Thr/Phe | | | A | | |
| | 266R | | 15979H | 20 | TAGCTTTGGGTGCTAATGGT | | |
| 267 | 267F | Cyt b/tRNAs | 15723L | 18 | GACTCCTAGCCGCAGACC | | 509 |
| | | Thr/Phe/D-Loop | | | | | |
| | 267R | | 16232H | 20 | GGAGTTGCAGTTGATGTGTG | | |
| 268 | 268F | tRNA Phe/D-Loop | 15968L | 22 | TCTTTAACTCCACCATTAGCAC | | 517 |
| | 268R | | 16485H | 24 | GGAACCAGATGTCGGATACAG | | |
| | | | | | TTC | | |

PCR amplifications were performed in triplicate, each containing 5-50 ng total cellular DNA or 1 ng mtDNA, 100 ng of each forward and reverse primers, and 12.5 μl of Taq PCR Master Mix (Qiagen) in a reaction volume of 25 μl. After denaturation at 95° C. for 2 min, amplification was carried out for 30 cycles at 95° C. for 10 sec, 60° C. for 10 sec, and 72° C. for 1 min, followed by 72° C. for 4 min and cooling to 4° C. Triplicate reactions were pooled and purified with the QIAquick 96 PCR Purification Kit (Qiagen). Sequencing reaction were preformed using 3 μl of PCR product, forward or reverse PCR primer, and BigDyeTerminator chemistry (Perkin-Elmer). Sequencing reactions were purified using Centri-Sep 96 plates (Princeton Separations). Electrophoresis and base calling was performed using a 3700 DNA Analyzer (Perkin-Elmer). Sequence data for the PCR fragments were built into contiguous mtDNA sequences using extensive source code modifications in the Contig Assembly Program (CAP; Thompson, J. D. et al., (1994) Nucl Acids Res 22:4673-4680) and aligned with the published sequence of mitochondrial DNA (e.g., SEQ ID NO:1) using modified publicly available FASTA (Pearson et al., 1990 Proc. Nat. Acad. Sci. USA 85:2444) and CLUSTALW (Thompson et al., 1994 Nucl. Ac. Res. 22:4673) software that was modified in order to identify nucleotide substitutions. Data analysis included categorization of sample sequences according to various parameters, including: patient haplogroup, mtDNA gene region in which an identified SNP resided and, for protein encoding mtDNA genes in which a SNP was identified, whether the SNP was a synonymous substitution (i.e., resulted in no change in the amino acid sequence of the encoded protein) or a non-synonymous substitution (i.e., resulted in a different amino acid sequence for the encoded protein). Full length mtDNA sequences from 560 unrelated human subjects are set forth as SEQ ID NOS:2-561.

### Example 3

### Single Nucleotide Polymorphisms in Mirochondrial DNA that Segregate with Specific Haplogroups

The distribution of mtDNA haplogroups amongst the 435 individual samples of European origin were 52%, 10.8%, 9.7%,10.6%, 7.6%, 1.8%, 3.2%, 1.8% and 2.5% for the haplogroups H (n=226), K (n=47), U (n=42), T (n=46), J (n=33), W (n=8), I (n=14), V (n=8) and X (n=11) respectively. The African mtDNA haplogroups L1, L2, and L3 were represented by 3.3%, 4.1%, and 7.4%, respectively, in our population before collection of additional African American individuals in order to expand data sets for the L haplogroups, which totaled 56 individual subjects; 69 samples were obtained from individual subjects of Asian descent. Novel polymorphisms were identified that were associated, either as “haplogroup-specific” or “haplogroup-associated” polymorphisms as described above, with one (haplogroup-specific) or more (haplogroup-associated) of mtDNA haplogroups A, B, C, D, E, H, I, J, K, L1, L2, L3, T, U, V, W and X (Tables 3 and 4). Novel nucleotide substitutions that were specific for individual European haplogroups are C114T, C497T, T1189C, A3480G, T9698C, A10550G, A10978C, T11299C, A11470G, T12954C, C14167T and T14798C for haplogroup K; T3197C, A7768G, G9477A, T13617C, T14182, A14793G, A15218G and C16256T for haplogroup U; G228A, C295T, C462T and G15257A for haplogroup J; G930A, G1888A, T5426C, C6489A, G8697A, A11812G, T13965C, A14233G and C16296T for haplogroup T; T1243C, A3505G, G5046A, G5460A, C11674T and G15884C for haplogroup W; T250C, G12501A, and A13780G for haplogroup I; T239C, C456T, T4336C, T677C, A16162G for haplogroup H; T16298C for haplogroup V; G225A, T226C, G6371T, C8393T, A13966G, and G15927A for haplogroup X. Other novel SNPs are shared by two of the European haplogroups, i.e., A181 IG and Al 1467G, which occurred in haplogroups U and K; G207A in haplogroups I and W; T4216C, A11251G, and C15452A in haplogroups J and T; T16304C in haplogroups H and T; A15924G in haplogroups I and K; G13708A in haplogroups J and X. Many of these novel SNPs allowed for the identification of novel subgroups for haplotypes H, K, U, J, and T based on CLUSTALW/NJPLOT⁷ analysis (FIG. 1).

Nucleotide substitutions at positions T489C, G709A, G3010A, T6221C, G11914A, C12705T, G14905A, G15043A, C16223T, C16294T and T16362C were found in European and African haplogroups. In this regard, they are “haplogroup associated” rather than “haplogroup specific”. The finding that all individuals not belonging to haplogroup H carried the nucleotide substitutions A73G, C7028T, and G11719A, which were absent in most of the H haplotypes (93-99%), confirmed a previous report (Andrews, R. M. et al. (1999) Nature Genetics 23:147).

### Example 4

### Single Nucleotide Polymorphisms and Deletions in Mitochondrial DNA that Segregate with African Haplogroups

Nucleotide changes that were novel and unique to all members of the three African mtDNA haplogroups are A8701G, T9540C, and T10873C. In addition to the unique changes previously identified for the L1 and L2 haplogroups at T10810C and G16390A, respectively, the substitutions G247A, T825A, G2758A, T2885C, G3666A, T7146, C8468T, C8655T, G10688A, C13506T, T13789C, T14178C, G14560A, and C16187T were found in all (or all but one) haplogroup L1 mtDNAs, whereas the substitutions T2416C, G8206A, A9221G, T10155C, T11944C, and G13590A were present in all (or all but one) haplogroup L2 mtDNAs. Changes at nucleotide positions 2758, 2768, 3308, and 16187, previously reported and not associated with any mtDNA haplogroup, were found in haplogroup L1 mtDNA (Table 3). Nucleotide substitutions that were found both in haplogroups L1 and L2 in addition to C3594T were C182T, G769A, G1018A, A4104G, C7256T, T7521C, and C13650T. A nucleotide substitution found in haplogroups L1 and L3 was A13105G, and a substitution found in L2 and L3 was G15301A.

The African L3 haplotypes were identified based on the absence of the HpaI restriction site at nucleotide position 3592 which corresponds to the absence of a C to T transition at nucleotide position 3594, and also the absence of substitutions at positions 182, 769, 1018, 4104, 7256, 7521, and 13650. Nucleotide changes that occurred in L3 mtDNA only were A249del, A289del, A290del, C2092T, T2352C, C4883T, C5178A, C6587T, C8650T, A9545G, A14152G, C14668T, T15670C, T15942C, C3450T, T3552A, A4715G, G5733A, T6221C, C7196A, G8584A, C9449T, A10086G, G10373A, C10400T, A10819G, A13263G, C13914A, T14212C, T14318C, T14783C, A15311G, A15487T, A15824G, G15930A, T15944del, T16124C, T16325C, and C16327T. Many of these nucleotide changes were clustered in subgroups of L3 (FIG. 2). All of the L3 haplotypes were characterized by the absence of polymorphisms that were present in one or both of the other African haplogroups, except for substitutions A13105 and G15301A, which were also present in some of L1 and all of L2 mtDNAs, respectively. Of the three African haplogroups identified to date, the L3 mtDNA haplogroup that was found in the population sample described herein appeared to be of most recent origin, based according to non-limiting theory on the observation that the overall level of sequence divergence was lower than for the L1 and L2 haplogroups.

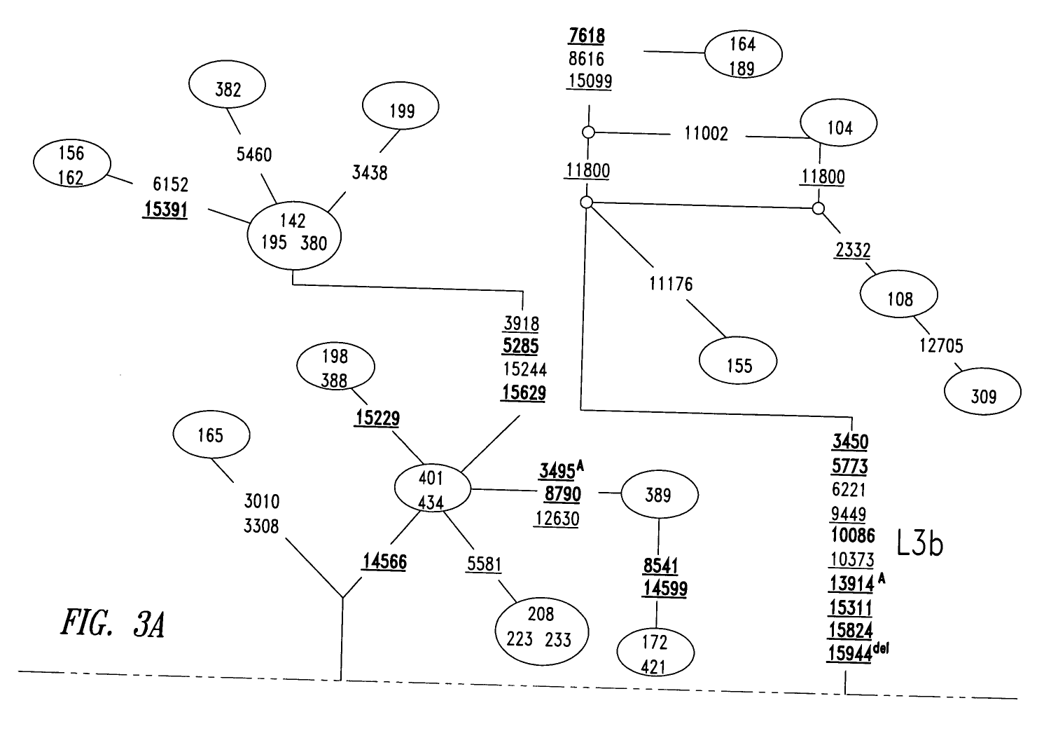

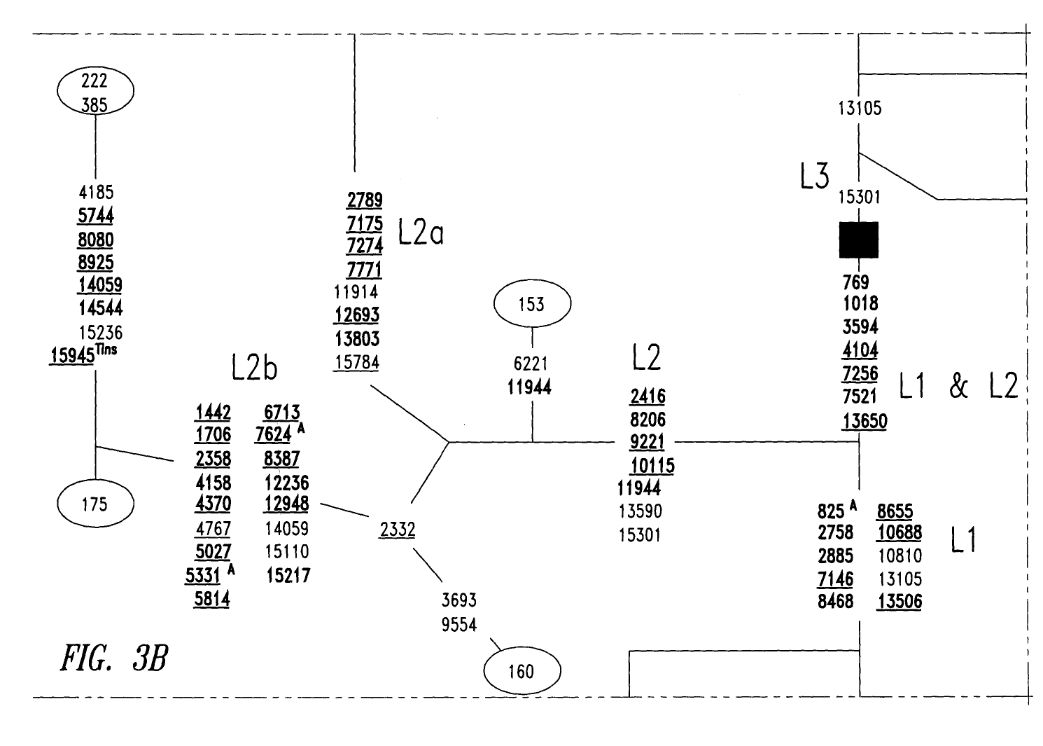

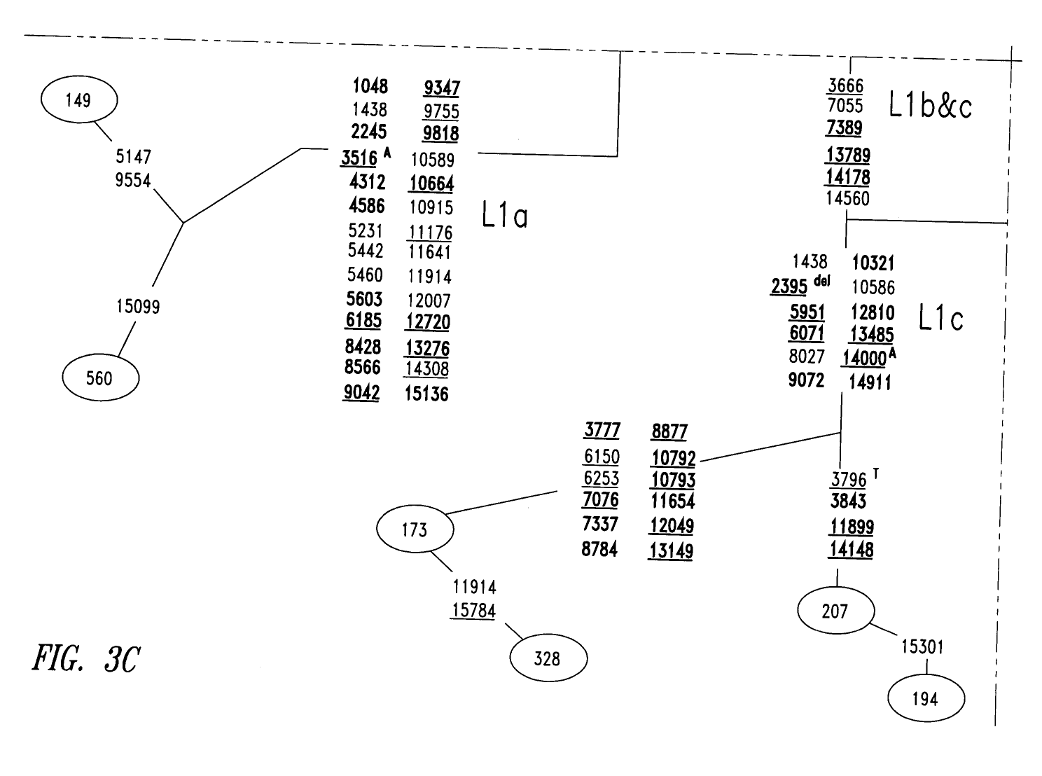

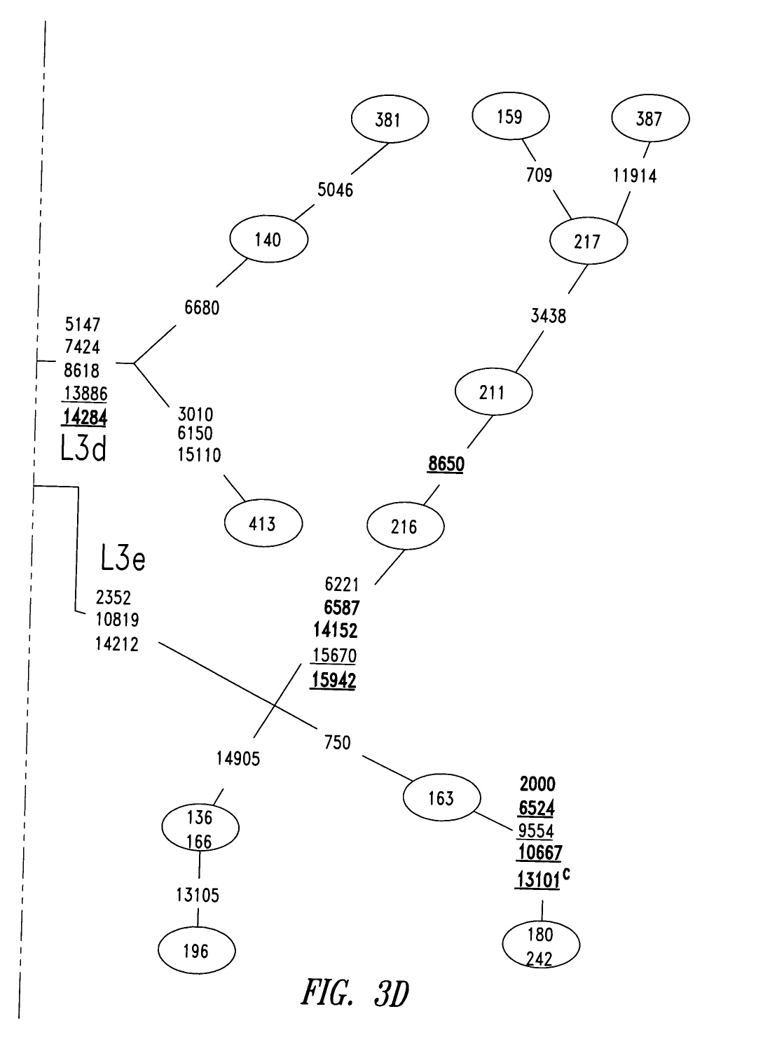

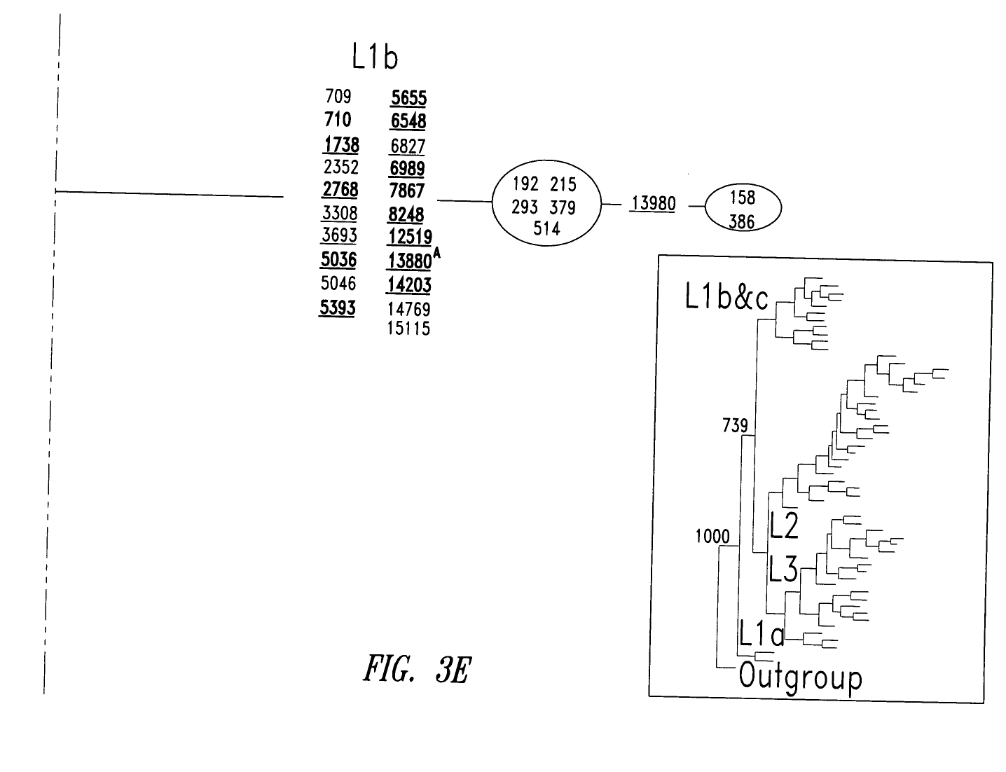

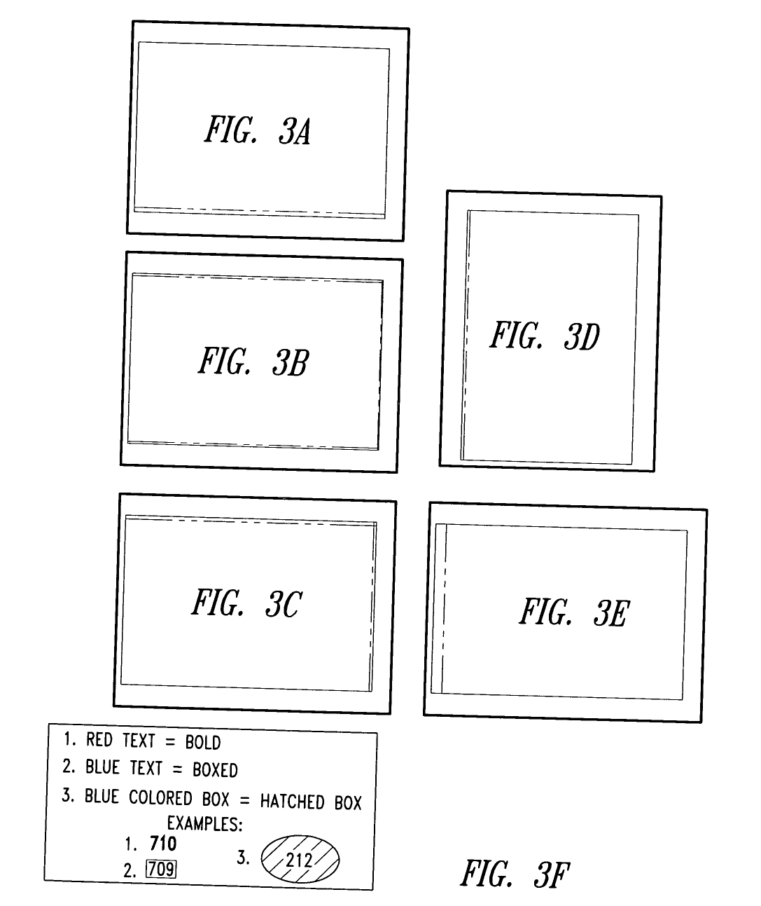

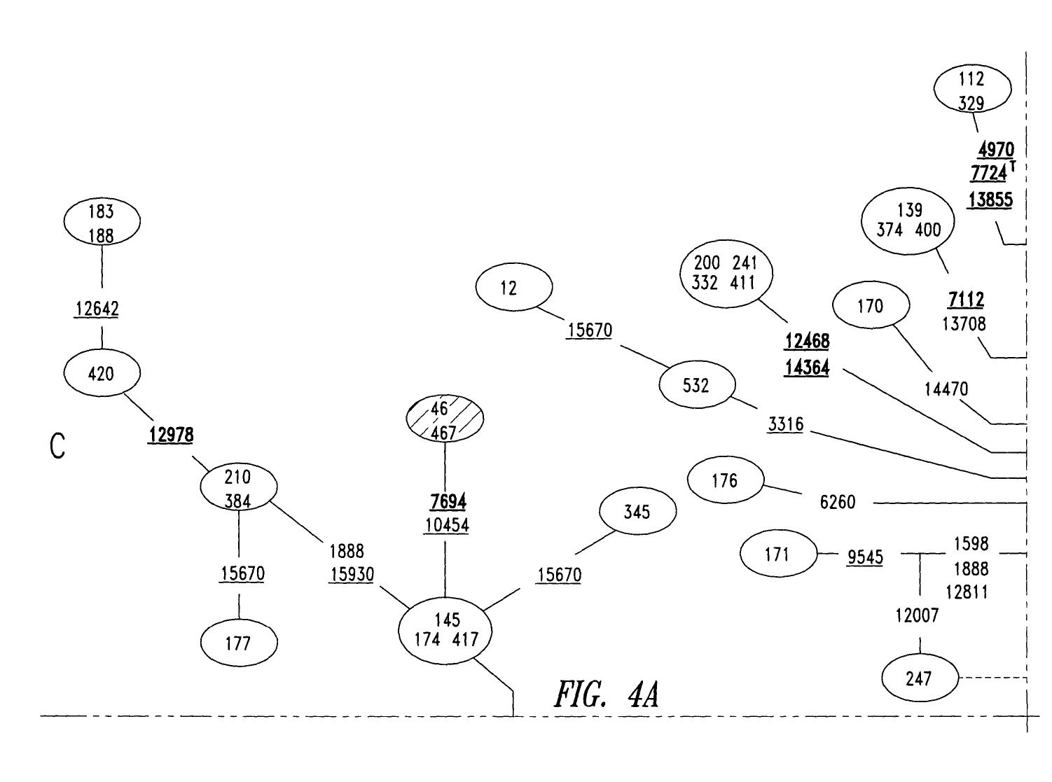

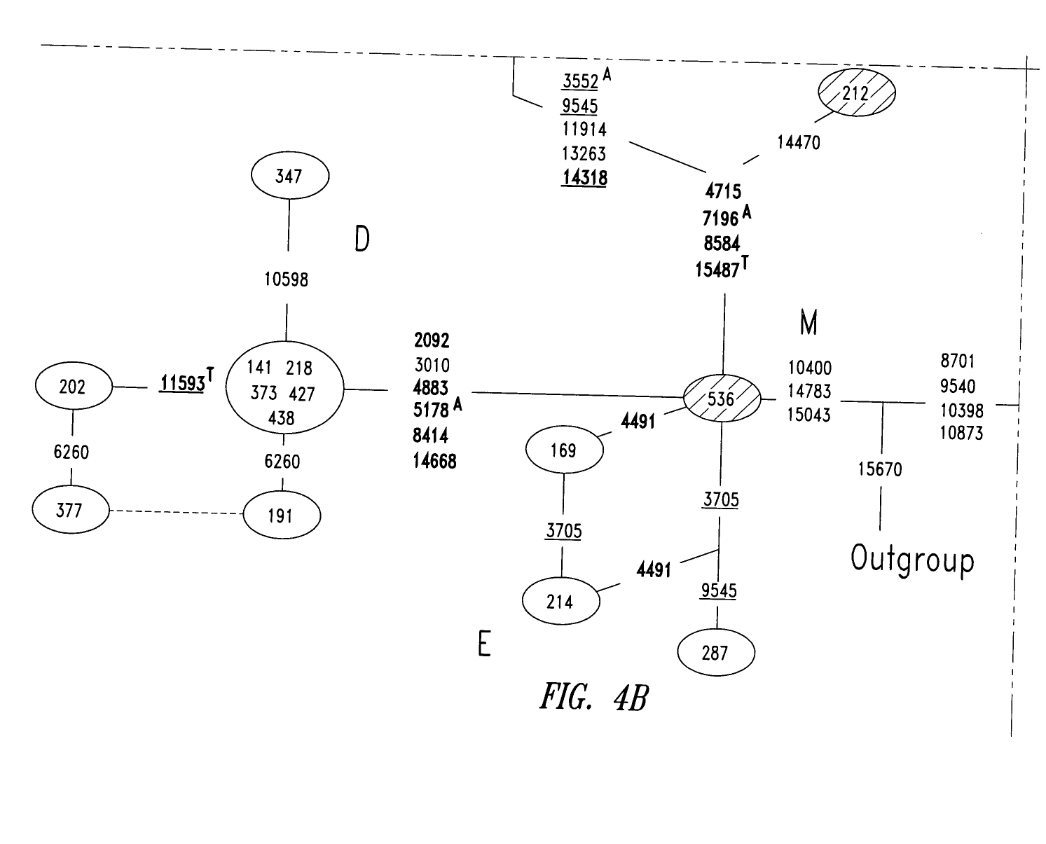

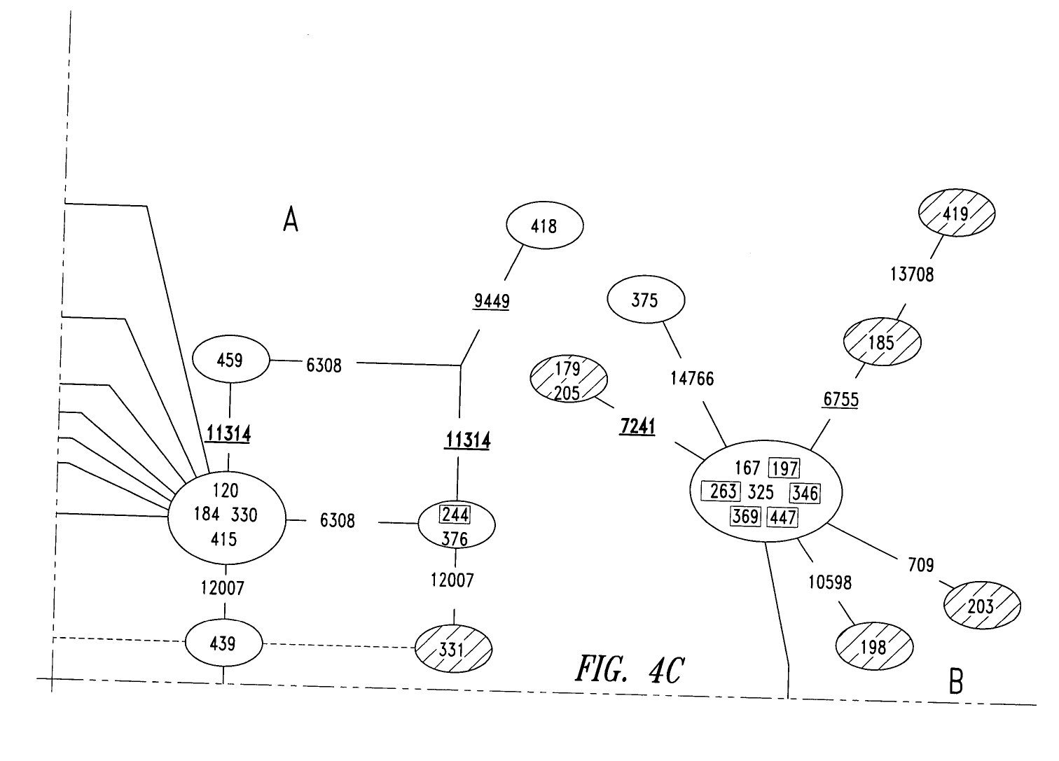

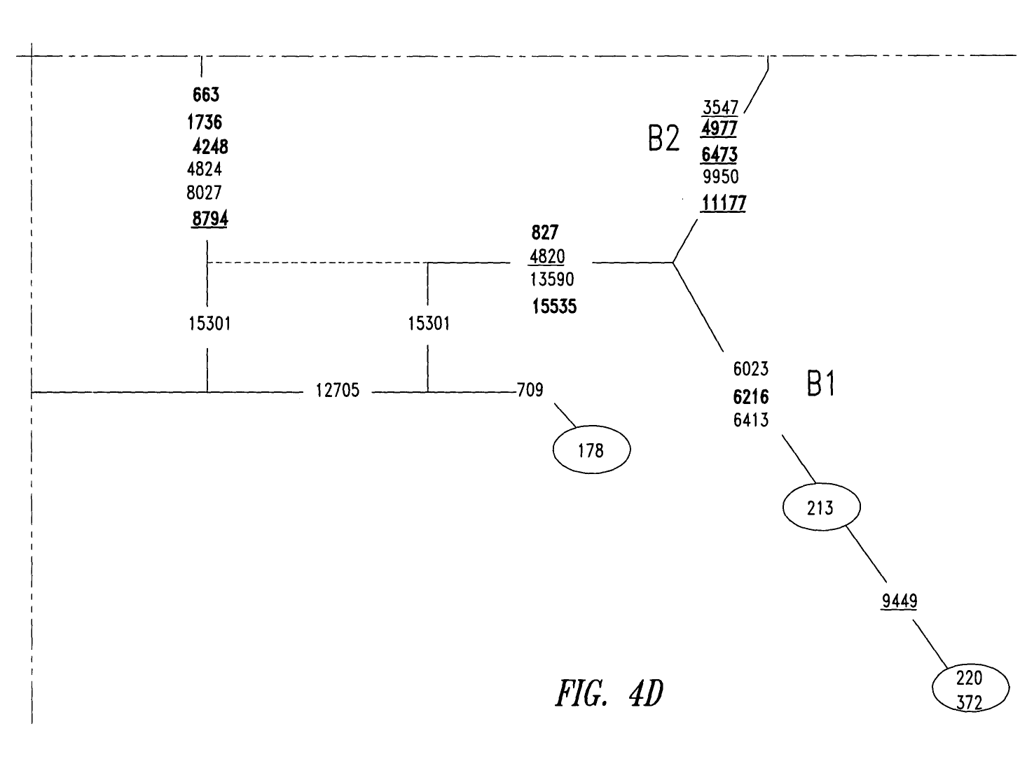

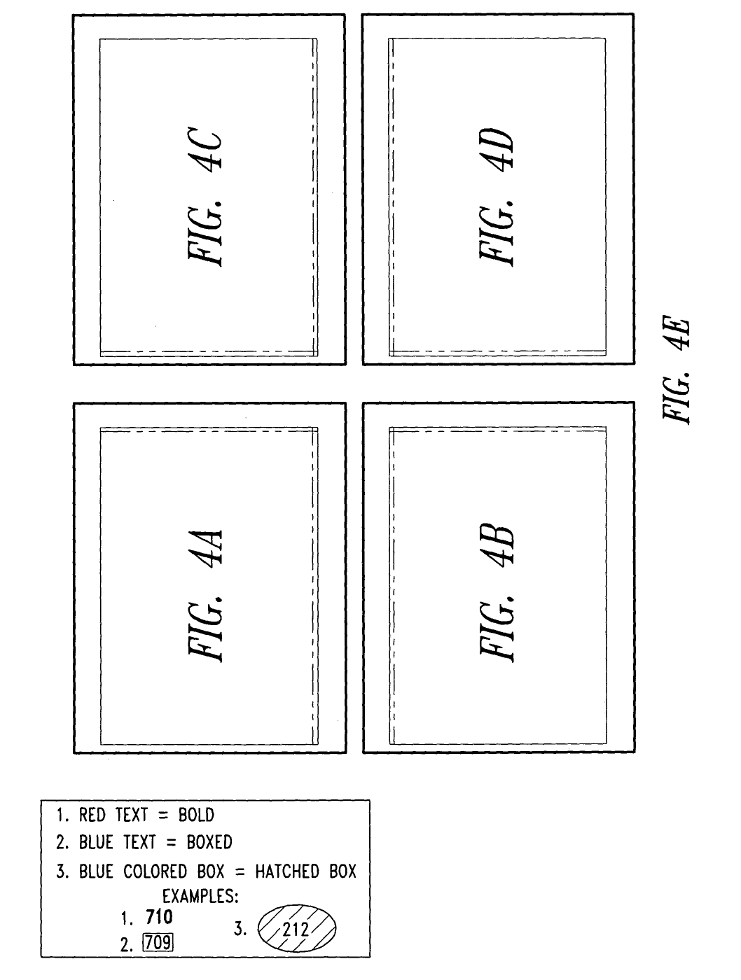

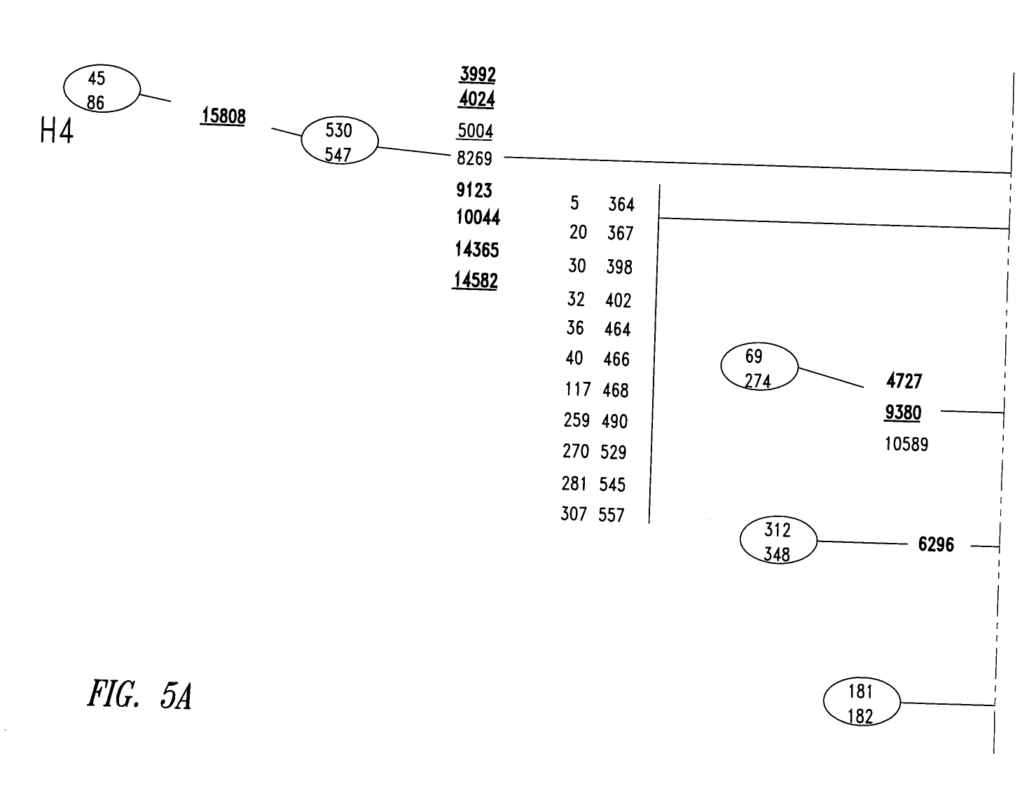

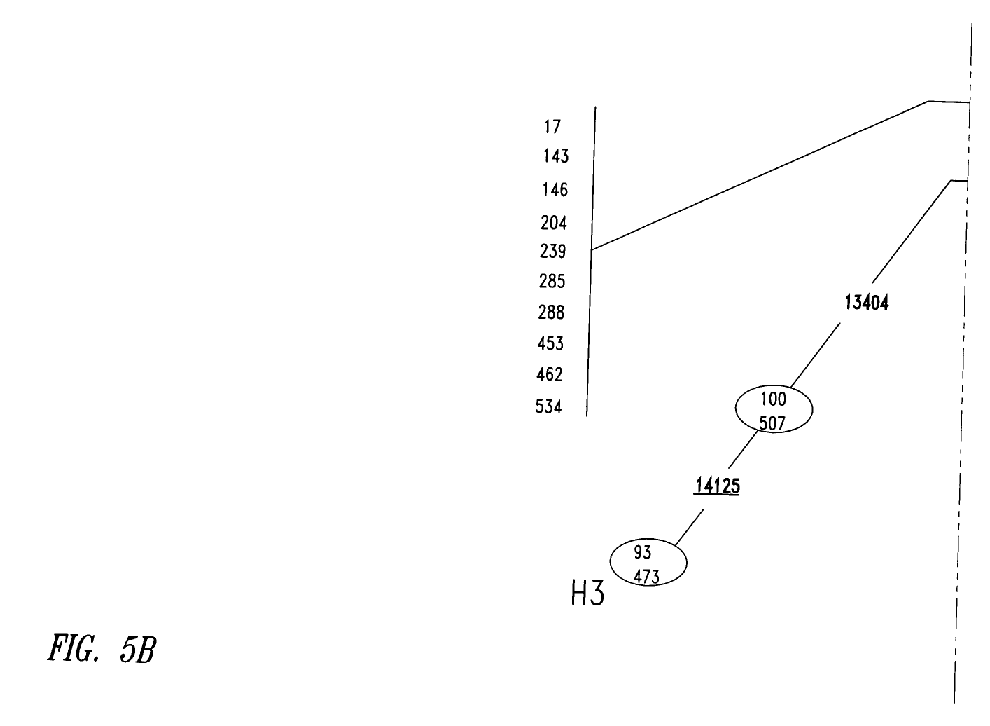

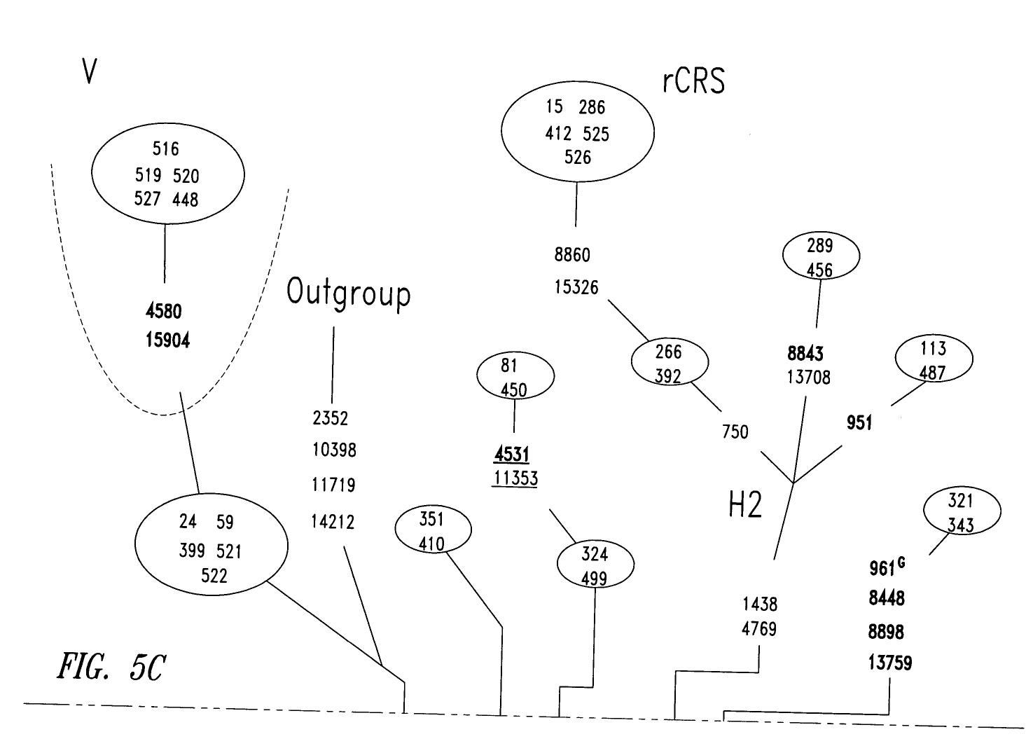

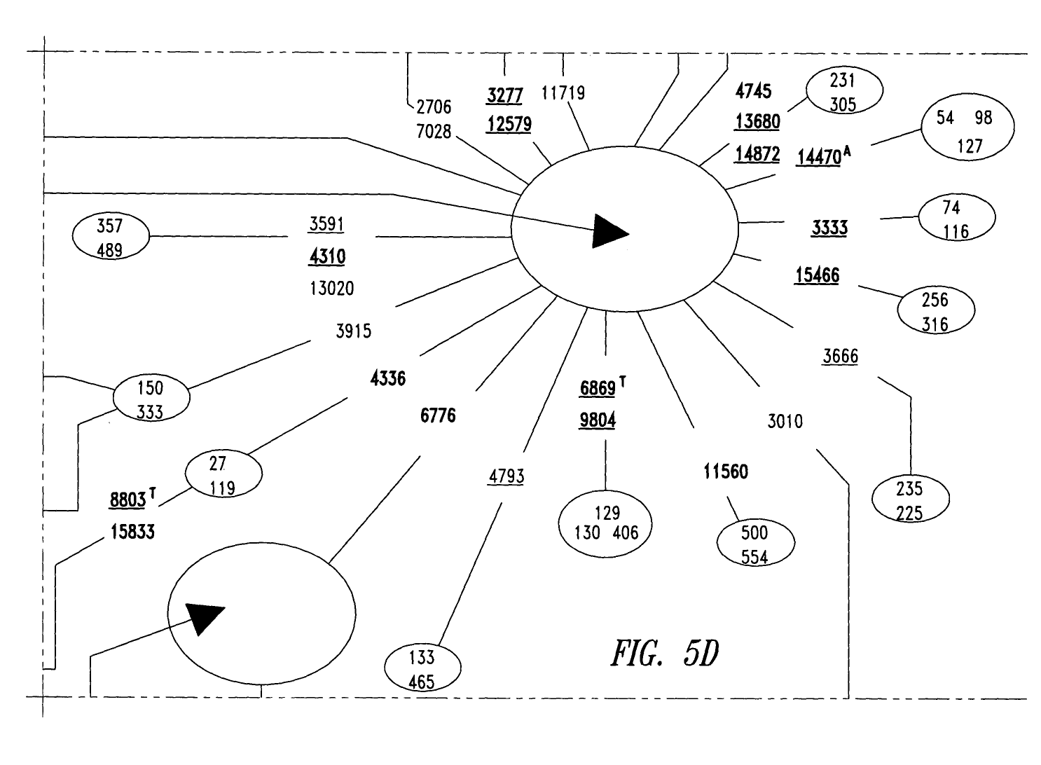

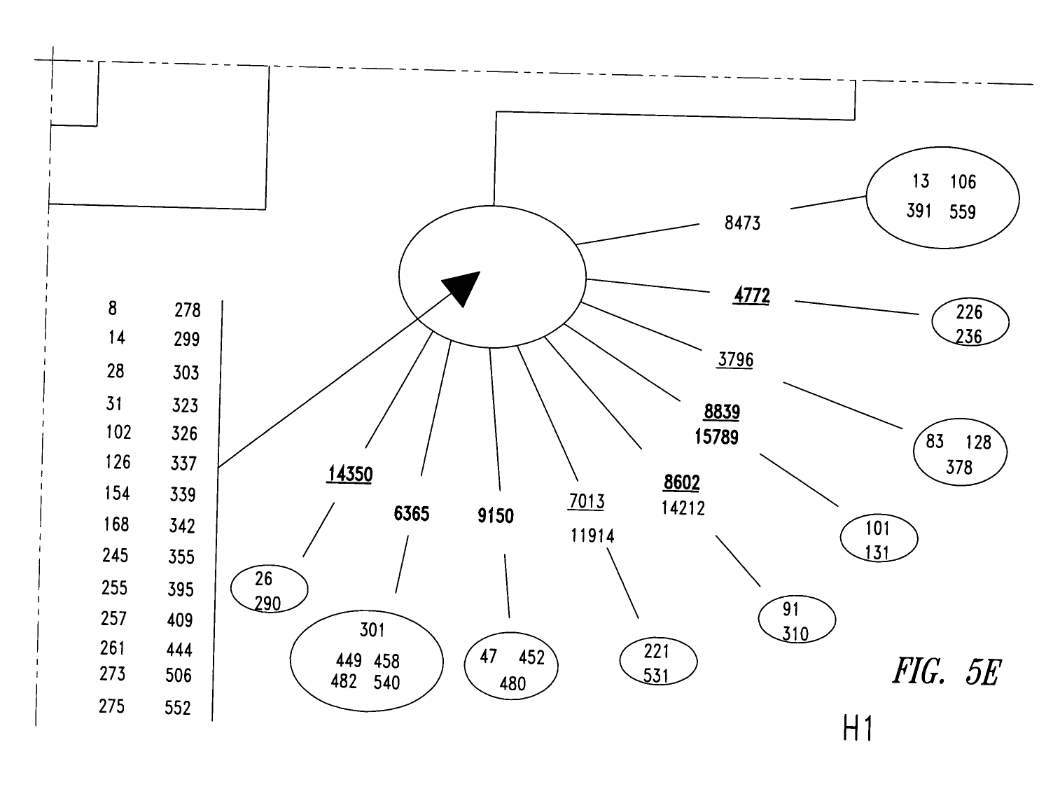

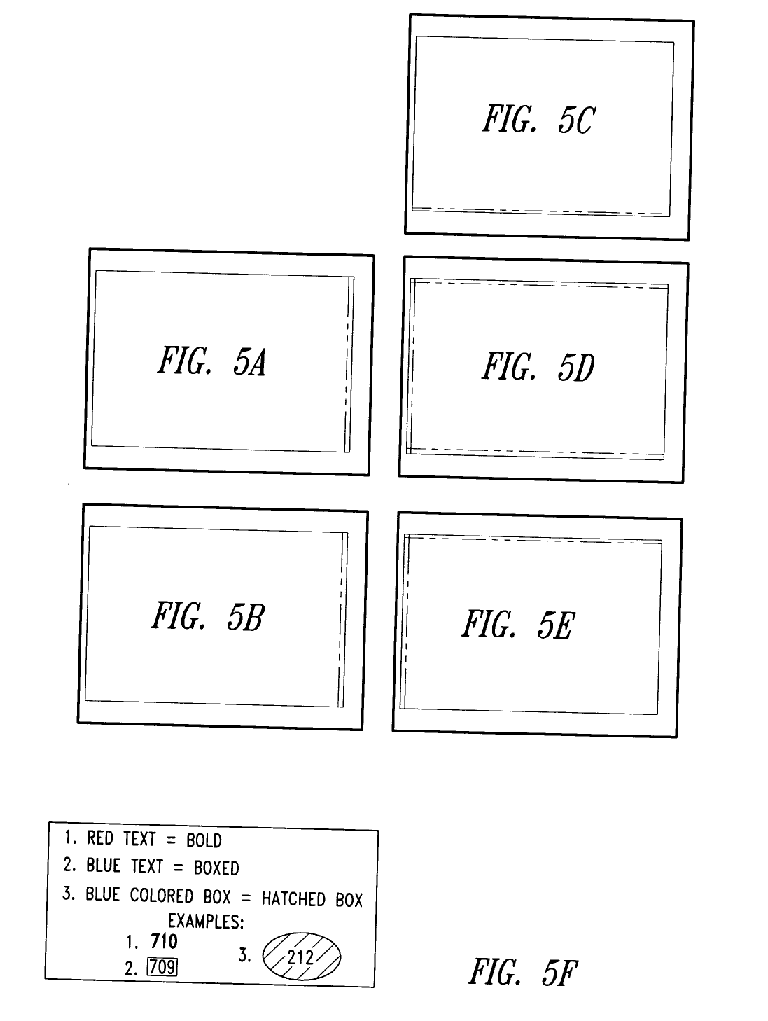

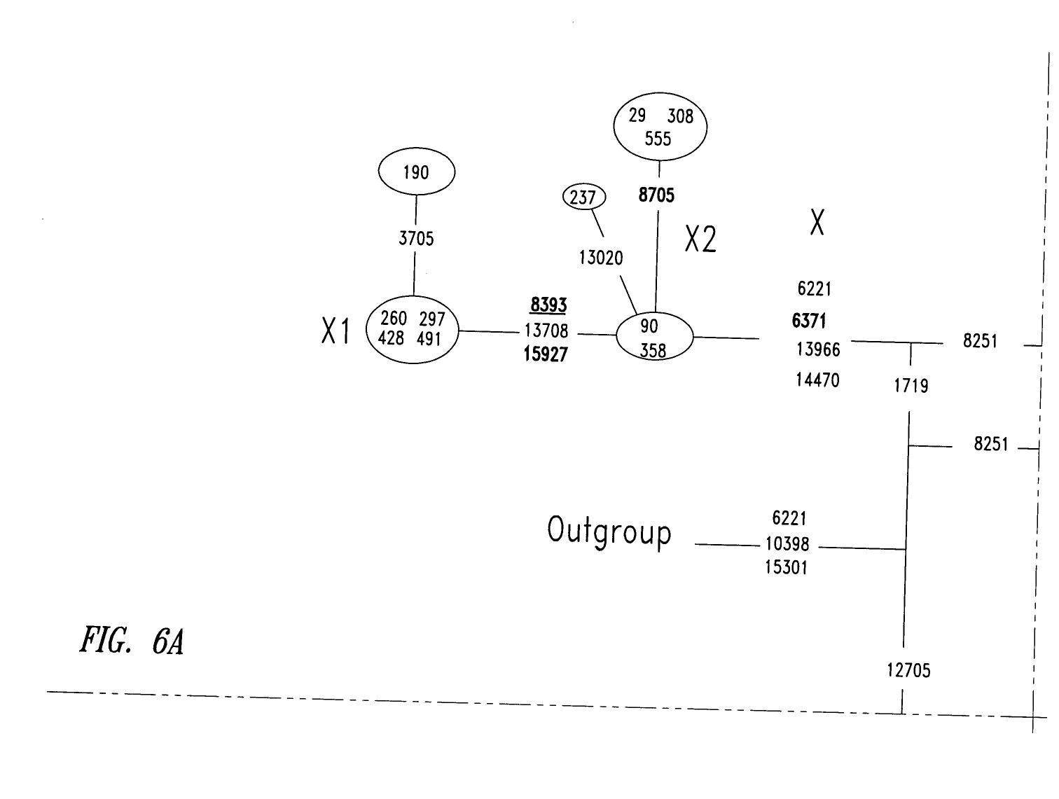

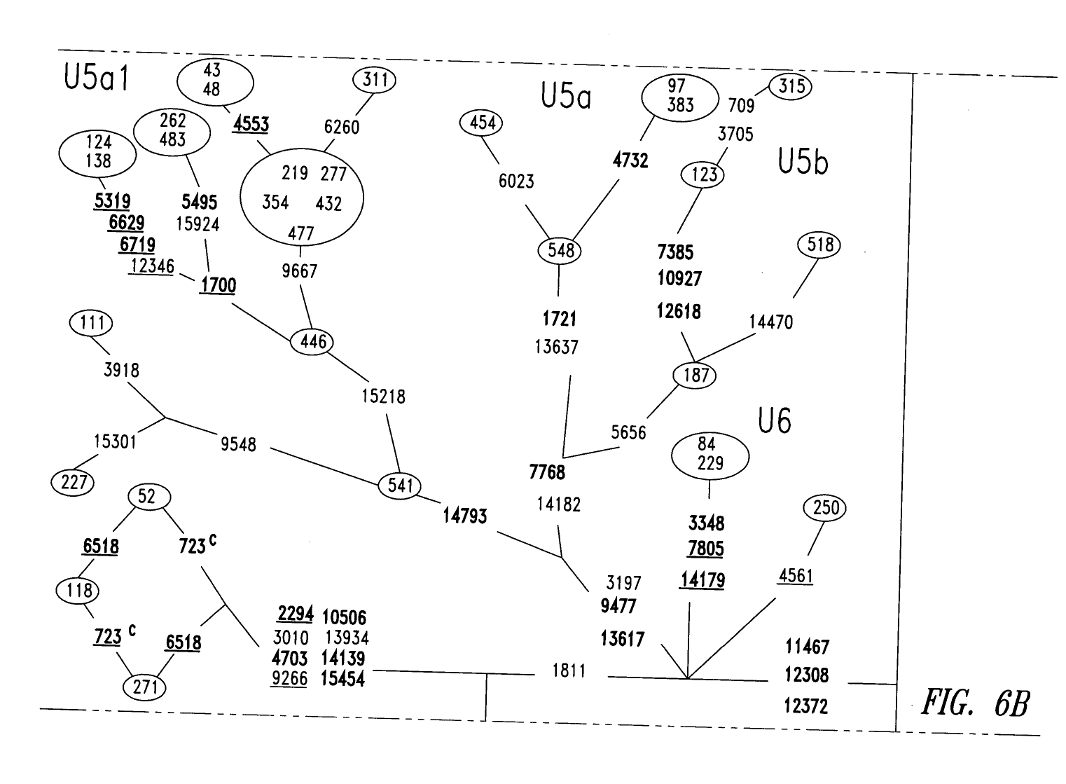

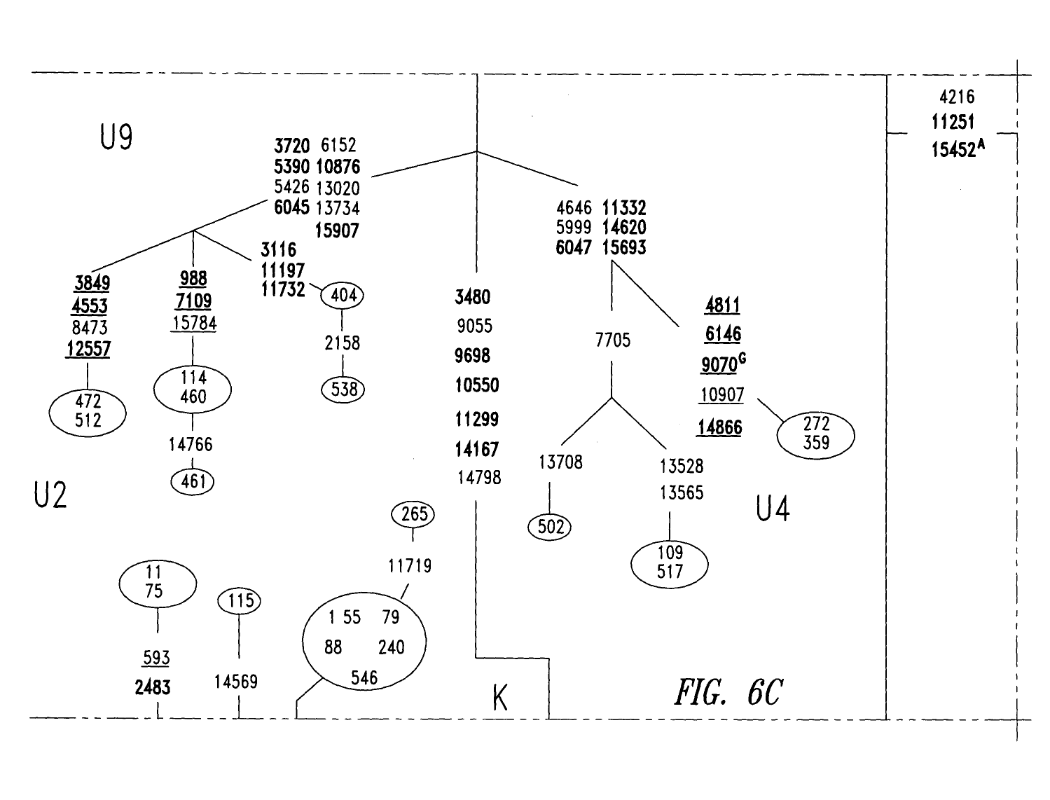

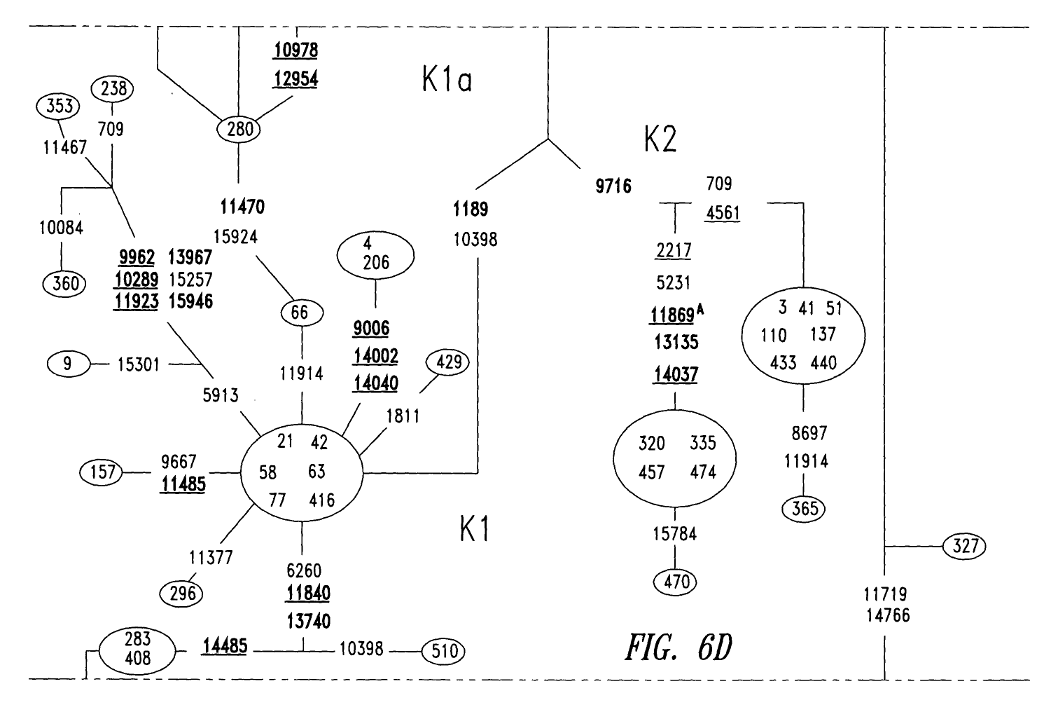

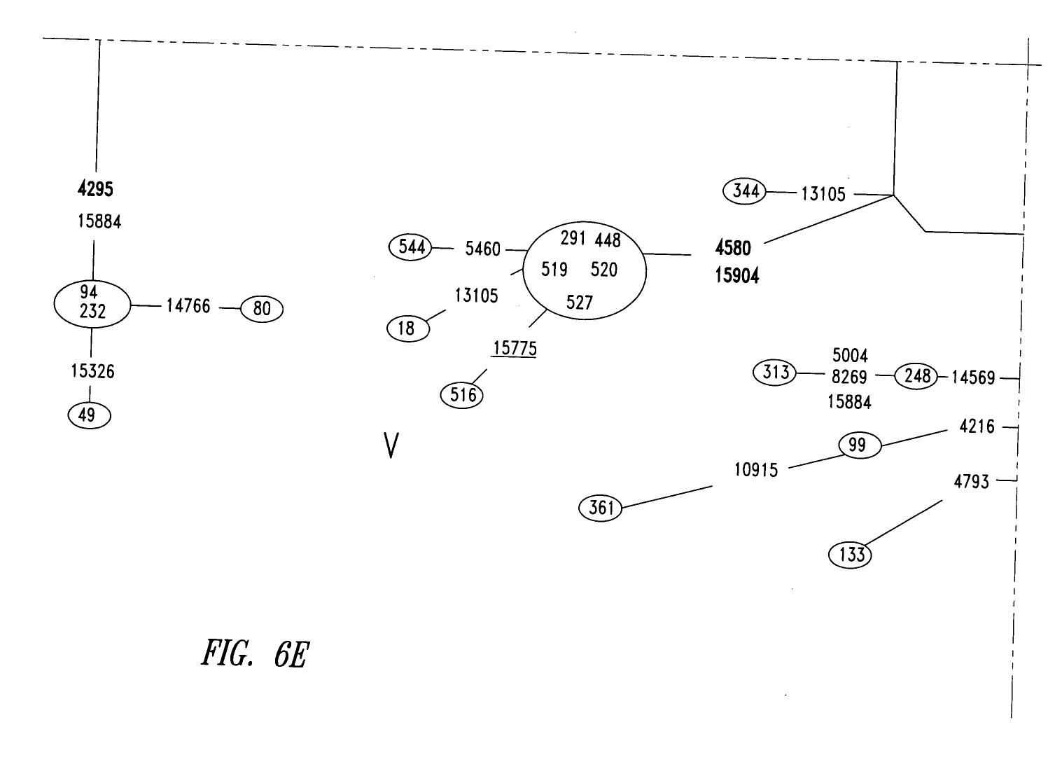

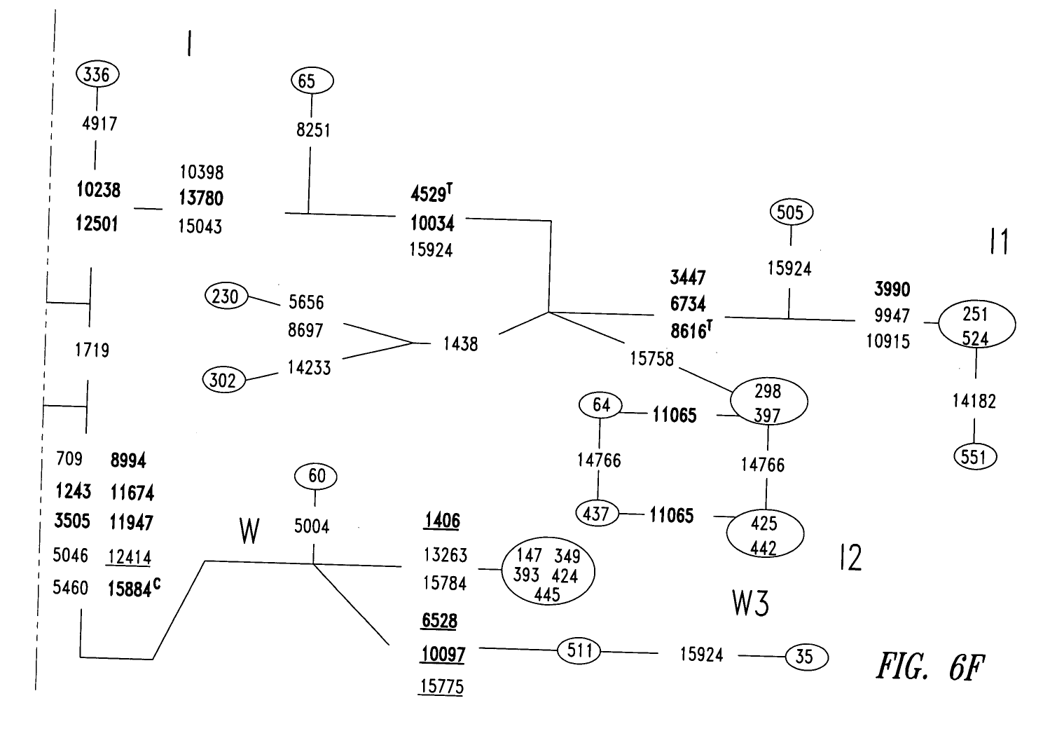

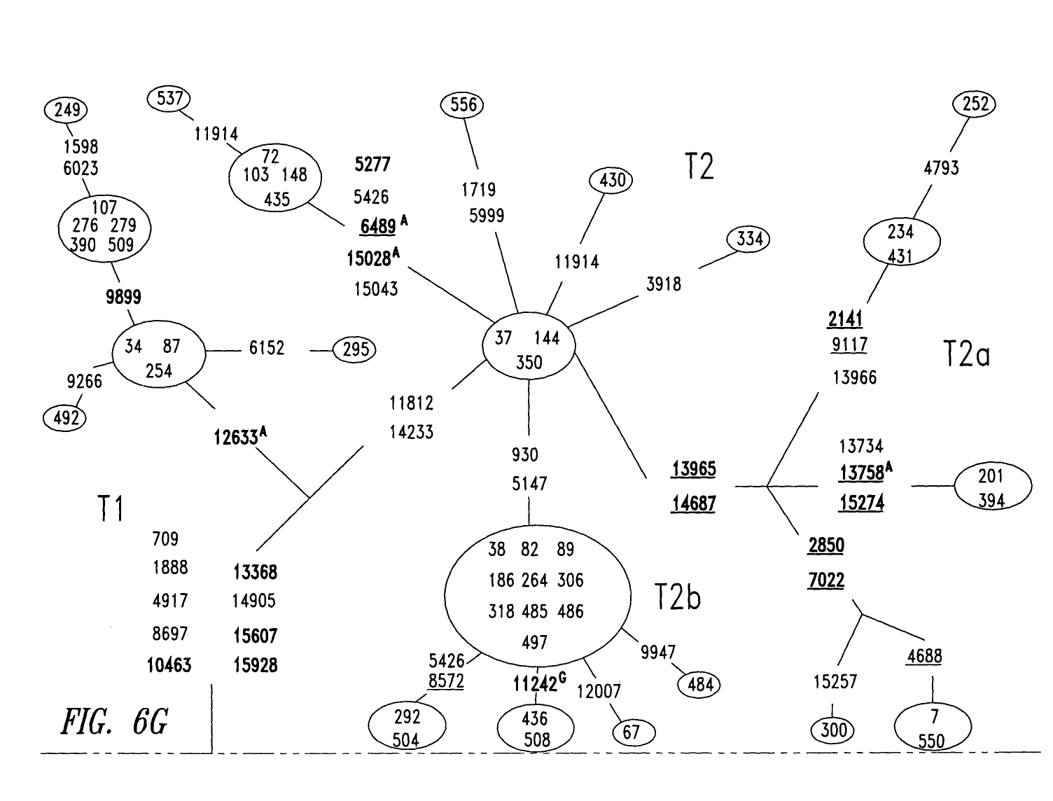

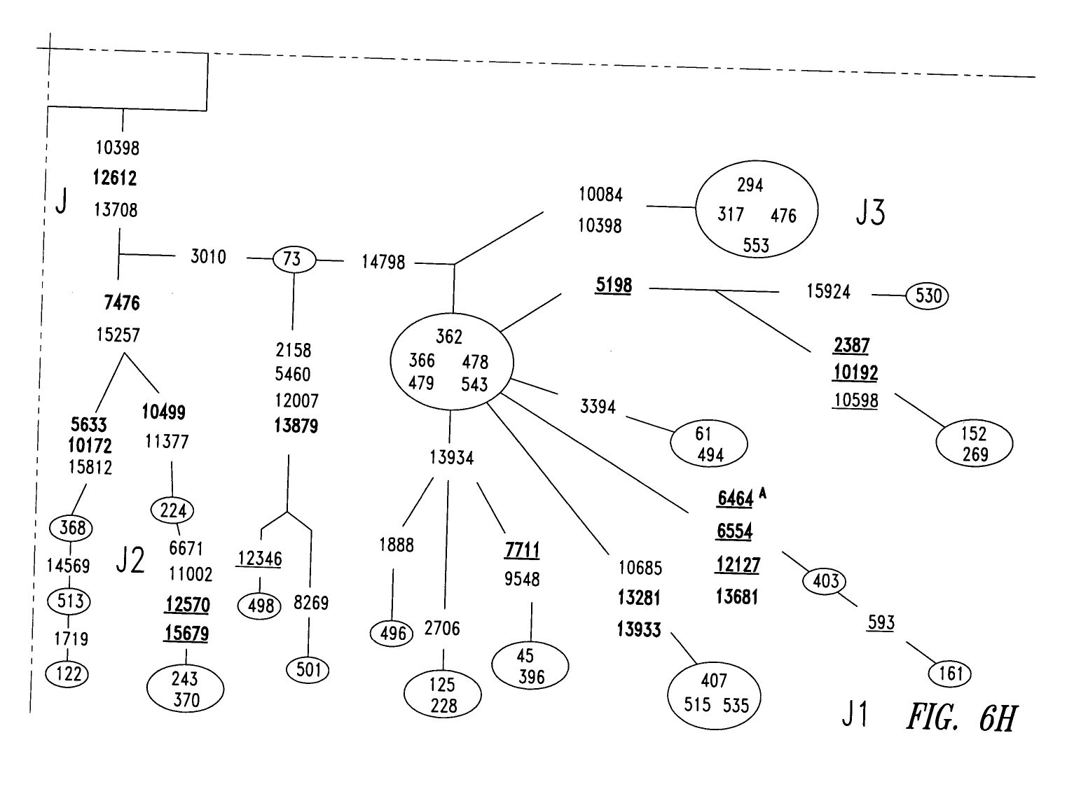

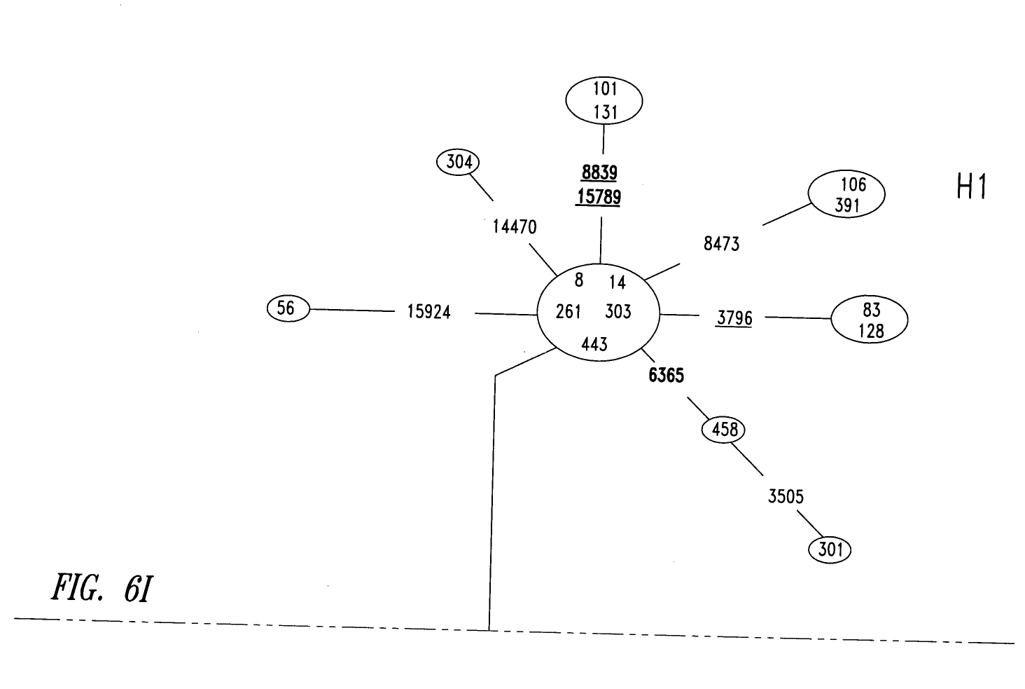

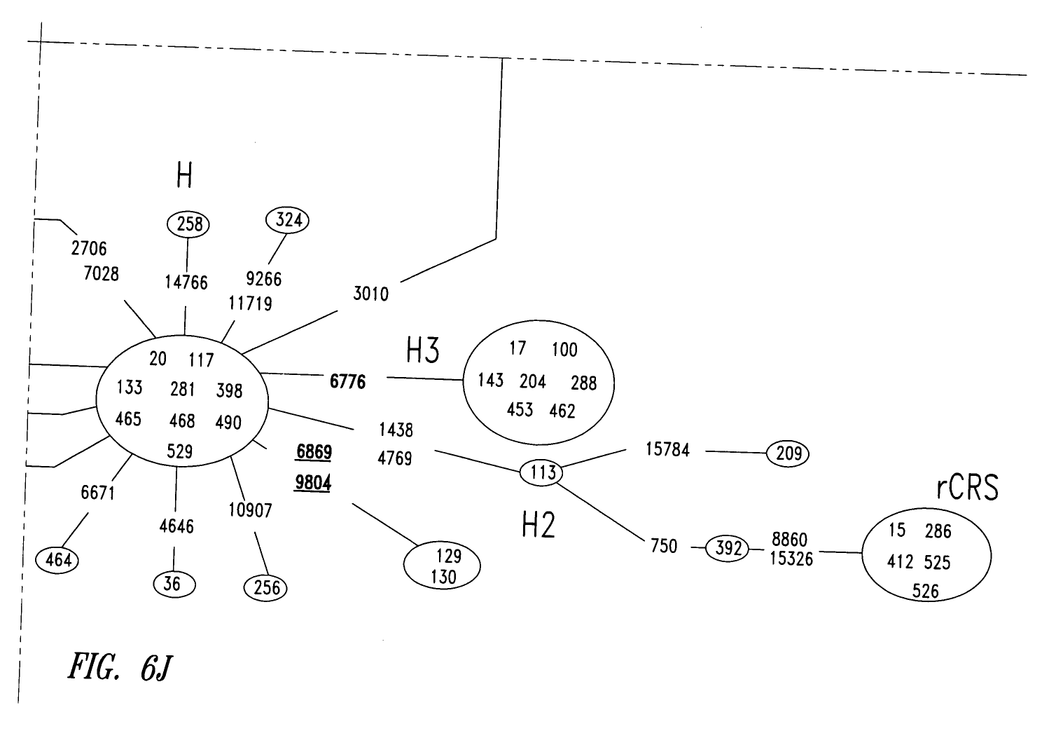

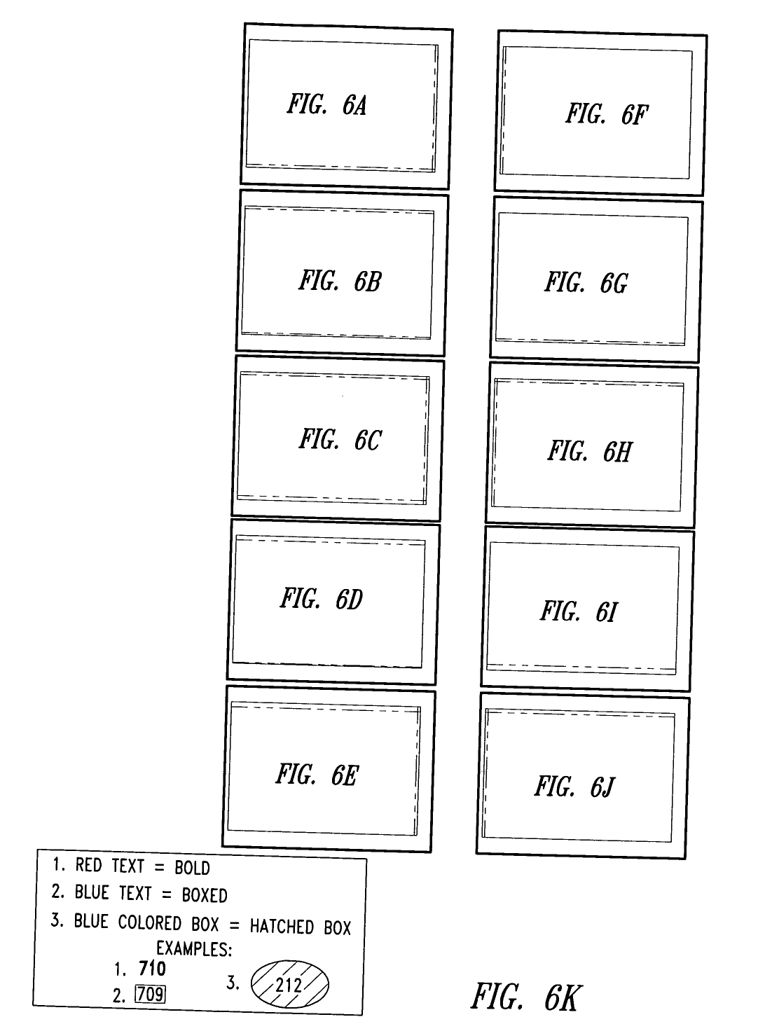

Additional clusters of polymorphisms (Tables 3 and 4) were detected and their relationships plotted using reduced meridian networks (Bandelt et al. 1995 Genetics 141:743) for European mtDNA haplogroups (FIG. 3), for European H and V mtDNA haplogroups (FIG. 4), for African mtDNA haplogroups (FIG. 5) and for Asian mtDNA mtDNA haplogroups (FIG. 6).

### Example 5

### Single Nucleotide Polymorphisms in Mitochondrial DNA that Correlate with Cancer and Tumors

None of the somatic changes previously reported in colorectal cancer tissue were found in tissues from the population analyzed here. More than one-third of the somatic changes previously found in lung, bladder, and head and neck tumors were also detected in the herein described population which included non-neoplastic blood, brain, and skeletal muscle samples (Table 2). The A1811G polymorphism was associated with mtDNA haplogroups K and U and was detected in 100% of the haplogroup K and in 29% of the haplogroup U mtDNAs for which the entire mtDNA was sequenced, and in an additional 24 blood samples from individuals for which only the ribosomal RNA genes were analyzed. The G2758A, A2768G, T3308C, G10688A, T10810C, and T16187C changes were associated specifically with haplogroup L1 mtDNAs and appeared together with a number of other polymorphisms (Table 1, FIG. 1). The A to C substitution (not C to A as previously reported) was found at nucleotide 16183 in 25 individual blood samples (12% of our sample population) and in four paired skeletal muscle samples from the same individuals. Furthermore, nucleotide substitutions at positions 150 (C to T), 195 (T to C) and 16519 (T to C) were common systemic polymorphisms which were found in 13%, 25% and 70%, respectively, of the herein characterized population. These substitutions were present in blood and brain, and in all skeletal muscle tissues paired with blood samples. Nucleotide substitution A4917G was found in all of haplogroup T mtDNAs where it was associated with other T-specific polymorphisms (Table 3, FIG. 1).

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for determining the mitochondrial haplogroup of a subject, comprising:
determining, in a biological sample comprising mitochondrial DNA from a subject, the presence of at least one mitochondrial single nucleotide polymorphism that is associated with a mitochondrial haplogroup.

2. The method of claim 1 wherein the mitochondrial DNA is amplified.

3. The method of claim 1 wherein at least one mitochondrial single nucleotide polymorphism that is associated with a mitochondrial haplogroup is present in a mitochondrial DNA region selected from the group consisting of a D-loop, a mitochondrial rRNA gene, a mitochondrial NADH dehydrogenase gene, a mitochondrial tRNA gene, a mitochondrial cytochrome c oxidase gene, a mitochondrial ATP synthase gene and a mitochondrial cytochrome b gene.

4. The method of claim 1 wherein at least one mitochondrial single nucleotide polymorphism that is associated with a mitochondrial haplogroup is selected from the group consisting of a haplogroup-specific polymorphism and a haplogroup-associated polymorphism, wherein a mitochondrial single nucleotide polymorphism that is haplogroup-specific is, for a halogroup selected from the group consisting of A, B, C, D, E, H, I, J, K, L1, L2, L3, T, U, V, W and X, located at a nucleotide that corresponds to a nucleotide position of SEQ ID NO:1 that is selected from the group consisting of:

| Nucleotide | | HAPLO- |
|---|---|---|
| Position | GENE | GROUP |
| 64 | D-LOOP | A |
| 235 | D-LOOP | A |
| 663 | 12S rRNA | A |
| 1598 | 12S rRNA | A |
| 1736 | 16S rRNA | A |
| 3316 | ND1 | A |
| 4248 | ND1 | A |
| 4824 | ND2 | A |
| 4970 | ND2 | A |
| 6308 | COI | A |
| 7112 | COI | A |
| 7724 | COII | A |
| 8794 | ATPase6 | A |
| 11314 | ND4 | A |
| 12468 | ND5 | A |
| 12811 | ND5 | A |
| 13855 | ND5 | A |
| 14364 | ND6 | A |
| 16111 | D-LOOP | A |
| 16290 | D-LOOP | A |
| 16319 | D-LOOP | A |
| 827 | 12S rRNA | B |
| 3547 | ND1 | B |
| 4820 | ND2 | B |
| 4977 | ND2 | B |
| 6023 | COI | B |
| 6216 | COI | B |
| 6413 | COI | B |
| 6473 | COI | B |
| 6755 | COI | B |
| 7241 | COI | B |
| 9950 | COIII | B |
| 11177 | ND4 | B |
| 15535 | CYT B | B |
| 16217 | D-LOOP | B |
| 249 | D-LOOP | C |
| 289 | D-LOOP | C |
| 290 | D-LOOP | C |
| 3552 | ND1 | C |
| 4715 | ND2 | C |
| 7196 | COI | C |
| 7694 | COII | C |
| 8584 | ATPase6 | C |
| 9545 | COIII | C |
| 10454 | tRNA-R | C |
| 12642 | ND5 | C |
| 12978 | ND5 | C |
| 14318 | ND6 | C |
| 15487 | CYT B | C |
| 15930 | tRNA-T | C |
| 2092 | 16S rRNA | D |
| 4883 | ND2 | D |
| 5178 | ND2 | D |
| 8414 | ATPase8 | D |
| 11593 | ND4 | D |
| 14668 | ND6 | D |
| 3027 | 16S rRNA | E |
| 3705 | ND1 | E |
| 4491 | ND2 | E |
| 7598 | COII | E |
| 239 | D-LOOP | H |
| 456 | D-LOOP | H |
| 477 | D-LOOP | H |
| 951 | 12S rRNA | H |
| 961 | 12S rRNA | H |
| 3277 | tRNA-L | H |
| 3333 | ND1 | H |
| 3591 | ND1 | H |
| 3796 | ND1 | H |
| 3915 | ND1 | H |
| 3992 | ND1 | H |
| 4024 | ND1 | H |
| 4310 | tRNA-l | H |
| 4336 | tRNA-Q | H |
| 4531 | ND2 | H |
| 4727 | ND2 | H |
| 4745 | ND2 | H |
| 4772 | ND2 | H |
| 4793 | ND2 | H |
| 5004 | ND2 | H |
| 6365 | COI | H |
| 6776 | COI | H |
| 6869 | COI | H |
| 7013 | COI | H |
| 8269 | COII | H |
| 8448 | ATPase8 | H |
| 8602 | ATPase6 | H |
| 8803 | ATPase6 | H |
| 8839 | ATPase6 | H |
| 8843 | ATPase6 | H |
| 8898 | ATPase6 | H |
| 9123 | ATPase6 | H |
| 9150 | ATPase6 | H |
| 9380 | COIII | H |
| 9804 | COIII | H |
| 10044 | tRNA-G | H |
| 11353 | ND4 | H |
| 11560 | ND4 | H |
| 12579 | ND5 | H |
| 13404 | ND5 | H |
| 13680 | ND5 | H |
| 13759 | ND5 | H |
| 14125 | ND5 | H |
| 14350 | ND6 | H |
| 14365 | ND6 | H |
| 14470 | ND6 | H |
| 14582 | ND6 | H |
| 14872 | CYT B | H |
| 15466 | CYT B | H |
| 15789 | CYT B | H |
| 15808 | CYT B | H |
| 15833 | CYT B | H |
| 16162 | D-LOOP | H |
| 16293 | D-LOOP | H |
| 199 | D-LOOP | I |
| 250 | D-LOOP | I |
| 3447 | ND1 | I |
| 3990 | ND1 | I |
| 4529 | ND2 | I |
| 6734 | COI | I |
| 8616 | ATPase6 | I |
| 9947 | COIII | I |
| 10034 | tRNA-G | I |
| 10238 | ND3 | I |
| 11065 | ND4 | I |
| 12501 | ND5 | I |
| 13780 | ND5 | I |
| 15758 | CYT B | I |
| 16391 | D-LOOP | I |
| 228 | D-LOOP | J |
| 295 | D-LOOP | J |
| 462 | D-LOOP | J |
| 2158 | 16S rRNA | J |
| 2387 | 16S rRNA | J |
| 3394 | ND1 | J |
| 5198 | ND2 | J |
| 5633 | tRNA-A | J |
| 6464 | COI | J |
| 6554 | COI | J |
| 6671 | COI | J |
| 7476 | tRNA-S | J |
| 7711 | COII | J |
| 10084 | ND3 | J |
| 10172 | ND3 | J |
| 10192 | ND3 | J |
| 10499 | ND4L | J |
| 10598 | ND4L | J |
| 10685 | ND4L | J |
| 11377 | ND4 | J |
| 12127 | ND4 | J |
| 12570 | ND5 | J |
| 12612 | ND5 | J |
| 13281 | ND5 | J |
| 13681 | ND5 | J |
| 13879 | ND5 | J |
| 13933 | ND5 | J |
| 14569 | ND6 | J |
| 15679 | CYT B | J |
| 15812 | CYT B | J |
| 16069 | D-LOOP | J |
| 16092 | D-LOOP | J |
| 16261 | D-LOOP | J |
| 114 | D-LOOP | K |
| 497 | D-LOOP | K |
| 593 | tRNA-F | K |
| 1189 | 12S rRNA | K |
| 2217 | 16S rRNA | K |
| 2483 | 16S rRNA | K |
| 3480 | ND1 | K |
| 4295 | tRNA-I | K |
| 4561 | ND2 | K |
| 5814 | tRNA-C | K |
| 6260 | COI | K |
| 9006 | ATPase6 | K |
| 9055 | ATPase6 | K |
| 9698 | COIII | K |
| 9716 | COIII | K |
| 9962 | COIII | K |
| 10289 | ND3 | K |
| 10550 | ND4L | K |
| 10978 | ND4 | K |
| 11299 | ND4 | K |
| 11470 | ND4 | K |
| 11485 | ND4 | K |
| 11840 | ND4 | K |
| 11869 | ND4 | K |
| 11923 | ND4 | K |
| 12954 | ND5 | K |
| 13135 | ND5 | K |
| 13740 | ND5 | K |
| 13967 | ND5 | K |
| 14002 | ND5 | K |
| 14037 | ND5 | K |
| 14040 | ND5 | K |
| 14167 | ND6 | K |
| 15884 | CYT B | K |
| 15946 | tRNA-T | K |
| 16224 | D-LOOP | K |
| 16234 | D-LOOP | K |
| 16463 | D-LOOP | K |
| 185 | D-LOOP | L1 |
| 186 | D-LOOP | L1 |
| 189 | D-LOOP | L1 |
| 236 | D-LOOP | L1 |
| 247 | D-LOOP | L1 |
| 297 | D-LOOP | L1 |
| 357 | D-LOOP | L1 |
| 710 | 12S rRNA | L1 |
| 825 | 12S rRNA | L1 |
| 1048 | 12S rRNA | L1 |
| 1738 | 16S rRNA | L1 |
| 2245 | 16S rRNA | L1 |
| 2395 | 16S rRNA | L1 |
| 2758 | 16S rRNA | L1 |
| 2768 | 16S rRNA | L1 |
| 2885 | 16S rRNA | L1 |
| 3308 | ND1 | L1 |
| 3516 | ND1 | L1 |
| 3666 | ND1 | L1 |
| 3693 | ND1 | L1 |
| 3777 | ND1 | L1 |
| 3796 | ND1 | L1 |
| 3843 | ND1 | L1 |
| 4312 | tRNA-I | L1 |
| 4586 | ND2 | L1 |
| 5036 | ND2 | L1 |
| 5393 | ND2 | L1 |
| 5442 | ND2 | L1 |
| 5603 | tRNA-A | L1 |
| 5655 | tRNA-A | L1 |
| 5913 | COI | L1 |
| 5951 | COI | L1 |
| 6071 | COI | L1 |
| 6150 | COI | L1 |
| 6185 | COI | L1 |
| 6253 | COI | L1 |
| 6548 | COI | L1 |
| 6827 | COI | L1 |
| 6989 | COI | L1 |
| 7055 | COI | L1 |
| 7076 | COI | L1 |
| 7146 | COI | L1 |
| 7337 | COI | L1 |
| 7389 | COI | L1 |
| 7867 | COII | L1 |
| 8248 | COII | L1 |
| 8428 | ATPase8 | L1 |
| 8655 | ATPase6 | L1 |
| 8784 | ATPase6 | L1 |
| 8877 | ATPase6 | L1 |
| 9042 | ATPase6 | L1 |
| 9072 | ATPase6 | L1 |
| 9347 | COIII | L1 |
| 9755 | COIII | L1 |
| 9818 | COIII | L1 |
| 10321 | ND3 | L1 |
| 10586 | ND4L | L1 |
| 10589 | ND4L | L1 |
| 10664 | ND4L | L1 |
| 10688 | ND4L | L1 |
| 10792 | ND4 | L1 |
| 10793 | ND4 | L1 |
| 10810 | ND4 | L1 |
| 11176 | ND4 | L1 |
| 11641 | ND4 | L1 |
| 11654 | ND4 | L1 |
| 11899 | ND4 | L1 |
| 12007 | ND4 | L1 |
| 12049 | ND4 | L1 |
| 12519 | ND5 | L1 |
| 12720 | ND5 | L1 |
| 12810 | ND5 | L1 |
| 13149 | ND5 | L1 |
| 13276 | ND5 | L1 |
| 13485 | ND5 | L1 |
| 13506 | ND5 | L1 |
| 13789 | ND5 | L1 |
| 13880 | ND5 | L1 |
| 13980 | ND5 | L1 |
| 14000 | ND5 | L1 |
| 14148 | ND5 | L1 |
| 14178 | ND6 | L1 |
| 14203 | ND6 | L1 |
| 14308 | ND6 | L1 |
| 14560 | ND6 | L1 |
| 14769 | CYT B | L1 |
| 14911 | CYT B | L1 |
| 15115 | CYT B | L1 |
| 15136 | CYT B | L1 |
| 16148 | D-LOOP | L1 |
| 16187 | D-LOOP | L1 |
| 16188 | D-LOOP | L1 |
| 16230 | D-LOOP | L1 |
| 16264 | D-LOOP | L1 |
| 16265 | D-LOOP | L1 |
| 16360 | D-LOOP | L1 |
| 16527 | D-LOOP | L1 |
| 143 | D-LOOP | L2 |
| 1442 | 12S rRNA | L2 |
| 1706 | 16S rRNA | L2 |
| 2332 | 16S rRNA | L2 |
| 2358 | 16S rRNA | L2 |
| 2416 | 16S rRNA | L2 |
| 2789 | 16S rRNA | L2 |
| 3495 | ND1 | L2 |
| 3918 | ND1 | L2 |
| 4158 | ND1 | L2 |
| 4185 | ND1 | L2 |
| 4370 | tRNA-Q | L2 |
| 4767 | ND2 | L2 |
| 5027 | ND2 | L2 |
| 5285 | ND2 | L2 |
| 5331 | ND2 | L2 |
| 5581 | tRNA-W | L2 |
| 5744 | NON-CODING | L2 |
| 6713 | COI | L2 |
| 7175 | COI | L2 |
| 7274 | COI | L2 |
| 7624 | COII | L2 |
| 7771 | COII | L2 |
| 8080 | COII | L2 |
| 8206 | COII | L2 |
| 8387 | ATPase8 | L2 |
| 8541 | ATPase8 | L2 |
| 8790 | ATPase6 | L2 |
| 8925 | ATPase6 | L2 |
| 9221 | COIII | L2 |
| 10115 | ND3 | L2 |
| 11944 | ND4 | L2 |
| 12236 | tRNA-S | L2 |
| 12630 | ND5 | L2 |
| 12693 | ND5 | L2 |
| 12948 | ND5 | L2 |
| 13803 | ND5 | L2 |
| 14059 | ND5 | L2 |
| 14544 | ND6 | L2 |
| 14566 | ND6 | L2 |
| 14599 | ND6 | L2 |
| 15110 | CYT B | L2 |
| 15217 | CYT B | L2 |
| 15229 | CYT B | L2 |
| 15236 | CYT B | L2 |
| 15244 | CYT B | L2 |
| 15391 | CYT B | L2 |
| 15629 | CYT B | L2 |
| 15945 | tRNA-T | L2 |
| 16114 | D-LOOP | L2 |
| 16213 | D-LOOP | L2 |
| 16309 | D-LOOP | L2 |
| 16390 | D-LOOP | L2 |
| 200 | D-LOOP | L3 |
| 2000 | 16S rRNA | L3 |
| 3438 | ND1 | L3 |
| 3450 | ND1 | L3 |
| 5773 | tRNA-C | L3 |
| 6524 | COI | L3 |
| 6587 | COI | L3 |
| 6680 | COI | L3 |
| 7424 | COI | L3 |
| 7618 | COII | L3 |
| 8616 | ATPase6 | L3 |
| 8618 | ATPase6 | L3 |
| 8650 | ATPase6 | L3 |
| 9554 | COIII | L3 |
| 10086 | ND3 | L3 |
| 10373 | ND3 | L3 |
| 10667 | ND4L | L3 |
| 10819 | ND4 | L3 |
| 11800 | ND4 | L3 |
| 13101 | ND5 | L3 |
| 13886 | ND5 | L3 |
| 13914 | ND5 | L3 |
| 14152 | ND6 | L3 |
| 14212 | ND6 | L3 |
| 14284 | ND6 | L3 |
| 15099 | CYT B | L3 |
| 15311 | CYT B | L3 |
| 15670 | CYT B | L3 |
| 15824 | CYT B | L3 |
| 15942 | tRNA-T | L3 |
| 15944 | tRNA-T | L3 |
| 16124 | D-LOOP | L3 |
| 16327 | D-LOOP | L3 |
| 930 | 12S rRNA | T |
| 2141 | 16S rRNA | T |
| 2850 | 16S rRNA | T |
| 4688 | ND2 | T |
| 4917 | ND2 | T |
| 5277 | ND2 | T |
| 6489 | COI | T |
| 7022 | COI | T |
| 8572 | ATPase6 | T |
| 8697 | ATPase6 | T |
| 9117 | ATPase6 | T |
| 9899 | COIII | T |
| 10463 | tRNA-R | T |
| 11242 | ND4 | T |
| 11812 | ND4 | T |
| 12633 | ND5 | T |
| 13368 | ND5 | T |
| 13758 | ND5 | T |
| 13965 | ND5 | T |
| 14233 | ND6 | T |
| 14687 | tRNA-E | T |
| 14905 | CYT B | T |
| 15028 | CYT B | T |
| 15274 | CYT B | T |
| 15607 | CYT B | T |
| 15928 | tRNA-T | T |
| 16163 | D-LOOP | T |
| 16182 | D-LOOP | T |
| 16186 | D-LOOP | T |
| 16294 | D-LOOP | T |
| 16296 | D-LOOP | T |
| 16324 | D-LOOP | T |
| 988 | 12S rRNA | U |
| 1700 | 16S rRNA | U |
| 1721 | 16S rRNA | U |
| 2294 | 16S rRNA | U |
| 3116 | 16S rRNA | U |
| 3197 | 16S rRNA | U |
| 3348 | ND1 | U |
| 3720 | ND1 | U |
| 3849 | ND1 | U |
| 4553 | ND2 | U |
| 4646 | ND2 | U |
| 4703 | ND2 | U |
| 4732 | ND2 | U |
| 4811 | ND2 | U |
| 5319 | ND2 | U |
| 5390 | ND2 | U |
| 5495 | ND2 | U |
| 5656 | NON-CODING | U |
| 5999 | COI | U |
| 6045 | COI | U |
| 6047 | COI | U |
| 6146 | COI | U |
| 6518 | COI | U |
| 6629 | COI | U |
| 6719 | COI | U |
| 7109 | COI | U |
| 7385 | COI | U |
| 7768 | COII | U |
| 7805 | COII | U |
| 8473 | ATPase8 | U |
| 9070 | ATPase6 | U |
| 9266 | COIII | U |
| 9477 | COIII | U |
| 9667 | COIII | U |
| 10506 | ND4L | U |
| 10876 | ND4 | U |
| 10907 | ND4 | U |
| 10927 | ND4 | U |
| 11197 | ND4 | U |
| 11332 | ND4 | U |
| 11732 | ND4 | U |
| 12346 | ND5 | U |
| 12557 | ND5 | U |
| 12618 | ND5 | U |
| 13020 | ND5 | U |
| 13617 | ND5 | U |
| 13637 | ND5 | U |
| 14139 | ND5 | U |
| 14179 | ND6 | U |
| 14182 | ND6 | U |
| 14620 | ND6 | U |
| 14793 | CYT B | U |
| 14866 | CYT B | U |
| 15191 | CYT B | U |
| 15218 | CYT B | U |
| 15454 | CYT B | U |
| 15693 | CYT B | U |
| 15907 | tRNA-T | U |
| 16051 | D-LOOP | U |
| 16256 | D-LOOP | U |
| 16270 | D-LOOP | U |
| 16399 | D-LOOP | U |
| 4580 | ND2 | V |
| 15904 | tRNA-T | V |
| 194 | D-LOOP | W |
| 1243 | 12S rRNA | W |
| 1406 | 12S rRNA | W |
| 3505 | ND1 | W |
| 6528 | COI | W |
| 8994 | ATPase6 | W |
| 10097 | ND3 | W |
| 11674 | ND4 | W |
| 11947 | ND4 | W |
| 12414 | ND5 | W |
| 13263 | ND5 | W |
| 15775 | CYT B | W |
| 15884 | CYT B | W |
| 16292 | D-LOOP | W |
| 225 | D-LOOP | X |
| 226 | D-LOOP | X |
| 6371 | COI | X |
| 8393 | ATPase8 | X |
| 8705 | ATPase6 | X |
| 14470 | ND6 | X |
| 15927 | tRNA-T | X |
| −73 | D-LOOP | not present in [H] |
| −7028 | COI | not present in [H] |
| −11698 | ND4 | not present in [H] |
| and | CYT B | not present in [H] |
| 14766, | | |

and wherein a mitochondrial single nucleotide polymorphism that is haplogroup-associated is, for a haplogroup selected from the group consisting of A, B, C, D, E, H, I, J, K, L1, L2, L3, T, U, W and X, located at a nucleotide that corresponds to a nucleotide position of SEQ ID NO:1 that is selected from the group consisting of:

| Nucleotide | | |
|---|---|---|
| Position | Gene | Haplogroup |
| 16362 | D-LOOP | A, C, D, E, H, L3 |
| 12705 | ND5 | A, C, D, E, I, L1, L2, L3, W, X |
| 1888 | 16S rRNA | A, C, T |
| 13708 | ND5 | A, J, X |
| 8027 | COII | A, L1 |
| 153 | D-LOOP | A, X |
| 207 | D-LOOP | B, I, L2, W |
| 13590 | ND5 | B, L2 |
| 9449 | COIII | B, L3 |
| 499 | D-LOOP | B, U |
| 16325 | D-LOOP | C, D |
| 10400 | ND3 | C, D, E |
| 14783 | CYT B | C, D, E |
| 10398 | ND3 | C, D, E, I, J, K, L1, L2, L3 |
| 15043 | CYT B | C, D, E, I, T |
| 489 | D-LOOP | C, D, E, J |
| 8701 | ATPase6 | C, D, E, L1, L2, L3 |
| 9540 | COIII | C, D, E, L1, L2, L3 |
| 10873 | ND4 | C, D, E, L1, L2, L3 |
| 15301 | CYT B | C, D, E, L2, L3 |
| 11914 | ND4 | C, K, L1, L2 |
| 16298 | D-LOOP | C, V |
| 3010 | 16S rRNA | D, H, J, U |
| 16311 | D-LOOP | H, K, L1, L3 |
| 93 | D-LOOP | H, L1 |
| 16304 | D-LOOP | H, T |
| 16291 | D-LOOP | H, U |
| 16356 | D-LOOP | H, U |
| 16482 | D-LOOP | H, U |
| 15924 | tRNA-T | I, K, U |
| 10915 | ND4 | I, L1 |
| 204 | D-LOOP | I, L2, W |
| 8251 | COII | I, W |
| 1719 | 16S rRNA | I, X |
| 14798 | CYT B | J, K |
| 15257 | CYT B | J, K |
| 5460 | ND2 | J, L1, W |
| 185 | D-LOOP | J, L3 |
| 11002 | ND4 | J, L3 |
| 4216 | ND1 | J, T |
| 11251 | ND4 | J, T |
| 15452 | CYT B | J, T |
| 16126 | D-LOOP | J, T |
| 9548 | COIII | J, U |
| 13934 | ND5 | J, U |
| 5231 | ND2 | K, L1 |
| 709 | 12S rRNA | K, L1, T, W |
| 1811 | 16S rRNA | K, U |
| 11467 | ND4 | K, U |
| 12308 | tRNA-L | K, U |
| 12372 | ND5 | K, U |
| 182 | D-LOOP | L1, L2 |
| 198 | D-LOOP | L1, L2 |
| 769 | 12S rRNA | L1, L2 |
| 1018 | 12S rRNA | L1, L2 |
| 3594 | ND1 | L1, L2 |
| 4104 | ND1 | L1, L2 |
| 7256 | COI | L1, L2 |
| 7521 | tRNA-D | L1, L2 |
| 13650 | ND5 | L1, L2 |
| 16278 | D-LOOP | L1, L2, L3, X |
| 189 | D-LOOP | L1, L3 |
| 2352 | 16S rRNA | L1, L3 |
| 13105 | ND5 | L1, L3 |
| 5046 | ND2 | L1, W |
| 6152 | COI | L2, U |
| 15784 | CYT B | L2, U, W |
| 5147 | ND2 | L3, T |
| 6221 | COI | L3, X |
| 5426 | ND2 | T, U |
| 13734 | ND5 | T, U |
| and | ND5 | T, X |
| 13966. | | |

5. The method of claim 1 wherein the mitochondrial single nucleotide polymorphism that is associated with a mitochondrial haplogroup is a haplogroup-specific polymorphism that is present in members of only one haplogroup selected from the group consisting of A, B, C, D, E, H, I, J, K, L1, L2, L3, T, U, V, W and X, located at a nucleotide that corresponds to a nucleotide position of SEQ ID NO:1 that is selected from the group consisting of:

| Nucleotide | | HAPLO- |
|---|---|---|
| Position | GENE | GROUP |
| 64 | D-LOOP | A |
| 235 | D-LOOP | A |
| 663 | 12S rRNA | A |
| 1598 | 12S rRNA | A |
| 1736 | 16S rRNA | A |
| 3316 | ND1 | A |
| 4248 | ND1 | A |
| 4824 | ND2 | A |
| 4970 | ND2 | A |
| 6308 | COI | A |
| 7112 | COI | A |
| 7724 | COII | A |
| 8794 | ATPase6 | A |
| 11314 | ND4 | A |
| 12468 | ND5 | A |
| 12811 | ND5 | A |
| 13855 | ND5 | A |
| 14364 | ND6 | A |
| 16111 | D-LOOP | A |
| 16290 | D-LOOP | A |
| 16319 | D-LOOP | A |
| 827 | 12S rRNA | B |
| 3547 | ND1 | B |
| 4820 | ND2 | B |
| 4977 | ND2 | B |
| 6023 | COI | B |
| 6216 | COI | B |
| 6413 | COI | B |
| 6473 | COI | B |
| 6755 | COI | B |
| 7241 | COI | B |
| 9950 | COIII | B |
| 11177 | ND4 | B |
| 15535 | CYT B | B |
| 16217 | D-LOOP | B |
| 249 | D-LOOP | C |
| 289 | D-LOOP | C |
| 290 | D-LOOP | C |
| 3552 | ND1 | C |
| 4715 | ND2 | C |
| 7196 | COI | C |
| 7694 | COII | C |
| 8584 | ATPase6 | C |
| 9545 | COIII | C |
| 10454 | tRNA-R | C |
| 12642 | ND5 | C |
| 12978 | ND5 | C |
| 14318 | ND6 | C |
| 15487 | CYT B | C |
| 15930 | tRNA-T | C |
| 2092 | 16S rRNA | D |
| 4883 | ND2 | D |
| 5178 | ND2 | D |
| 8414 | ATPase8 | D |
| 11593 | ND4 | D |
| 14668 | ND6 | D |
| 3027 | 16S rRNA | E |
| 3705 | ND1 | E |
| 4491 | ND2 | E |
| 7598 | COII | E |
| 239 | D-LOOP | H |
| 456 | D-LOOP | H |
| 477 | D-LOOP | H |
| 951 | 12S rRNA | H |
| 961 | 12S rRNA | H |
| 3277 | tRNA-L | H |
| 3333 | ND1 | H |
| 3591 | ND1 | H |
| 3796 | ND1 | H |
| 3915 | ND1 | H |
| 3992 | ND1 | H |
| 4024 | ND1 | H |
| 4310 | tRNA-I | H |
| 4336 | tRNA-Q | H |
| 4531 | ND2 | H |
| 4727 | ND2 | H |
| 4745 | ND2 | H |
| 4772 | ND2 | H |
| 4793 | ND2 | H |
| 5004 | ND2 | H |
| 6365 | COI | H |
| 6776 | COI | H |
| 6869 | COI | H |
| 7013 | COI | H |
| 8269 | COII | H |
| 8448 | ATPase8 | H |
| 8602 | ATPase6 | H |
| 8803 | ATPase6 | H |
| 8839 | ATPase6 | H |
| 8843 | ATPase6 | H |
| 8898 | ATPase6 | H |
| 9123 | ATPase6 | H |
| 9150 | ATPase6 | H |
| 9380 | COIII | H |
| 9804 | COIII | H |
| 10044 | tRNA-G | H |
| 11353 | ND4 | H |
| 11560 | ND4 | H |
| 12579 | ND5 | H |
| 13404 | ND5 | H |
| 13680 | ND5 | H |
| 13759 | ND5 | H |
| 14125 | ND5 | H |
| 14350 | ND6 | H |
| 14365 | ND6 | H |
| 14470 | ND6 | H |
| 14582 | ND6 | H |
| 14872 | CYT B | H |
| 15466 | CYT B | H |
| 15789 | CYT B | H |
| 15808 | CYT B | H |
| 15833 | CYT B | H |
| 16162 | D-LOOP | H |
| 16293 | D-LOOP | H |
| 199 | D-LOOP | I |
| 250 | D-LOOP | I |
| 3447 | ND1 | I |
| 3990 | ND1 | I |
| 4529 | ND2 | I |
| 6734 | COI | I |
| 8616 | ATPase6 | I |
| 9947 | COIII | I |
| 10034 | tRNA-G | I |
| 10238 | ND3 | I |
| 11065 | ND4 | I |
| 12501 | ND5 | I |
| 13780 | ND5 | I |
| 15758 | CYT B | I |
| 16391 | D-LOOP | I |
| 228 | D-LOOP | J |
| 295 | D-LOOP | J |
| 462 | D-LOOP | J |
| 2158 | 16S rRNA | J |
| 2387 | 16S rRNA | J |
| 3394 | ND1 | J |
| 5198 | ND2 | J |
| 5633 | tRNA-A | J |
| 6464 | COI | J |
| 6554 | COI | J |
| 6671 | COI | J |
| 7476 | tRNA-S | J |
| 7711 | COII | J |
| 10084 | ND3 | J |
| 10172 | ND3 | J |
| 10192 | ND3 | J |
| 10499 | ND4L | J |
| 10598 | ND4L | J |
| 10685 | ND4L | J |
| 11377 | ND4 | J |
| 12127 | ND4 | J |
| 12570 | ND5 | J |
| 12612 | ND5 | J |
| 13281 | ND5 | J |
| 13681 | ND5 | J |
| 13879 | ND5 | J |
| 13933 | ND5 | J |
| 14569 | ND6 | J |
| 15679 | CYT B | J |
| 15812 | CYT B | J |
| 16069 | D-LOOP | J |
| 16092 | D-LOOP | J |
| 16261 | D-LOOP | J |
| 114 | D-LOOP | K |
| 497 | D-LOOP | K |
| 593 | tRNA-F | K |
| 1189 | 12S rRNA | K |
| 2217 | 16S rRNA | K |
| 2483 | 16S rRNA | K |
| 3480 | ND1 | K |
| 4295 | tRNA-I | K |
| 4561 | ND2 | K |
| 5814 | tRNA-C | K |
| 6260 | COI | K |
| 9006 | ATPase6 | K |
| 9055 | ATPase6 | K |
| 9698 | COIII | K |
| 9716 | COIII | K |
| 9962 | COIII | K |
| 10289 | ND3 | K |
| 10550 | ND4L | K |
| 10978 | ND4 | K |
| 11299 | ND4 | K |
| 11470 | ND4 | K |
| 11485 | ND4 | K |
| 11840 | ND4 | K |
| 11869 | ND4 | K |
| 11923 | ND4 | K |
| 12954 | ND5 | K |
| 13135 | ND5 | K |
| 13740 | ND5 | K |
| 13967 | ND5 | K |
| 14002 | ND5 | K |
| 14037 | ND5 | K |
| 14040 | ND5 | K |
| 14167 | ND6 | K |
| 15884 | CYT B | K |
| 15946 | tRNA-T | K |
| 16224 | D-LOOP | K |
| 16234 | D-LOOP | K |
| 16463 | D-LOOP | K |
| 185 | D-LOOP | L1 |
| 186 | D-LOOP | L1 |
| 189 | D-LOOP | L1 |
| 236 | D-LOOP | L1 |
| 247 | D-LOOP | L1 |
| 297 | D-LOOP | L1 |
| 357 | D-LOOP | L1 |
| 710 | 12S rRNA | L1 |
| 825 | 12S rRNA | L1 |
| 1048 | 12S rRNA | L1 |
| 1738 | 16S rRNA | L1 |
| 2245 | 16S rRNA | L1 |
| 2395 | 16S rRNA | L1 |
| 2758 | 16S rRNA | L1 |
| 2768 | 16S rRNA | L1 |
| 2885 | 16S rRNA | L1 |
| 3308 | ND1 | L1 |
| 3516 | ND1 | L1 |
| 3666 | ND1 | L1 |
| 3693 | ND1 | L1 |
| 3777 | ND1 | L1 |
| 3796 | ND1 | L1 |
| 3843 | ND1 | L1 |
| 4312 | tRNA-I | L1 |
| 4586 | ND2 | L1 |
| 5036 | ND2 | L1 |
| 5393 | ND2 | L1 |
| 5442 | ND2 | L1 |
| 5603 | tRNA-A | L1 |
| 5655 | tRNA-A | L1 |
| 5913 | COI | L1 |
| 5951 | COI | L1 |
| 6071 | COI | L1 |
| 6150 | COI | L1 |
| 6185 | COI | L1 |
| 6253 | COI | L1 |
| 6548 | COI | L1 |
| 6827 | COI | L1 |
| 6989 | COI | L1 |
| 7055 | COI | L1 |
| 7076 | COI | L1 |
| 7146 | COI | L1 |
| 7337 | COI | L1 |
| 7389 | COI | L1 |
| 7867 | COII | L1 |
| 8248 | COII | L1 |
| 8428 | ATPase8 | L1 |
| 8655 | ATPase6 | L1 |
| 8784 | ATPase6 | L1 |
| 8877 | ATPase6 | L1 |
| 9042 | ATPase6 | L1 |
| 9072 | ATPase6 | L1 |
| 9347 | COIII | L1 |
| 9755 | COIII | L1 |
| 9818 | COIII | L1 |
| 10321 | ND3 | L1 |
| 10586 | ND4L | L1 |
| 10589 | ND4L | L1 |
| 10664 | ND4L | L1 |
| 10688 | ND4L | L1 |
| 10792 | ND4 | L1 |
| 10793 | ND4 | L1 |
| 10810 | ND4 | L1 |
| 11176 | ND4 | L1 |
| 11641 | ND4 | L1 |
| 11654 | ND4 | L1 |
| 11899 | ND4 | L1 |
| 12007 | ND4 | L1 |
| 12049 | ND4 | L1 |
| 12519 | ND5 | L1 |
| 12720 | ND5 | L1 |
| 12810 | ND5 | L1 |
| 13149 | ND5 | L1 |
| 13276 | ND5 | L1 |
| 13485 | ND5 | L1 |
| 13506 | ND5 | L1 |
| 13789 | ND5 | L1 |
| 13880 | ND5 | L1 |
| 13980 | ND5 | L1 |
| 14000 | ND5 | L1 |
| 14148 | ND5 | L1 |
| 14178 | ND6 | L1 |
| 14203 | ND6 | L1 |
| 14308 | ND6 | L1 |
| 14560 | ND6 | L1 |
| 14769 | CYT B | L1 |
| 14911 | CYT B | L1 |
| 15115 | CYT B | L1 |
| 15136 | CYT B | L1 |
| 16148 | D-LOOP | L1 |
| 16187 | D-LOOP | L1 |
| 16188 | D-LOOP | L1 |
| 16230 | D-LOOP | L1 |
| 16264 | D-LOOP | L1 |
| 16265 | D-LOOP | L1 |
| 16360 | D-LOOP | L1 |
| 16527 | D-LOOP | L1 |
| 143 | D-LOOP | L2 |
| 1442 | 12S rRNA | L2 |
| 1706 | 16S rRNA | L2 |
| 2332 | 16S rRNA | L2 |
| 2358 | 16S rRNA | L2 |
| 2416 | 16S rRNA | L2 |
| 2789 | 16S rRNA | L2 |
| 3495 | ND1 | L2 |
| 3918 | ND1 | L2 |
| 4158 | ND1 | L2 |
| 4185 | ND1 | L2 |
| 4370 | tRNA-Q | L2 |
| 4767 | ND2 | L2 |
| 5027 | ND2 | L2 |
| 5285 | ND2 | L2 |
| 5331 | ND2 | L2 |
| 5581 | tRNA-W | L2 |
| 5744 | NON-CODING | L2 |
| 6713 | COI | L2 |
| 7175 | COI | L2 |
| 7274 | COI | L2 |
| 7624 | COII | L2 |
| 7771 | COII | L2 |
| 8080 | COII | L2 |
| 8206 | COII | L2 |
| 8387 | ATPase8 | L2 |
| 8541 | ATPase8 | L2 |
| 8790 | ATPase6 | L2 |
| 8925 | ATPase6 | L2 |
| 9221 | COIII | L2 |
| 10115 | ND3 | L2 |
| 11944 | ND4 | L2 |
| 12236 | tRNA-S | L2 |
| 12630 | ND5 | L2 |
| 12693 | ND5 | L2 |
| 12948 | ND5 | L2 |
| 13803 | ND5 | L2 |
| 14059 | ND5 | L2 |
| 14544 | ND6 | L2 |
| 14566 | ND6 | L2 |
| 14599 | ND6 | L2 |
| 15110 | CYT B | L2 |
| 15217 | CYT B | L2 |
| 15229 | CYT B | L2 |
| 15236 | CYT B | L2 |
| 15244 | CYT B | L2 |
| 15391 | CYT B | L2 |
| 15629 | CYT B | L2 |
| 15945 | tRNA-T | L2 |
| 16114 | D-LOOP | L2 |
| 16213 | D-LOOP | L2 |
| 16309 | D-LOOP | L2 |
| 16390 | D-LOOP | L2 |
| 200 | D-LOOP | L3 |
| 2000 | 16S rRNA | L3 |
| 3438 | ND1 | L3 |
| 3450 | ND1 | L3 |
| 5773 | tRNA-C | L3 |
| 6524 | COI | L3 |
| 6587 | COI | L3 |
| 6680 | COI | L3 |
| 7424 | COI | L3 |
| 7618 | COII | L3 |
| 8616 | ATPase6 | L3 |
| 8618 | ATPase6 | L3 |
| 8650 | ATPase6 | L3 |
| 9554 | COIII | L3 |
| 10086 | ND3 | L3 |
| 10373 | ND3 | L3 |
| 10667 | ND4L | L3 |
| 10819 | ND4 | L3 |
| 11800 | ND4 | L3 |
| 13101 | ND5 | L3 |
| 13886 | ND5 | L3 |
| 13914 | ND5 | L3 |
| 14152 | ND6 | L3 |
| 14212 | ND6 | L3 |
| 14284 | ND6 | L3 |
| 15099 | CYT B | L3 |
| 15311 | CYT B | L3 |
| 15670 | CYT B | L3 |
| 15824 | CYT B | L3 |
| 15942 | tRNA-T | L3 |
| 15944 | tRNA-T | L3 |
| 16124 | D-LOOP | L3 |
| 16327 | D-LOOP | L3 |
| 930 | 12S rRNA | T |
| 2141 | 16S rRNA | T |
| 2850 | 16S rRNA | T |
| 4688 | ND2 | T |
| 4917 | ND2 | T |
| 5277 | ND2 | T |
| 6489 | COI | T |
| 7022 | COI | T |
| 8572 | ATPase6 | T |
| 8697 | ATPase6 | T |
| 9117 | ATPase6 | T |
| 9899 | COIII | T |
| 10463 | tRNA-R | T |
| 11242 | ND4 | T |
| 11812 | ND4 | T |
| 12633 | ND5 | T |
| 13368 | ND5 | T |
| 13758 | ND5 | T |
| 13965 | ND5 | T |
| 14233 | ND6 | T |
| 14687 | tRNA-E | T |
| 14905 | CYT B | T |
| 15028 | CYT B | T |
| 15274 | CYT B | T |
| 15607 | CYT B | T |
| 15928 | tRNA-T | T |
| 16163 | D-LOOP | T |
| 16182 | D-LOOP | T |
| 16186 | D-LOOP | T |
| 16294 | D-LOOP | T |
| 16296 | D-LOOP | T |
| 16324 | D-LOOP | T |
| 988 | 12S rRNA | U |
| 1700 | 16S rRNA | U |
| 1721 | 16S rRNA | U |
| 2294 | 16S rRNA | U |
| 3116 | 16S rRNA | U |
| 3197 | 16S rRNA | U |
| 3348 | ND1 | U |
| 3720 | ND1 | U |
| 3849 | ND1 | U |
| 4553 | ND2 | U |
| 4646 | ND2 | U |
| 4703 | ND2 | U |
| 4732 | ND2 | U |
| 4811 | ND2 | U |
| 5319 | ND2 | U |
| 5390 | ND2 | U |
| 5495 | ND2 | U |
| 5656 | NON-CODING | U |
| 5999 | COI | U |
| 6045 | COI | U |
| 6047 | COI | U |
| 6146 | COI | U |
| 6518 | COI | U |
| 6629 | COI | U |
| 6719 | COI | U |
| 7109 | COI | U |
| 7385 | COI | U |
| 7768 | COII | U |
| 7805 | COII | U |
| 8473 | ATPase8 | U |
| 9070 | ATPase6 | U |
| 9266 | COIII | U |
| 9477 | COIII | U |
| 9667 | COIII | U |
| 10506 | ND4L | U |
| 10876 | ND4 | U |
| 10907 | ND4 | U |
| 10927 | ND4 | U |
| 11197 | ND4 | U |
| 11332 | ND4 | U |
| 11732 | ND4 | U |
| 12346 | ND5 | U |
| 12557 | ND5 | U |
| 12618 | ND5 | U |
| 13020 | ND5 | U |
| 13617 | ND5 | U |
| 13637 | ND5 | U |
| 14139 | ND5 | U |
| 14179 | ND6 | U |
| 14182 | ND6 | U |
| 14620 | ND6 | U |
| 14793 | CYT B | U |
| 14866 | CYT B | U |
| 15191 | CYT B | U |
| 15218 | CYT B | U |
| 15454 | CYT B | U |
| 15693 | CYT B | U |
| 15907 | tRNA-T | U |
| 16051 | D-LOOP | U |
| 16256 | D-LOOP | U |
| 16270 | D-LOOP | U |
| 16399 | D-LOOP | U |
| 4580 | ND2 | V |
| 15904 | tRNA-T | V |
| 194 | D-LOOP | W |
| 1243 | 12S rRNA | W |
| 1406 | 12S rRNA | W |
| 3505 | ND1 | W |
| 6528 | COI | W |
| 8994 | ATPase6 | W |
| 10097 | ND3 | W |
| 11674 | ND4 | W |
| 11947 | ND4 | W |
| 12414 | ND5 | W |
| 13263 | ND5 | W |
| 15775 | CYT B | W |
| 15884 | CYT B | W |
| 16292 | D-LOOP | W |
| 225 | D-LOOP | X |
| 226 | D-LOOP | X |
| 6371 | COI | X |
| 8393 | ATPase8 | X |
| 8705 | ATPase6 | X |
| 14470 | ND6 | X |
| and | tRNA-T | X |
| 15927. | | |

6. A method for determining a mitochondrial haplogroup subgroup of a subject, comprising:
determining, in a biological sample comprising mitochondrial DNA from a subject, the presence of at least one mitochondrial single nucleotide polymorphism that is associated with a mitochondrial haplogroup subgroup.

7. The method of claim 6 wherein the mitochondrial haplogroup is selected from the group consisting of haplogroups K, U, J, T, W, I, H, V, X, L1, L2 and L3.

8. The method of claim 6 wherein at least one mitochondrial single nucleotide polymorphism that is associated with a mitochondrial haplogroup subgroup is a mitochondrial single nucleotide polymorphism located at a nucleotide that corresponds to a nucleotide position of SEQ ID NO:1 that is selected from the group consisting of position 3010, 16162, 16189, 16304, 1811, 3197, 9477, 14793, 16256, 13617, 16270, 7768, 14182, 3480, 9055, 9698, 10550, 11299, 14167, 14798, 16224, 16311, 1189, 10398, 497, 11470, 11914, 15924, 3010, 10398, 12612, 13798, 16069, 295, 489, 228, 462, 16193, 709, 1888, 4917, 8697, 10463, 13368, 14905, 15607, 15928, 16189, 16294, 5426, 6489, 11812, 15043, 16298, 12633, 16163 and 16186.

9. A method for determining a mitochondrial haplogroup subgroup of a subject, comprising:
determining in a biological sample comprising mitochondrial DNA from a subject of known mitochondrial haplogroup selected from the group consisting of haplogroups K, U, X, I, J, T, L1, L2 and L3, the presence or absence of a set comprising a plurality of single nucleotide polymorphisms wherein each polymorphism is located at a nucleotide that corresponds to a nucleotide position of SEQ ID NO:1, the set selected from the group consisting of:
a first haplogroup K subgroup comprising a polymorphism at positions 1147, 12308, 12372, 1811, 3480, 9055, 9698, 10550, 11299, 14167, 14798, 16224, 16311 and 709;
a second haplogroup K subgroup comprising a polymorphism at positions 1147, 12308, 12372, 1811, 3480, 9055, 9698, 10550, 11299, 14167, 14798, 16224, 16311, 1189 and 10398;
a third haplogroup K subgroup comprising a polymorphism at positions 1147, 12308, 12372, 1811, 3480, 9055, 9698, 10550, 11299, 14167, 14798, 16224, 16311, 1189, 10398 and 497;
a fourth haplogroup K subgroup comprising a polymorphism at positions 1147, 12308, 12372, 1811, 3480, 9055, 9698, 10550, 11299, 14167, 14798, 16224, 16311, 1189, 10398, 497 and 11914;
a fifth haplogroup K subgroup comprising a polymorphism at positions 1147, 12308, 12372, 1811, 3480, 9055, 9698, 10550, 11299, 14167, 14798, 16224, 16311, 1189, 10398, 497, 11914, 11470 and 15924;
a sixth haplogroup K subgroup comprising a polymorphism at positions 1147, 12308, 12372, 1811, 3480, 9055, 9698, 10550, 11299, 14167, 14798, 16224, 16311, 1189, 10398, 497; 11914, 11470, 15924, 12978 and 12954;
a seventh haplogroup K subgroup comprising a polymorphism at positions 1147, 12308, 12372, 1811, 3480, 9055, 9698, 10550, 11299, 14167, 14798, 16224, 16311, 1189, 10398, 497; 11914, 11470, 15924, 12978, 12954 and 114;
a first haplogroup U subgroup comprising a polymorphism at positions 11467, 12308, 12372 and 1811;
a second haplogroup U subgroup comprising a polymorphism at positions 11467, 12308, 12372, 3197, 9477, 13617, 16270;
a third haplogroup U subgroup comprising a polymorphism at positions 11467, 12308, 12372, 3197, 9477, 13617, 16270, 7768 and 14182;
a fourth haplogroup U subgroup comprising a polymorphism at positions 11467, 12308, 12372, 3197, 9477, 13617, 16270, 14793 and 16256;
a fifth haplogroup U subgroup comprising a polymorphism at positions 11467, 12308, 12372, 3197, 9477, 13617, 16270, 14793, 16256 and 15218;
a first haplogroup X subgroup comprising a polymorphism at positions 12705, 16223, 1719, 6221, 6371, 13966, 14470, 16278 and 225;
a second haplogroup X subgroup comprising a polymorphism at positions 12705, 16223, 1719, 6221, 6371, 13966, 14470, 16278, 225 and 226;
a first haplogroup I subgroup comprising a polymorphism at positions 12705, 16223, 1719, 10238, 10398, 12501, 13780 and 15043;
a second haplogroup I subgroup comprising a polymorphism at positions 12705, 16223, 1719, 10238, 10398, 12501, 13780, 15043, 250, 4529, 10034, 15924 and 16391;
a first haplogroup J subgroup comprising a polymorphism at positions 4216, 11251, 15452, 3010, 10398, 12612, 13708, 16069 and 16126;
a second haplogroup J subgroup comprising a polymorphism at positions 4216, 11251, 15452, 3010, 10398, 12612, 13708, 16069, 16126, 295 and 489;
a third haplogroup J subgroup comprising a polymorphism at positions 4216, 11251, 15452, 3010, 10398, 12612, 13708, 16069, 16126, 295, 489 and 15257;
a fifth haplogroup J subgroup comprising a polymorphism at positions 4216, 11251, 15452, 3010, 10398, 12612, 13708, 16069, 16126, 295, 489 and 462;
a sixth haplogroup J subgroup comprising a polymorphism at positions 4216, 11251, 15452, 3010, 10398, 12612, 13708, 16069, 16126, 295, 489, 462 and 228;
a first haplogroup T subgroup comprising a polymorphism at positions 709, 1888, 4917, 8697, 10463, 13368, 14905, 15607, 15928, 16126, 16294 and 12633;
a second haplogroup T subgroup comprising a polymorphism at positions 709, 1888, 4917, 8697, 10463, 13368, 14905, 15607, 15928, 16126, 16294, 12633, 16163 and 16186;
a third haplogroup T subgroup comprising a polymorphism at positions 709, 1888, 4917, 8697, 10463, 13368, 14905, 15607, 15928, 16126, 16294, 11812, 14233 and 16296;
a fourth haplogroup T subgroup comprising a polymorphism at positions 709, 1888, 4917, 8697, 10463, 13368, 14905, 15607, 15928, 16126, 16294, 11812, 14233, 16296, 930, 5147 and 16304;
a fifth haplogroup T subgroup comprising a polymorphism at positions 709, 1888, 4917, 8697, 10463, 13368, 14905, 15607, 15928, 16126, 16294, 11812, 14233, 16296, 5426, 6489 and 15043;
a first haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187 and 16311;
a second haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311 and 182;
a third haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 8027 and 16294;
a fourth haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 357, 709, 710, 1738, 2352, 2768, 3308, 3693, 5036, 5393, 5655, 6548, 6827, 6989, 7867, 8248, 12519, 13880, 14203, 15115, 16126 and 16264;
a fifth haplogroup L1 subgroup comprising a polymorphism at positions 8701, 9540, 10398, 10873, 12705, 16223, 769, 1018, 3594, 4104, 7256, 7521, 13650, 247, 825, 2758, 2885, 2666, 7055, 7146, 7389, 8468, 8655, 10688, 10810, 13105, 13506, 13789, 14178, 14560, 16187, 16311, 182, 357, 709, 710, 1738, 2352, 2768, 3308, 3693, 5036, 5393, 5655, 6548, 6827, 6989, 7867, 8248, 12519, 13880, 14203, 15115, 16126, 16264 and 14769;
a first haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278 and 16390;
a second haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390 and 182;
a third haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784 and 16294;
a fourth haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784, 16294 and 16309;
a fifth haplogroup L2 subgroup comprising a polymorphism at positions 8701, 9540, 10398, 10873, 12705, 16223, 769, 1018, 3594, 4104, 7256, 7521, 13650, 2416, 8206, 9221, 10115, 11944, 13590, 15301, 16278, 16390, 2789, 7175, 7274, 7771, 11914, 12693, 13803, 14566, 15784, 16294, 16309, 3918, 5285, 15244, 15629;
a first haplogroup L3 subgroup comprising a polymorphism at positions 8701, 9540, 10398, 10873, 12705, 16223, 15301;
a second haplogroup L3 subgroup comprising a polymorphism at positions 8701, 9540, 10398, 10873, 12705, 16223, 15301, 13105;
a third haplogroup L3 subgroup comprising a polymorphism at positions 8701, 9540, 10398, 10873, 12705, 16223, 15301, 13105, 3450, 5773, 6221, 9449, 10089, 10373, 13914, 15311, 15824, 15944, 16124, 16278 and 16362;
a fourth haplogroup L3 subgroup comprising a polymorphism at positions 8701, 9540, 10398, 10873, 12705, 16223, 15301, 489, 10400, 14783 and 15043;
a fifth haplogroup L3 subgroup comprising a polymorphism at positions 8701, 9540, 10398, 10873, 12705, 16223, 15301, 489, 10400, 14783, 15043, 2092, 3010, 4883, 5178, 6578, 14668 and 16325;
a sixth haplogroup L3 subgroup comprising a polymorphism at positions 8701, 9540, 10398, 10873, 12705, 16223, 15301, 489, 10400, 14783, 15043 and 16362;
a seventh haplogroup L3 subgroup comprising a polymorphism at positions 8701, 9540, 10398, 10873, 12705, 16223, 15301, 489, 10400, 14783, 15043, 4715, 7196, 8584 and 16298;
an eighth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325 and 16327;
a ninth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325, 16327, 289 and 290; and
a tenth haplogroup L3 subgroup comprising a polymorphism at positions 8701, 9540, 10398, 10873, 12705, 16223, 15301, 489, 10400, 14783, 15043, 4715, 7196, 8584, 16298, 249, 3552, 9545, 11914, 13263, 14318, 15487, 16325, 16327, 289, 290 and 15930.

10. A method for determining a genetic relationship between two subjects, comprising:
determining, in each of a first biological sample comprising mitochondrial DNA from a first subject and a second biological sample comprising mitochondrial DNA from a second subject, the presence or absence of at least one mitochondrial single nucleotide polymorphism,
wherein either (i) the presence of at least one mitochondrial single nucleotide polymorphism in both of said first and second biological samples, or (ii) the absence of at least one mitochondrial single nucleotide polymorphism from both of said first and second biological samples, indicates a genetic relationship between the subjects,
and therefrom determining the genetic relationship between the subjects.

11. The method of claim 10 wherein at least one mitochondrial single nucleotide polymorphism is associated with a mitochondrial haplogroup that is selected from the group consisting of haplogroups A, B, C, D, E, H, I, J, K, L1, L2, L3, T, U, V, W and X.

12. The method of claim 10 wherein at least one mitochondrial single nucleotide polymorphism that is associated with a mitochondrial haplogroup is a haplogroup-specific polymorphism that is present in members of only one haplogroup selected from the group consisting of A, B, C, D, E, H, I, J, K, L1, L2, L3, T, U, V, W and X, located at a nucleotide that corresponds to a nucleotide position of SEQ ID NO:1 that is selected from the group consisting of:

| Nucleotide | | HAPLO- |
|---|---|---|
| Position | GENE | GROUP |
| 64 | D-LOOP | A |
| 235 | D-LOOP | A |
| 663 | 12S rRNA | A |
| 1598 | 12S rRNA | A |
| 1736 | 16S rRNA | A |
| 3316 | ND1 | A |
| 4248 | ND1 | A |
| 4824 | ND2 | A |
| 4970 | ND2 | A |
| 6308 | COI | A |
| 7112 | COI | A |
| 7724 | COII | A |
| 8794 | ATPase6 | A |
| 11314 | ND4 | A |
| 12468 | ND5 | A |
| 12811 | ND5 | A |
| 13855 | ND5 | A |
| 14364 | ND6 | A |
| 16111 | D-LOOP | A |
| 16290 | D-LOOP | A |
| 16319 | D-LOOP | A |
| 827 | 12S rRNA | B |
| 3547 | ND1 | B |
| 4820 | ND2 | B |
| 4977 | ND2 | B |
| 6023 | COI | B |
| 6216 | COI | B |
| 6413 | COI | B |
| 6473 | COI | B |
| 6755 | COI | B |
| 7241 | COI | B |
| 9950 | COIII | B |
| 11177 | ND4 | B |
| 15535 | CYT B | B |
| 16217 | D-LOOP | B |
| 249 | D-LOOP | C |
| 289 | D-LOOP | C |
| 290 | D-LOOP | C |
| 3552 | ND1 | C |
| 4715 | ND2 | C |
| 7196 | COI | C |
| 7694 | COII | C |
| 8584 | ATPase6 | C |
| 9545 | COIII | C |
| 10454 | tRNA-R | C |
| 12642 | ND5 | C |
| 12978 | ND5 | C |
| 14318 | ND6 | C |
| 15487 | CYT B | C |
| 15930 | tRNA-T | C |
| 2092 | 16S rRNA | D |
| 4883 | ND2 | D |
| 5178 | ND2 | D |
| 8414 | ATPase8 | D |
| 11593 | ND4 | D |
| 14668 | ND6 | D |
| 3027 | 16S rRNA | E |
| 3705 | ND1 | E |
| 4491 | ND2 | E |
| 7598 | COII | E |
| 239 | D-LOOP | H |
| 456 | D-LOOP | H |
| 477 | D-LOOP | H |
| 951 | 12S rRNA | H |
| 961 | 12S rRNA | H |
| 3277 | tRNA-L | H |
| 3333 | ND1 | H |
| 3591 | ND1 | H |
| 3796 | ND1 | H |
| 3915 | ND1 | H |
| 3992 | ND1 | H |
| 4024 | ND1 | H |
| 4310 | tRNA-I | H |
| 4336 | tRNA-Q | H |
| 4531 | ND2 | H |
| 4727 | ND2 | H |
| 4745 | ND2 | H |
| 4772 | ND2 | H |
| 4793 | ND2 | H |
| 5004 | ND2 | H |
| 6365 | COI | H |
| 6776 | COI | H |
| 6869 | COI | H |
| 7013 | COI | H |
| 8269 | COII | H |
| 8448 | ATPase8 | H |
| 8602 | ATPase6 | H |
| 8803 | ATPase6 | H |
| 8839 | ATPase6 | H |
| 8843 | ATPase6 | H |
| 8898 | ATPase6 | H |
| 9123 | ATPase6 | H |
| 9150 | ATPase6 | H |
| 9380 | COIII | H |
| 9804 | COIII | H |
| 10044 | tRNA-G | H |
| 11353 | ND4 | H |
| 11560 | ND4 | H |
| 12579 | ND5 | H |
| 13404 | ND5 | H |
| 13680 | ND5 | H |
| 13759 | ND5 | H |
| 14125 | ND5 | H |
| 14350 | ND6 | H |
| 14365 | ND6 | H |
| 14470 | ND6 | H |
| 14582 | ND6 | H |
| 14872 | CYT B | H |
| 15466 | CYT B | H |
| 15789 | CYT B | H |
| 15808 | CYT B | H |
| 15833 | CYT B | H |
| 16162 | D-LOOP | H |
| 16293 | D-LOOP | H |
| 199 | D-LOOP | I |
| 250 | D-LOOP | I |
| 3447 | ND1 | I |
| 3990 | ND1 | I |
| 4529 | ND2 | I |
| 6734 | COI | I |
| 8616 | ATPase6 | I |
| 9947 | COIII | I |
| 10034 | tRNA-G | I |
| 10238 | ND3 | I |
| 11065 | ND4 | I |
| 12501 | ND5 | I |
| 13780 | ND5 | I |
| 15758 | CYT B | I |
| 16391 | D-LOOP | I |
| 228 | D-LOOP | J |
| 295 | D-LOOP | J |
| 462 | D-LOOP | J |
| 2158 | 16S rRNA | J |
| 2387 | 16S rRNA | J |
| 3394 | ND1 | J |
| 5198 | ND2 | J |
| 5633 | tRNA-A | J |
| 6464 | COI | J |
| 6554 | COI | J |
| 6671 | COI | J |
| 7476 | tRNA-S | J |
| 7711 | COII | J |
| 10084 | ND3 | J |
| 10172 | ND3 | J |
| 10192 | ND3 | J |
| 10499 | ND4L | J |
| 10598 | ND4L | J |
| 10685 | ND4L | J |
| 11377 | ND4 | J |
| 12127 | ND4 | J |
| 12570 | ND5 | J |
| 12612 | ND5 | J |
| 13281 | ND5 | J |
| 13681 | ND5 | J |
| 13879 | ND5 | J |
| 13933 | ND5 | J |
| 14569 | ND6 | J |
| 15679 | CYT B | J |
| 15812 | CYT B | J |
| 16069 | D-LOOP | J |
| 16092 | D-LOOP | J |
| 16261 | D-LOOP | J |
| 114 | D-LOOP | K |
| 497 | D-LOOP | K |
| 593 | tRNA-F | K |
| 1189 | 12S rRNA | K |
| 2217 | 16S rRNA | K |
| 2483 | 16S rRNA | K |
| 3480 | ND1 | K |
| 4295 | tRNA-I | K |
| 4561 | ND2 | K |
| 5814 | tRNA-C | K |
| 6260 | COI | K |
| 9006 | ATPase6 | K |
| 9055 | ATPase6 | K |
| 9698 | COIII | K |
| 9716 | COIII | K |
| 9962 | COIII | K |
| 10289 | ND3 | K |
| 10550 | ND4L | K |
| 10978 | ND4 | K |
| 11299 | ND4 | K |
| 11470 | ND4 | K |
| 11485 | ND4 | K |
| 11840 | ND4 | K |
| 11869 | ND4 | K |
| 11923 | ND4 | K |
| 12954 | ND5 | K |
| 13135 | ND5 | K |
| 13740 | ND5 | K |
| 13967 | ND5 | K |
| 14002 | ND5 | K |
| 14037 | ND5 | K |
| 14040 | ND5 | K |
| 14167 | ND6 | K |
| 15884 | CYT B | K |
| 15946 | tRNA-T | K |
| 16224 | D-LOOP | K |
| 16234 | D-LOOP | K |
| 16463 | D-LOOP | K |
| 185 | D-LOOP | L1 |
| 186 | D-LOOP | L1 |
| 189 | D-LOOP | L1 |
| 236 | D-LOOP | L1 |
| 247 | D-LOOP | L1 |
| 297 | D-LOOP | L1 |
| 357 | D-LOOP | L1 |
| 710 | 12S rRNA | L1 |
| 825 | 12S rRNA | L1 |
| 1048 | 12S rRNA | L1 |
| 1738 | 16S rRNA | L1 |
| 2245 | 16S rRNA | L1 |
| 2395 | 16S rRNA | L1 |
| 2758 | 16S rRNA | L1 |
| 2768 | 16S rRNA | L1 |
| 2885 | 16S rRNA | L1 |
| 3308 | ND1 | L1 |
| 3516 | ND1 | L1 |
| 3666 | ND1 | L1 |
| 3693 | ND1 | L1 |
| 3777 | ND1 | L1 |
| 3796 | ND1 | L1 |
| 3843 | ND1 | L1 |
| 4312 | tRNA-I | L1 |
| 4586 | ND2 | L1 |
| 5036 | ND2 | L1 |
| 5393 | ND2 | L1 |
| 5442 | ND2 | L1 |
| 5603 | tRNA-A | L1 |
| 5655 | tRNA-A | L1 |
| 5913 | COI | L1 |
| 5951 | COI | L1 |
| 6071 | COI | L1 |
| 6150 | COI | L1 |
| 6185 | COI | L1 |
| 6253 | COI | L1 |
| 6548 | COI | L1 |
| 6827 | COI | L1 |
| 6989 | COI | L1 |
| 7055 | COI | L1 |
| 7076 | COI | L1 |
| 7146 | COI | L1 |
| 7337 | COI | L1 |
| 7389 | COI | L1 |
| 7867 | COII | L1 |
| 8248 | COII | L1 |
| 8428 | ATPase8 | L1 |
| 8655 | ATPase6 | L1 |
| 8784 | ATPase6 | L1 |
| 8877 | ATPase6 | L1 |
| 9042 | ATPase6 | L1 |
| 9072 | ATPase6 | L1 |
| 9347 | COIII | L1 |
| 9755 | COIII | L1 |
| 9818 | COIII | L1 |
| 10321 | ND3 | L1 |
| 10586 | ND4L | L1 |
| 10589 | ND4L | L1 |
| 10664 | ND4L | L1 |
| 10688 | ND4L | L1 |
| 10792 | ND4 | L1 |
| 10793 | ND4 | L1 |
| 10810 | ND4 | L1 |
| 11176 | ND4 | L1 |
| 11641 | ND4 | L1 |
| 11654 | ND4 | L1 |
| 11899 | ND4 | L1 |
| 12007 | ND4 | L1 |
| 12049 | ND4 | L1 |
| 12519 | ND5 | L1 |
| 12720 | ND5 | L1 |
| 12810 | ND5 | L1 |
| 13149 | ND5 | L1 |
| 13276 | ND5 | L1 |
| 13485 | ND5 | L1 |
| 13506 | ND5 | L1 |
| 13789 | ND5 | L1 |
| 13880 | ND5 | L1 |
| 13980 | ND5 | L1 |
| 14000 | ND5 | L1 |
| 14148 | ND5 | L1 |
| 14178 | ND6 | L1 |
| 14203 | ND6 | L1 |
| 14308 | ND6 | L1 |
| 14560 | ND6 | L1 |
| 14769 | CYT B | L1 |
| 14911 | CYT B | L1 |
| 15115 | CYT B | L1 |
| 15136 | CYT B | L1 |
| 16148 | D-LOOP | L1 |
| 16187 | D-LOOP | L1 |
| 16188 | D-LOOP | L1 |
| 16230 | D-LOOP | L1 |
| 16264 | D-LOOP | L1 |
| 16265 | D-LOOP | L1 |
| 16360 | D-LOOP | L1 |
| 16527 | D-LOOP | L1 |
| 143 | D-LOOP | L2 |
| 1442 | 12S rRNA | L2 |
| 1706 | 16S rRNA | L2 |
| 2332 | 16S rRNA | L2 |
| 2358 | 16S rRNA | L2 |
| 2416 | 16S rRNA | L2 |
| 2789 | 16S rRNA | L2 |
| 3495 | ND1 | L2 |
| 3918 | ND1 | L2 |
| 4158 | ND1 | L2 |
| 4185 | ND1 | L2 |
| 4370 | tRNA-Q | L2 |
| 4767 | ND2 | L2 |
| 5027 | ND2 | L2 |
| 5285 | ND2 | L2 |
| 5331 | ND2 | L2 |
| 5581 | tRNA-W | L2 |
| 5744 | NON-CODING | L2 |
| 6713 | COI | L2 |
| 7175 | COI | L2 |
| 7274 | COI | L2 |
| 7624 | COII | L2 |
| 7771 | COII | L2 |
| 8080 | COII | L2 |
| 8206 | COII | L2 |
| 8387 | ATPase8 | L2 |
| 8541 | ATPase8 | L2 |
| 8790 | ATPase6 | L2 |
| 8925 | ATPase6 | L2 |
| 9221 | COIII | L2 |
| 10115 | ND3 | L2 |
| 11944 | ND4 | L2 |
| 12236 | tRNA-S | L2 |
| 12630 | ND5 | L2 |
| 12693 | ND5 | L2 |
| 12948 | ND5 | L2 |
| 13803 | ND5 | L2 |
| 14059 | ND5 | L2 |
| 14544 | ND6 | L2 |
| 14566 | ND6 | L2 |
| 14599 | ND6 | L2 |
| 15110 | CYT B | L2 |
| 15217 | CYT B | L2 |
| 15229 | CYT B | L2 |
| 15236 | CYT B | L2 |
| 15244 | CYT B | L2 |
| 15391 | CYT B | L2 |
| 15629 | CYT B | L2 |
| 15945 | tRNA-T | L2 |
| 16114 | D-LOOP | L2 |
| 16213 | D-LOOP | L2 |
| 16309 | D-LOOP | L2 |
| 16390 | D-LOOP | L2 |
| 200 | D-LOOP | L3 |
| 2000 | 16S rRNA | L3 |
| 3438 | ND1 | L3 |
| 3450 | ND1 | L3 |
| 5773 | tRNA-C | L3 |
| 6524 | COI | L3 |
| 6587 | COI | L3 |
| 6680 | COI | L3 |
| 7424 | COI | L3 |
| 7618 | COII | L3 |
| 8616 | ATPase6 | L3 |
| 8618 | ATPase6 | L3 |
| 8650 | ATPase6 | L3 |
| 9554 | COIII | L3 |
| 10086 | ND3 | L3 |
| 10373 | ND3 | L3 |
| 10667 | ND4L | L3 |
| 10819 | ND4 | L3 |
| 11800 | ND4 | L3 |
| 13101 | ND5 | L3 |
| 13886 | ND5 | L3 |
| 13914 | ND5 | L3 |
| 14152 | ND6 | L3 |
| 14212 | ND6 | L3 |
| 14284 | ND6 | L3 |
| 15099 | CYT B | L3 |
| 15311 | CYT B | L3 |
| 15670 | CYT B | L3 |
| 15824 | CYT B | L3 |
| 15942 | tRNA-T | L3 |
| 15944 | tRNA-T | L3 |
| 16124 | D-LOOP | L3 |
| 16327 | D-LOOP | L3 |
| 930 | 12S rRNA | T |
| 2141 | 16S rRNA | T |
| 2850 | 16S rRNA | T |
| 4688 | ND2 | T |
| 4917 | ND2 | T |
| 5277 | ND2 | T |
| 6489 | COI | T |
| 7022 | COI | T |
| 8572 | ATPase6 | T |
| 8697 | ATPase6 | T |
| 9117 | ATPase6 | T |
| 9899 | COIII | T |
| 10463 | tRNA-R | T |
| 11242 | ND4 | T |
| 11812 | ND4 | T |
| 12633 | ND5 | T |
| 13368 | ND5 | T |
| 13758 | ND5 | T |
| 13965 | ND5 | T |
| 14233 | ND6 | T |
| 14687 | tRNA-E | T |
| 14905 | CYT B | T |
| 15028 | CYT B | T |
| 15274 | CYT B | T |
| 15607 | CYT B | T |
| 15928 | tRNA-T | T |
| 16163 | D-LOOP | T |
| 16182 | D-LOOP | T |
| 16186 | D-LOOP | T |
| 16294 | D-LOOP | T |
| 16296 | D-LOOP | T |
| 16324 | D-LOOP | T |
| 988 | 12S rRNA | U |
| 1700 | 16S rRNA | U |
| 1721 | 16S rRNA | U |
| 2294 | 16S rRNA | U |
| 3116 | 16S rRNA | U |
| 3197 | 16S rRNA | U |
| 3348 | ND1 | U |
| 3720 | ND1 | U |
| 3849 | ND1 | U |
| 4553 | ND2 | U |
| 4646 | ND2 | U |
| 4703 | ND2 | U |
| 4732 | ND2 | U |
| 4811 | ND2 | U |
| 5319 | ND2 | U |
| 5390 | ND2 | U |
| 5495 | ND2 | U |
| 5656 | NON-CODING | U |
| 5999 | COI | U |
| 6045 | COI | U |
| 6047 | COI | U |
| 6146 | COI | U |
| 6518 | COI | U |
| 6629 | COI | U |
| 6719 | COI | U |
| 7109 | COI | U |
| 7385 | COI | U |
| 7768 | COII | U |
| 7805 | COII | U |
| 8473 | ATPase8 | U |
| 9070 | ATPase6 | U |
| 9266 | COIII | U |
| 9477 | COIII | U |
| 9667 | COIII | U |
| 10506 | ND4L | U |
| 10876 | ND4 | U |
| 10907 | ND4 | U |
| 10927 | ND4 | U |
| 11197 | ND4 | U |
| 11332 | ND4 | U |
| 11732 | ND4 | U |
| 12346 | ND5 | U |
| 12557 | ND5 | U |
| 12618 | ND5 | U |
| 13020 | ND5 | U |
| 13617 | ND5 | U |
| 13637 | ND5 | U |
| 14139 | ND5 | U |
| 14179 | ND6 | U |
| 14182 | ND6 | U |
| 14620 | ND6 | U |
| 14793 | CYT B | U |
| 14866 | CYT B | U |
| 15191 | CYT B | U |
| 15218 | CYT B | U |
| 15454 | CYT B | U |
| 15693 | CYT B | U |
| 15907 | tRNA-T | U |
| 16051 | D-LOOP | U |
| 16256 | D-LOOP | U |
| 16270 | D-LOOP | U |
| 16399 | D-LOOP | U |
| 4580 | ND2 | V |
| 15904 | tRNA-T | V |
| 194 | D-LOOP | W |
| 1243 | 12S rRNA | W |
| 1406 | 12S rRNA | W |
| 3505 | ND1 | W |
| 6528 | COI | W |
| 8994 | ATPase6 | W |
| 10097 | ND3 | W |
| 11674 | ND4 | W |
| 11947 | ND4 | W |
| 12414 | ND5 | W |
| 13263 | ND5 | W |
| 15775 | CYT B | W |
| 15884 | CYT B | W |
| 16292 | D-LOOP | W |
| 225 | D-LOOP | X |
| 226 | D-LOOP | X |
| 6371 | COI | X |
| 8393 | ATPase8 | X |
| 8705 | ATPase6 | X |
| 14470 | ND6 | X |
| and | tRNA-T | X |
| 15927. | | |

13. A method for determining a genetic relationship between (i) an unknown source or biological subject from which an unidentified sample is obtained, and (ii) a known source or biological subject from an identified sample is obtained, comprising:
determining presence or absence of at least one mitochondrial single nucleotide polymorphism, in each of a first biological sample derived from an unknown subject or biological source and a second biological sample derived from a known subject or biological source, wherein said first and second biological samples each comprise mitochondrial DNA,
wherein either (i) the presence of at least one mitochondrial single nucleotide polymorphism in both of said first and second biological samples, or (ii) the absence of at least one mitochondrial single nucleotide polymorphism from both of said first and second biological samples, indicates a genetic relationship between the subjects, and therefrom determining the genetic relationship between the biological samples.

14. A method of determining the presence of or the risk for having a disease associated with a mitochondrial DNA single nucleotide polymorphism, comprising:
(a) identifying at least one haplogroup-associated mitochondrial DNA single nucleotide polymorphism in a biological sample comprising mitochondrial DNA from a subject suspected of having or being at risk for having a disease associated with a mitochondrial DNA single nucleotide polymorphism; and
(b) identifying in said sample at least one disease associated mitochondrial DNA single nucleotide polymorphism that is not a haplogroup-associated mitochondrial DNA single nucleotide polymorphism, and therefrom determining the presence or risk of disease.

15. The method of claim 14 wherein the disease associated mitochondrial DNA single nucleotide polymorphism that is not a haplogroup-associated mitochondrial DNA single nucleotide polymorphism is a type 2 diabetes-associated polymorphism.

16. The method of claim 14 wherein the disease associated mitochondrial DNA single nucleotide polymorphism that is not a haplogroup-associated mitochondrial DNA single nucleotide polymorphism is an Alzheimer's disease-associated polymorphism.
